(19) United States
(12) Patent Application Publication
(10) Pub. No.: US 2009/0215895 A1
(43) Pub. Date: Aug. 27, 2009

(54) Therapeutic and carrier molecules

(75) Inventors: Antonio Ferrante, South Australia (AU); Deborah Ann Rathjen, South Australia (AU)

Correspondence Address: SCULLY, SCOTT, MURPHY & PRESSER, P.C., 400 GARDEN CITY PLAZA, SUITE 300, GARDEN CITY, NY, 11530, US

(73) Assignee: PEPLIN BIOLIPIDS PTY LTD, Newstead (AU)

(21) Appl. No.: 10/588,094

(22) PCT Filed: Jan. 28, 2005
(86) PCT No.: PCT/AU05/00098

§ 371 Date: May 7, 2009

Related U.S. Application Data
(60) Provisional application No. 60/540,604, filed on Jan. 30, 2004.

(51) Int. Cl. A61K 31/202; A61P 25/06; A61P 25/00; A61P 29/00; A61P 25/16; A61P 25/08; A61P 25/22; A61P 25/24; A61P 9/12; A61P 9/10; A61P 3/04; A61P 25/28; A61P 25/30; A61P 25/18; A61P 9/04

(52) U.S. Cl. 514/560

(57) ABSTRACT

The present invention relates generally to compounds comprising a hydrocarbon chain portion and more particular to compounds comprising chemical derivatizations of the hydrocarbon chain which are useful therapeutic and prophylactic molecules. The present invention further provides compounds where the hydrocarbon chain portion is a carrier molecule for functional groups, moieties or agents. The compounds of the present invention are particularly useful in the treatment and prophylaxis of a range of conditions including cancers, protein kinase c(PKC)- or NFkB-related- or -associated conditions, cardiovascular conditions, pain, inflammatory conditions, vascular or immunological conditions such as diabetes, neurological conditions and infection by a range of viruses or prokaryotic or eukaryotic organisms. The present invention further provides pharmaceutical compositions and methods of medical treatment.

### BACKGROUND OF THE INVENTION

1. Field of the Invention

The present invention relates generally to compounds comprising a hydrocarbon chain portion and more particular to compounds comprising chemical derivatizations of the hydrocarbon chain which are useful therapeutic and prophylactic molecules. The present invention further provides compounds where the hydrocarbon chain portion is a carrier molecule for functional groups, moieties or agents. The compounds of the present invention are particularly useful in the treatment and prophylaxis of a range of conditions including cancers, protein kinase c(PKC)- or NFκB-related- or -associated conditions, cardiovascular conditions, pain, inflammatory conditions, vascular or immunological conditions such as diabetes, neurological conditions and infection by a range of viruses or prokaryotic or eukaryotic organisms. The present invention further provides pharmaceutical compositions and methods of medical treatment.

2. Description of the Prior Art

Bibliographic details of references in the subject specification are also listed at the end of the specification.

Reference to any prior art in this specification is not, and should not be taken as, an acknowledgment or any form of suggestion that that prior art forms part of the common general knowledge in any country.

Fatty acids are one of the most extensively studied classes of compounds due to their important role in biological systems (Ferrante et al., In The Neutrophils: New outlook for the old cells [Ed Garbilovich] Imperial College Press 4:79-150, 1999; Sinclair and Gibson (Eds) Invited papers from the Third International Congress, American Oil Chemists'Society, Champaign, Ill., 1-482, 1992). Fatty acids consist of saturated, monosaturated and polyunsaturated fatty acids having a chain length from 4 to 30 carbon atoms. Polyunsaturated fatty acids (PUFAs) contain 16 to 30 carbon atoms with two or more methylene-interrupted cis-double bonds.

PUFA nomenclature includes recitation of the number of carbon atoms in the hydrocarbon chain, the number of double bonds and the position of the first double bond from the terminal methyl group (the ω-carbon atom). For example, the PUFA, arachidonic acid, contains 20 carbon atoms and four methylene-interrupted cis-double bonds commencing six carbons from the ω-carbon, viz: this PUFA is referred to as “arachidonic acid (20:6 n-6)”.

PUFAs can be divided into four families based on the fatty acids from which they are derived: linoleic acid (18:2 n-6), α-linolenic acid (18:3 n-3), oleic acid (18:1 n-9) and palmitoleic acid (16:1 n-7). The n-6 and n-3 PUFAs cannot be synthesized by mammals and are known as essential fatty acids (EFAs). They are acquired by mammalian bodies indirectly through desaturation or elongation of linoleic and α-linolenic acids, which must be supplied in the diet.

It is now well appreciated that ω-3 fatty acids confer some protection against a range of diseases. Synthetic fats have been synthesized which are useful in the treatment of a variety of conditions.

International Patent Publication Nos. WO 96/11908, WO 96/13507, WO 97/38688, WO 01/21172 and WO 01/21575 describe a range of PUFAs referred to as the MP Series, PT Series, Lx Series and MP-PT hybrid series. Some of these PUFAs, such as those of the MP Series, have reduced susceptibility to breakdown and, hence, are far less likely to cause the production of oxygen radicals which is the consequence of the metabolism of the natural ω-3 fatty acids. PT Series' PUFAs also have this property of resisting breakdown but in addition are more soluble. MP-PT hybrids are particularly useful anti-inflammatory agents.

As indicated above, naturally occurring ω-3 fatty acids have been found to be useful in treating a range of conditions including rheumatoid arthritis, multiple sclerosis, inflammatory bowel disease and systemic lupus. The PUFAs of the MP, PT, Lx and MP-PT hybrid series have also been proposed for the treatment of malaria, to stimulate or inhibit neutrophil activity, to treat T-cell diseases and in the treatment of cancer.

There is a need to determine the full range of activities of the PUFAs and to identify naturally occurring members or to generate synthetic derivatives which have therapeutic potential.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

In accordance with the present invention, it is proposed that the PUFAs are useful in the treatment inter alia of conditions associated with or involving protein kinase Cβ (PKCβ) and/or NFκB and in the treatment of pain, inflammation, vascular or immunological conditions such as diabetes, cardiovascular conditions, atherosclerosis, neurological conditions and infection by a range of viruses, prokaryotes or eukaryotes.

In particular, the present invention contemplates a method for the treatment or prophylaxis of a condition selected from the list consisting of an NFκB-related or -associated condition, a PKCβ-related or associated condition, vascular or immunological conditions such as diabetes, inflammation, neurological conditions, cardiovascular disease and pain in a subject, said method comprising administering to said subject an effective amount of a compound having the structure of Formula (I):

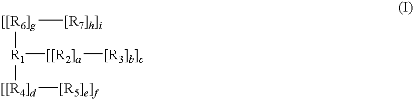

wherein
R₁ is a saturated or unsaturated hydrocarbon chain of from about 9 to about 26 carbon atoms and which optionally carries one or more of a oxa, thia, hydroxy, hydroperoxy, epoxy and peroxy substitution;
R₂, R₄, and R₆ may be the same or different and each is selected from O₂, NO, NO₂, S(O)ₓ, C(H)_{y}, H, COOH, P(X)_{δ}(Y), N(H)_{z}, C═O, OH,

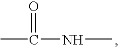

C₁₋₆ alkyl, C₁₋₆ alkoxy, amino, mono-acid di-C₁₋₆ alkylamino, C₁₋₆ alkylthio, S(O)ₓ—C₁₋₃ alkyl, C₁₋₆ alkoxycarbonyl, halo selected from fluoro, chloro, bromo and iodo, oxo, amidino and guanidino, C₂₋₁₂ alkenyl, C₂₋₁₂ alkynyl, aryl, heteroaryl and cyano, wherein x and z are 0, 1 or 2 and y is 0, 1, 2 or 3 and X is O, S or NR₈, Y is OR₉, SR₁₀ or NR₁₁R₁₂ and R₈, R₉, R₁₀, R₁₁ and R₁₂ are selected from H, alkyl, alkenyl, alkynyl, aryl and heteroaryl, δ is 0 or 1;
each of R₃, R₅ and R₇ is respectively [(CH₂)ⱼ(COOH)ₖ]ₗ, [(CH₂)ₘ(COOH)ₙ]ₒ and [(CH₂)ₚ(COOH)_{q}]ᵣ, wherein each of j, m and p is 0, 1, 2, 3, 4, 5 or 6, each of k, n and q is 0, 1 or 2, and each of l, o and r is 0 or 1,
each of c i and f is 0 or 1 or 2;
each of a, d and g is 0 or 1 or 2;
each of b, e and h is 0 or 1 or 2;
said administration being for a time and under conditions sufficient to prevent the condition or to ameliorate one or more symptoms of the condition.

The present invention extends to isolated naturally occurring PUFAs as well as synthetic or modified molecules. The subject molecules also include a range of hybrids in which the PUFA is conjugated to an L- or D-amino acid or a chemical analog of an amino acid.

The present invention further extends to compounds of general Formula (I) as defined above in isolated form or in a composition such as a pharmaceutical composition or formulation.

The present invention further provides for the use of a compound of general Formula (I) as defined above in the manufacture of a medicament for the treatment of a condition selected from the list consisting of a condition associated with or involving NFκB, PKCβ, pain, vascular or immunological conditions such as diabetes and cardiovascular disease, atherosclerosis, neurological conditions, inflammation and infection by a range of viruses, prokaryotes and eukaryotes.

The present invention also provides a compound of Formula (I):

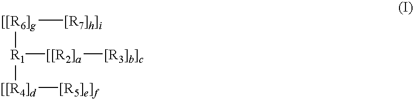

wherein
R₁ is a saturated or unsaturated hydrocarbon chain of from about 9 to about 26 carbon atoms and which is optionally carries one or more of a oxa, thia, hydroxy, hydroperoxy, epoxy and peroxy substitution;
R₂, R₄ and R₆ may be the same or different and each is selected from O₂, NO, NO₂, S(O)ₓ, C(H)_{y}, H, COOH, P(X)_{δ}(Y), N(H)_{z}, C═O, OH,

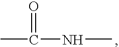

C₁₋₆ alkyl, C₁₋₆ alkoxy, amino, mono-acid di-C₁₋₆ alkylamino, C₁₋₆ alkylthio, S(O)ₓ—C₁₋₃ alkyl, C₁₋₆ alkoxycarbonyl, halo selected from fluoro, chloro, bromo and iodo, oxo, amidino and guanidino, C₂₋₁₂ alkenyl, C₂₋₁₂ alkynyl, aryl, heteroaryl and cyano, wherein x and z are 0, 1 or 2 and y is 0, 1, 2 or 3 and X is O, S or NR₈, Y is OR₉, SR₁₀ or NR₁₁R₁₂ and R₈, R₉, R₁₀, R₁₁ and R₁₂ are selected from H, alkyl, alkenyl, alkynyl, aryl and heteroaryl, δ is 0 or 1;
each of R₃, R₅ and R₇ is respectively [(CH₂)ⱼ(COOH)ₖ]ₗ, [(CH₂)ₘ(COOH)ₙ]ₒ and [(CH₂)ₚ(COOH)_{q}]ᵣ, wherein each of j, m and p is 0, 1, 2, 3, 4, 5 or 6, each of k, n and q is 0, 1 or 2, and each of l, o and r is 0 or 1,
each of c, i and f is 0 or 1 or 2; and
each of a, d and g is 0 or 1 or 2;
each of b, e and h is 0 or 1 or 2.

### BRIEF DESCRIPTION OF THE FIGURES

FIG. 1 is a diagrammatic representation showing the principle mechanism involving T-lymphocytes, leukocytes, macrophages and other cells of the immune system.

FIG. 2 is a graphical representation of the activation of neutrophil NADPH oxidase in the presence of 20 μM fatty acid as determined by lucigenin-dependent chemiluminescence.

FIG. 3 is a graphical representation showing the analgesic effects of PT2 in PQ writhing test.

FIG. 4 is a graphical representation showing the analgesic effects of PT2 in the formalin test.

FIG. 5 is a diagrammatic representation of a structure of MP3 (β-oxa-23:4n-6).

FIG. 6 is a diagrammatic representation showing the suppression of TNF-stimulated endothelial cell adhesion molecule expression by cells were pre-treated with MP3 (1 h) before being stimulated with TNF for the times indicated. Adhesion molecule expression was determined by ELISA.

FIG. 7 is a diagrammatic representation showing the suppression of LPS-stimulated leukocyte infiltration into the peritoneal cavity (a) and suppression of E-selectin expression by aortic endothelium (b) by MP3.

FIG. 8 is a diagrammatic representation showing the prevention of TNF-stimulated loss of IκBα in HUVEC by MP3 or 22:6n-3 cells were pre-treated with MP3 or 22:6n-3 (1 hr), stimulated with TNF (15 min) lysed and the lysate subjected to Western blot analysis using anti-IκBα antibody.

FIG. 9 is a diagrammatic representation showing the suppression of PKCβ1 translocation in glucose-stimulated mesangial cells (a) and in the glomeruli of a diabetic rat (b). Mesangial cells were pre-treated with MP5 or vehicle (ethanol) for 1 hr before being incubated with 25 mM glucose for 5 days. Male rats were rendered diabetic with streptozotocin and MP5 or vehicle (ethanol) was administered for 7 days after confirmation of diabetes. The cells and glomeruli were sonicated and particulate fraction-associated PKCβ1 was determined by Western blot analysis. High glucose and diabetes increased PKCβ1 in the particulate fraction. MP5 inhibited this effect.

FIG. 10 is a representation showing comparison of the ability of MP3 (β-oxa-23:4n-6) PMA (100 nmol/l) and 22:6n-3 to stimulate the neutrophil respiratory burst. Neutrophils were treated with DPC (Control), 23:4n-6, PMA or 22:6n-3 and then tested for chemiluminescence activity. The fatty acids were used at 20 μmol/l. The results are the mean±SEM of quadruplicates and is representative of two other experimental runs.

FIG. 11 is a representation showing effect of β-oxa; β-thia and natural PUFA on TNF-enhanced neutrophil adherence to HUVEC. HUVEC were pre-treated with the fatty acids (20 μmol/l) for 60 min at 37° C. before being stimulated with TNF (125 U/200 μl medium) for 4 hr at 37° C. The cells were then co-incubated with neutrophils (5×10⁵ cells/well) at 37° C. for 30 min and the degree of neutrophil adherence quantitated. The results are expressed as % of control and represent the mean±SEM of three separate experiments each performed in triplicate. * p<0.05, ***p<0.001, for significant differences between pre-treatment with fatty acid and control (one-way analysis of variance followed by the Dunnett test for multiple comparisons).

FIG. 12 is a representation showing effect of MP3 derivatives on TNF-enhanced neutrophil adherence to HUVEC. HUVEC were pre-treated with MP3 (20 μmol/l), β-oxa-23:4n-6 derivatives (20 μmol/l) or diluent (control) for 60 min and then challenged with TNF (125 U/200 μl medium) for a further 4 hr. The ability of HUVEC to adhere neutrophils was then assessed. The results are expressed as % of control and represent the mean±SEM of three separate experiments each performed in triplicate. ***p<0.001, for significant differences between pre-treatment with MP3 (β-oxa-23:4n-6) or derivative and control (one-way analysis of variance followed by the Dunnett test for multiple comparisons). Abbreviations used: β-oxa-23:4n-6ME, β-oxa-23:4n-6 methyl ester: β-oxa-23:0, saturated form of β-oxa-23:4n-6; β-oxa-23:4n-6OH, 18-monohydroxy-β-oxa-23:4n-6; β-oxa-23:4n-6OOH, 18-monohydroperoxy-β-oxa-23:4n-6.

FIG. 13 is a representation showing effect of MP3 (β-oxa-23:4n-6) and 20:4n-6 on time-related changes in TNF-α-induced E-selectin, ICAM-1 and VCAM-1 expression on HUVEC. HUVEC were pre-treated with 20 μmol/l β-oxa-23:4n-6 (closed triangles), 20 μmol/l 20:4n-6 (open squares), or DPC (control) for 60 min and then further incubated with TNF-α (125 U/200 μl medium) for up to 24 hr. The expression of E-selectin, ICAM-1 and VCAM-1 adhesion molecules was determined by ELISA. The results are expressed as % of control and represent the mean±SEM of three separate experiments each performed in triplicate. * p<0.05, **p<0.01, ***p<0.001, for significant differences between pre-treatment with fatty acid and corresponding control at a particular time point (one-way analysis of variance followed by the Dunnett test for multiple comparisons). Inset: The effect of β-oxa-23:4n-6 on TNF-α-induced expression of E-selectin mRNA in HUVEC. HUVEC were pre-incubated with β-oxa-23:4n-6 (20 μmol/l) or DPC (control) in 1 ml of medium at 37° C. for 60 min. After the addition of TNF-α, the cells were further incubated at 37° C. for 2 hr. E-selectin mRNA expression was then determined and the results expressed as relative %. Results are the mean±SEM of three separate experiments each performed in quadruplicate. *p<0.0001, for significant differences between pre-treatment with β-oxa-23:4n-6, and control (two-tailed Student's t-test for unpaired data).

FIG. 14 is a representation showing (A) effect of MP3 on in vivo inflammatory response measured as delayed type hypersensitivity (DTH) to sheep erythrocytes and LPS-induced influx of neutrophils and mononuclear cells in the peritoneal cavity in BALB/c mice. In the DTH experiments mice were injected with sheep erythrocytes subcutaneously, challenged with the antigen in the hind foot pad 6 days later and the amount of foot pad swelling measured 48 hr later. One hour prior to challenge mice were given 10 mg/kg body weight of β-oxa fatty acid in 7% w/v, DMSO as vehicle intraperitoneally. For the peritoneal cavity inflammation, mice were given intravenously 40 mg/kg MP3 intravenously and 6 hr later injected with LPS intraperitoneally. The cellular infiltrates were examined 24 and 72 hr later. The data, expressed as % of control, are presented as mean±SEM of 10 and 5 mice for DTH and peritoneal inflammation, respectively. Analysis of data by two-tailed student's t-test: **p<0.01, ***p<0.001. (B) Shows the effect of β-oxa-23:4n-6 on LPS-induced expression of E-selectin in aortic endothelium of BALB/C mice. Mice were treated intravenously with the fatty acid and 2 hr later injected intraperitoneally with LPS. After 5 hr the aortas were isolated, cut into small pieces and incubated with a monoclonal antibody to mouse E-selection (or isotype matched control) (Becton Dickinson, California) followed by an HRP-conjugated secondary antibody and then with the substrate ABTS (ELISA method). The data, expressed as % of control, are presented as mean±SEM of ten mice per group and is representative of two experimental runs. Analysis of the data by the two-tailed student's t-test: **p<0.01.

FIG. 15 is a representation showing the chemical structure of MP3 (β-oxa-23:4n-6) and of the monohydroxylated derivatives of β-oxa-23:4n-6 formed via the lipoxygenase pathway in HUVECs (15-monohydroperoxy-β-oxa-23:4n-6 was the predominant product).

FIG. 16 is a representation showing the effects of lipoxygenase/cyclooxygenase inhibitors and antioxidants on the modulation of E-selectin expression on HUVEC by β-oxa-23:4n-6. HUVEC were pre-treated with NDGA, baicalein, MK886, indomethacin, Vitamin E, or diluent (control) for 15 min. The cells were then further incubated with 20 μmol/l β-oxa-23:4n-6 or diluent (control) for 60 min followed by TNF-α (125 U/200 μl medium) for 4 hr and the expression of E-selectin adhesion molecule was determined. The results are expressed as % inhibition of the suppressive effect of β-oxa-23:4n-6 and represent the mean±SEM of three separate experiments each performed in quadruplicate. * p<0.01, for significant differences between pre-treatment with inhibitor and corresponding control (one-way analysis of variance followed by the Dunnett test for multiple comparisons).

FIG. 17 is a representation showing (A) the effect of MP3 (β-oxa 23:4n-6) and DHA on TNF-induced degradation of IκBα in HUVEC. Cells were pre-treated with the fatty acids (20 μmol/l) for 30 min and then stimulated with TNF (125 U/ml) for 10 min. After cell lysis the proteins were analyzed by Western blots using anti-IκBα antibodies. (B) The effects of β-oxa-23:4n-6 on TNF-induced activation of transcriptional factor, NFκB in HUVEC. Cells were pre-treated with β-oxa-23:4n-6 (20 μmol/l) for 30 min and then stimulated with TNF for 2 hr. After cell lysis, nuclear fractions were prepared, nuclear proteins separated by SDS PAGE (12% w/v gel), transferred to nitrocellulose and probed with an anti-NFκB p65 antibody (Santa Cruz). Densitometric analysis of data from three experiments showed that β-oxa 23:4n-6 reduced TNF-stimulated nuclear accumulation of NFκB by 66±2% (mean±SEM) (p<0.001, two-tailed student's t-test). (C) The effect of β-oxa 23:4n-6 on TNF-stimulated activation of IKK. Cells were pre-treated with β-oxa 23:4n-6 (20 μmol/l) for 30 min and then stimulated with TNF for 5 min. After cell lysis IKK was immunoprecipitated with anti-IKKα antibody and kinase activity determined.

### DETAILED DESCRIPTION OF THE PREFERRED EMBODIMENTS

The present invention provides compounds of general Formula (I):

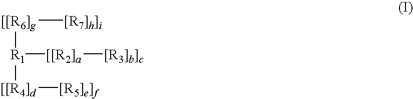

wherein
R₁ is a saturated or unsaturated hydrocarbon chain of from about 9 to about 26 carbon atoms and which is optionally carries one or more of a oxa, thia, hydroxy, hydroperoxy, epoxy and peroxy substitution;
R₂, R₄ and R₆ may be the same or different and each is selected from O₂, NO, NO₂, S(O)ₓ, C(H)_{y}, H, COOH, P(X)_{δ}(Y), N(H)_{z}, C═O, OH,

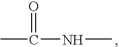

C₁₋₆ alkyl, C₁₋₆ alkoxy, amino, mono-acid di-C₁₋₆ alkylamino, C₁₋₆ alkylthio, S(O)ₓC₁₋₃ alkyl, C₁₋₆ alkoxycarbonyl, halo selected from fluoro, chloro, bromo and iodo, oxo, amidino and guanidino, C₂₋₁₂ alkenyl, C₂₋₁₂ alkynyl, aryl, heteroaryl and cyano, wherein x and z are 0, 1 or 2 and y is 0, 1, 2 or 3 and X is O, S or NR₈, Y is OR₉, SR₁₀ or NR₁₁R₁₂ and R₈, R₉, R₁₀, R₁₁ and R₁₂ are selected from H, alkyl, alkenyl, alkynyl, aryl and heteroaryl, δ is 0 or 1;
each of R₃, R₅ and R₇ is respectively [(CH₂)ⱼ(COOH)ₖ]ₗ, [(CH₂)ₘ(COOH)ₙ]ₒ and [(CH₂)ₚ(COOH)_{q}]ᵣ, wherein each of j, m and p is 0, 1, 2, 3, 4, 5 or 6, each of k, n and q is 0, 1 or 2, and each of l, o and r is 0 or 1,
each of c, i and f is 0 or 1 or 2; and
each of a, d and g is 0 or 1 or 2;
each of b, e and h is 0 or 1 or 2.

More particularly, the present invention contemplates a method for the treatment or prophylaxis of a condition selected from the list consisting of an NFκB-related or -associated condition, a PKCβ related or associated condition, vascular or immunological conditions such as diabetes, inflammation, neurological conditions, cardiovascular disease and pain in a subject, said method comprising administering to said subject an effective amount of a compound having the structure of Formula (I):

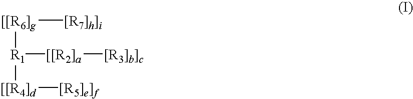

wherein
R₁ is a saturated or unsaturated hydrocarbon chain of from about 9 to about 26 carbon atoms and which is optionally carries one or more of a oxa, thia, hydroxy, hydroperoxy, epoxy and peroxy substitution;
R₂, R₄ and R₆ may be the same or different and each is selected from O₂, NO, NO₂, S(O)ₓ, C(H)_{y}, H, COOH, P(X)_{δ}(Y), N(H)_{z}, C═O, OH,

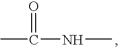

C₁₋₆ alkyl, C₁₋₆ alkoxy, amino, mono-acid di-C₁₋₆ alkylamino, C₁₋₆ alkylthio, S(O)ₓ—C₁₋₃ alkyl, C₁₋₆ alkoxycarbonyl, halo selected from fluoro, chloro, bromo and iodo, oxo, amidino and guanidino, C₂₋₁₂ alkenyl, C₂₋₁₂ alkynyl, aryl, heteroaryl and cyano, wherein x and z are 0, 1 or 2 and y is 0, 1, 2 or 3 and X is O, S or NR₈, Y is OR₉, SR₁₀ or NR₁₁R₁₂ and R₈, R₉, R₁₀, R₁₁ and R₁₂ are selected from H, alkyl, alkenyl, alkynyl, aryl and heteroaryl, δ is 0 or 1;
each of R₃, R₅ and R₇ is respectively [(CH₂)ⱼ(COOH)ₖ]ₗ, [(CH₂)ₘ(COOH)ₙ]ₒ and [(CH₂)ₚ(COOH)_{q}]ᵣ, wherein each of j, m and p is 0, 1, 2, 3, 4, 5 or 6, each of k, n and q is 0, 1 or 2, and each of l, o and r is 0 or 1,
each of c i and f is 0 or 1 or 2;
each of a, d and g is 0 or 1 or 2;
each of b, e and h is 0 or 1 or 2;
said administration being for a time and under conditions sufficient to prevent the condition or to ameliorate one or more symptoms of the condition.

The compound of Formula (I) may comprise, when i, c and f are 0, a straight hydrocarbon chain such as that shown in Formula (II):

[C(H)_{a′}]_{a″} (II)
which represents a hydrocarbon chain of a″ carbons in length from about 9 to about 26 carbon atoms, which hydrocarbon chain is saturated or unsaturated and which carries one or more of a oxa, thia, hydroxy, hydroperoxy, epoxy and/or peroxy substitution; a′ may be 0, 1, 2 or 3.

The compound of Formula I may also comprise two of l, c or f being 0 and one of the remaining i, c or f being 1. For example, where i and f are each 0, the resulting compound has the structure of Formula (III):

R₁-[R₂]ₐ-[R₃]_{b} (III)

wherein R₁, R₂, R₃, a and b are as defined above.

When the compound of Formula (III) comprises each of, a, o and b being 1, the resulting compound has the structure of Formula (IV):

R₁-R₃ (IV)

wherein R₁ and R₃ are as defined above.

Given that R₃ is [(CH₂)ⱼ(COOH)ₖ]ₗ, Formula (IV) can be represented as a compound of Formula (V):

R₁—[(CH₂)ⱼ(COOH)ₖ]ₗ (V)

wherein R₁, j, k and l are as represented above.

In a preferred embodiment, l is a saturated or unsaturated fatty acid. In another preferred embodiment, the saturated or unsaturated fatty acid carries one or more of a β-oxa, α-oxa, γ-oxa, β-thia, α-thia, γ-thia, hydroxy, hydroperoxy, epoxy, peroxy, peracetyl or other protected hydroperoxy substitution. Substitutions may be at the level of a carbon atom or hydrogen atom.

Examples of compounds of Formula (V) include:

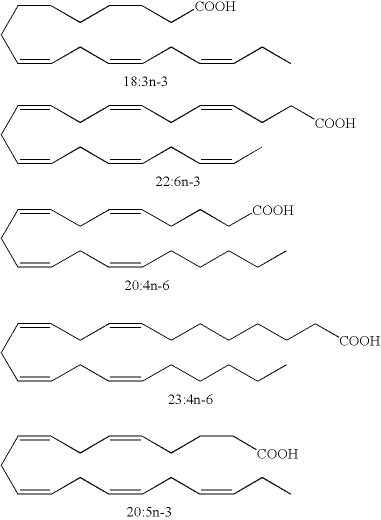

Examples of compounds where R₁ comprises a substitution include:

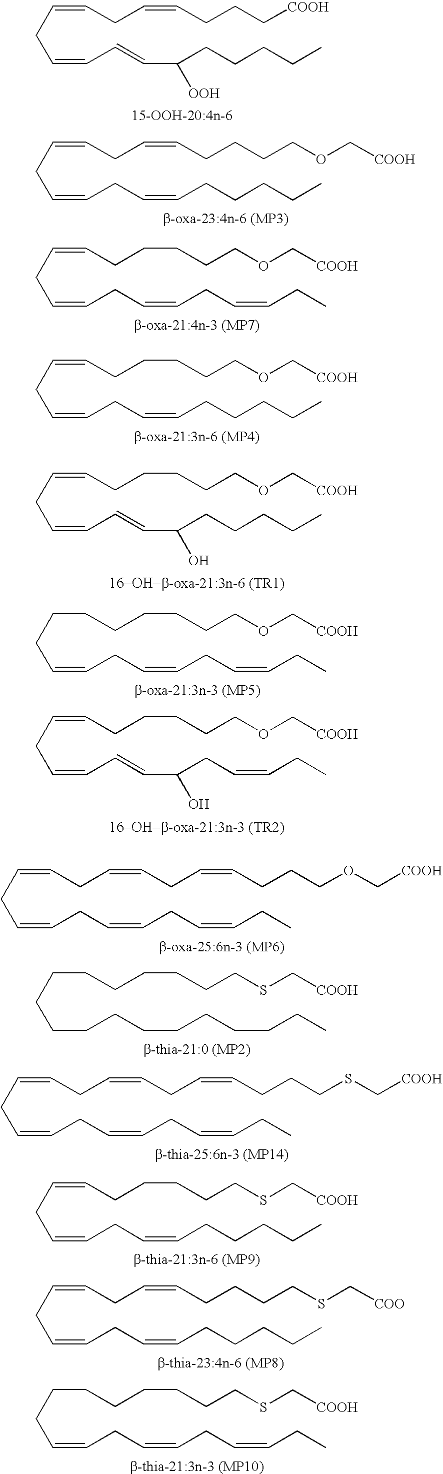

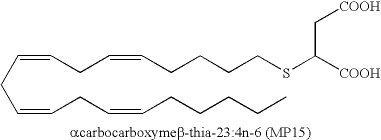

When each of [[R₆]_{g}-[R₇]ₕ]ᵢ, [[R₂]ₐ-[R₃]_{b}]_{c} and/or [[R₄]_{d}-[R₅]ₑ]_{f} are presented in multiple forms, then the multiple forms may be represented linearly. For example, if i and f are each 0, a is 3, b is 1 and c is 1, then the compound may be represented as in Formula (VI):

R₁-R₂-R₂-R₂-R₃ (VI)

If, on the other hand, c is 2, then the compound is represented as Formula (VII):

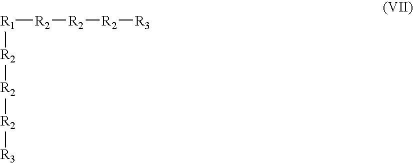

In one non-limiting example, in the case when the compound is a carboxymethyl derivative, then the values in Formula (I) are as follows:
i is 0, each of c and f is 1, each of a and d is 0 and each of R₃ and R₅ is [(CH₂)ⱼ(COOH)ₖ]ₗ and [(CH₂)ₘ(COOH)ₙ]ₒ, respectively where, in one example,
each of j and m is 0,
each of l and o is 1; and
each of k and n is 1,
resulting in a compound of Formula (VIII):

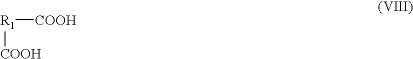

More commonly, however, j may be 1, and m may be 2 resulting a compound of Formula (IX):

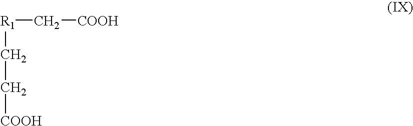

Reference to “from about 9 to about 26 carbon atoms” herein includes 9, 10, 11, 12, 13, 14, 15, 16, 17, 18, 19, 20, 21, 22, 23, 24, 25 and 26 carbon atoms.

The compound of Formula (I) may have each of i, c and f as 0 (zero), two of i, c and f as 0 (zero) or one of i, c and f as 0 (zero); or each of i, c and f as 1; two of i, c and f as 1 or one of i, c and f as 1; or each of i, c and f as two, two of i, c and f as two, or one of i, c and f as two.

The compound of Formula (I) may have each of g, a and d as 0 (zero), two of g, a and d as 0 (zero) or one of g, a and d as 0 (zero); or each of g, a and d as 1; two of g, a and d as 1 or one of g, a and d as 1; or each of g, a and d as two, two of g, a and d as two, or one of g, a and d as two.

The compound of Formula (I) may have each of h, b and e as 0 (zero), two of h, b and e as 0 (zero) or one of h, b and e as 0 (zero); or each of h, b and e as 1; two of h, b and e as 1 or one of h, b and e as 1; or each of h, b and e as two, two of h, b and e as two, or one of h, b and e as two.

These aspects of the present invention cover naturally occurring PUFAs as well as synthetic, modified or derivatized PUFAs. Furthermore, modified PUFAs encompassed by Formulae (I) through (VIII) include naturally occurring or synthetic, derivatized or modified PUFAs conjugated to an L- or D-amino acid or amino acid analog or a sequence of amino acids such as in peptide, polypeptide or a protein. The latter aspect includes proteins in the form of cytokines, growth factors, proteases, enzymes, apoptotic proteins and pro-survival proteins.

Examples of L-amino acids include alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and valine.

Examples of chemical analogs of amino acids include, but are not limited, to α-aminobutyric acid, α-amino-α-methylbutyrate, aminocyclopropane-, carboxylate, aminoisobutyric acid, aminonorbornyl-, carboxylate, cyclohexylalanine, cyclopentylalanine, D-alanine, D-arginine, D-aspartic acid, methylmethionine, D-cysteine, N-methylnorleucine, D-glutamine, D-glutamic acid, methylornithine, D-histidine, N-methylphenylalanine, D-isoleucine, D-leucine, D-lysine, D-methionine, D-ornithine, D-phenylalanine, D-proline, D-serine, D-threonine, D-tryptophan, D-tyrosine, D-valine, D-α-methylalanine, D-α-methylarginine, D-α-methylasparagine, D-α-methylaspartate, D-α-methylcysteine, D-α-methylglutamine, D-α-methylhistidine, D-α-methylisoleucine, D-α-methylleucine, D-α-methyllysine, D-α-methylmethionine, D-α-methylornithine, D-α-methylphenylalanine, D-α-methylproline, D-α-methylserine, D-α-methylthreonine, D-α-methyltryptophan, D-α-methyltyrosine, D-α-methylvaline, D-N-methylalanine, D-N-methylarginine, D-N-methylasparagine, D-N-methylaspartate, D-N-methylcysteine, D-N-methylglutamine, D-N-methylglutamate, D-N-methylhistidine, D-N-methylisoleucine, D-N-methylleucine, D-N-methyllysine, N-methylcyclohexylalanine, D-N-methylornithine, N-methylglycine, N-methylaminoisobutyrate, N-(1-methylpropyl)glycine, N-(2-methylpropyl)glycine, D-N-methyltryptophan, D-N-methyltyrosine, D-N-methylvaline, γ-aminobutyric acid, L-t-butylglycine, L-ethylglycine, L-homophenylalanine, L-α-methylarginine, L-α-methylaspartate, L-α-methylcysteine, L-α-methylglutamine, L-α-methylhistidine, L-α-methylisoleucine, L-α-methylleucine, L-α-methylmethionine, L-α-methylnorvaline, L-α-methylphenylalanine, L-α-methylserine, L-α-methyltryptophan, L-α-methylvaline, N—(N-(2,2-diphenylethyl)carbamylmethyl)glycine and 1-carboxy-1-(2,2-diphenyl-ethylamino)cyclopropane.

Examples of cytokines include but are not limited to BDNF, CNTF, EGF, EPO, FGF1, FGF2, FGF3, FGF4, FGF5, FGF6, FGF7, FGF8, FGF9, FGF10, FGF11, FGF12, FGF12, FGF13, FGF14, FGF15, FGF16, FGF17, FGF18, FGF19, FGF20, FGF21, FGF22, FGF23, G-CSF, GM-CSF, IFNα, IFNβ, IFNγ, IL1, IL2, IL3, IL4, IL5, IL6, IL7, IL8, IL9, IL10, IL11, IL12, IL13, IL14, IL15, LIF, MCP1, MCP2, MCP3, MCP4, MCP5, M-CSF, MIP1, MIP2, NGF, NT 3, NT4, NT5, NT6, NT7, OSM, PBP, PBSF, PDGF, PF4, RANTES, SCF, TGFα, TGFβ, TNFα, TNFβ, TPO, VEGF, GH, insulin and the like.

Examples of apoptotic proteins include but are not limited to A1, A9, A20, A46R, A52R, A53, A238L, Aac11, AATF, AATYK, ABIN1, ABIN-1, ABIN2, acidic sphigomyelinase, Acinus, Act1, Act2, activin, AD3LP, AD5, ADAR, adrenomedullin, aggrecan, AMAM17, 33, AI1, AIF, AILIM, AIM2, AIR, AITR, Akt, ALCAM, ALG2, ALG3, ALG4, ALP, Alix, Armadillo, AMAC1, AMH, AMID, Amida, angiotensinogen, Ankyrin, ANT1, AO7, AP1, Apaf-1, APC, APC2, APCL, APE1820, APJ, APO-1, APO-2, APO-3, Apopain, APP1, APP2, Apr, APRIL, ARA54, ARC, ARF, arkadia, ARIH1, 2, ASC, Ash2, Ask1, Ask2, ASPP1, ASPP2, AT2R1, AT2R2, ATAR, ATF1, ATF2, ATF3, ATF4, ATM, atona, ATR1, AUF1, Aven, AVP, AvrA, AvrBsT, Axam, Axin, Axin 2, Axi, b-catenin, b-TrCP, B28R, B7-1, B7-2, B7h2, B7RP1, Bach2, Bad, BAFF, BAG-1, -2, -3, -4, -5, Bak, BALF1, Bam32, BAP-1, BAP31, BAP29, BAR, BARD1, BAT3, Bax, BBc3, BCA1, BCAN, Bcl-2, BCL2, Bcl-3, Bcl-10, BCL10, Bcl-G, Bcl-Rambo, Bcl-w, Bcl-x, beclin, BEHAB, BERP, Bfl-1, BFL1, BG1, BG2, BG4, BG5, BHP1, BHRF1, BI-1, Bid, Bif-1, Bik, Bis, Bim, Bimp-1, Bimp1, Bimp2, Bimp3, BIR1, BIRP, BL-CAM, BLC, Blk, BLNK, BLR1, BLyS, BMI-1, BmP109, BNIP3, BNIP3a, BNIP3L, Bok, bone sialoprotein, bonus, Boo, BPI, BRAL1, BRAG-1, BRAP, Bravo, BRCA1, BRN3a, BRN3b, BRN3c, brevican, BPR, BSAC, BUFFY, C1q, C1r, C1s, C2, C3, C4a, C4b, C5, C6, C7, C8a, C8b, C8g, C9, C1qBP, C3aR, C4BPa,b, C5R1, CR2, CIITA, C5L, c-E10, c-FLIP, c-Fms, c-Fos, c-IAP1, cIAP1, c-IAP-1, c-IAP2, cIAP2, c-IAP-2, c-Jun, c-Myc, c-Rel, cactus, CAD, cadherin, E, N, P, VE, calcineurin, CARD4, CARD7, CARD9, CARD10, CARD11, CARD12, CARD14, CARDIAK, Carmal, CARMA-1, CARMA2, CARMA3, CARMA, CARMEN, CAP1, CAR1, CART1, CAS, CAS-L, caspase-1, -2, -3, 4, -5, -6, -7, -8, -9, -11, -12, -13, -14, Casper-1, -2, -3, -4, -5, -6, -7, -8, -9, -10, -11, -12, -13, -14, -15, -16, -17, -18, -19, -20, -21, -22, -23, -24, -25, -26, -27, -28, CASH, CBL, CBL-B, CBL-C, CC-CKR-6, CCF, CCL, CCPI, CCRs, CD2, CD3, CD4, CD5, CD6, CD7, CD8, CD9, CD11, CD14, CD18, CD19, CD20, CD21 (CR2), CD22, CD23, CD25, CD27, CD27L, CD28, CD28LG1, CD28LG2, CD29, CD30, CD31, CD32, CD33, CD34, CD35, CD36, CD40, CD40L, CD41, CD43, CD44, CD45, CD46, CD47, CD48, CD49, CD50, CD53, CD54, CD55, CD56, CD58, CD59, CD61, CD62E, L, H, CD66, CD63, CD64, CD66a-e, CD67, CD70, CD72, CD74, CD79a, b, CD80, CD84, CD85a-m, CD86, CD88, CD89, CD90, CD92, CD94, CD95, CD96, CD97, CD99, CD100, CD101, CD102, CD104, CD105, CD106, CD108, CD112, CD115, CD116, CD117, CD119, CD120a-b, CD121a-b, CD122, CD123, CD124, CD125, CD126, CD127, CD128a-b, CD130, CD131, CD132, CD134, CD135, CD136, CD137, CD140a, CD140b, CD143, CD144, CD146, CD147, CD148, CD150, CD151, CD152, CD153, CD154, CD155, CD158a-z, CD159, CD160, CD161, CD162, CD166, CD178, CD180, CD183, CD184, CD195, CD197, CD207, CD229, CD244, CDC2, CDC25, CDC42, CDK1, CDK2, CDK5, CDM, CEA, CEAL, CEACAM1, 6, C/EBP, CED1, CED2, CED3, CED4, CED5, CED6, CED7, CED8, CED9, Ced-9, CED10, CED11, CED12, CED, CEP-1, CES1, CES2, CES3, CETP, CeTRAF, Cezanne, CGR19, CGRP, Che1, Che-1, CHFR, chemokines, CHOP, CHUK, cIAP1, cIAP2, c-IAP1, c-IAP2, c-IAP-1, c-IAP-2, CIDE-A, CIDE-B, CIKS, CIN85, CIP-1, CIPER, CISK, Ckb-8, CKR1, 2, 3, 4, 5, CKRL1, Clan, CLAP, CLARP, CMD1, CMH1, CMKBR1, 2, 3, 4, 5, 6, CMPD1, conductin, Cop9 subunit 3, COP11, COPS3, COPS5, COT, COX-1, COX-2, CPAN, CPP32, CPZ, CRADD, CRAF1, CR8, CREB, CREM, Crk-II, crinkled, crmA, crmB, CSBP1, CSMF, CSN3, Csp-1, Csp-2, Csp-3, CSPG2, 3, Csx, CTACK, CTAP3, CTGF, CTLA4, cytochrome c, cytosolic PL A2, CXCLs, CXC-R₃, DAAM1, Dad1, DAD-1, Damm, DAP1, DAP3, DAP5, DAP12, DAP kinase 1, DAPP1, DAXX, Dborg1, dCAD, DCCK1, DCP1, Dcp-1, Dcp-2, DcR-1, DcR-2, DcR-3, DD2, Decay, DED, DEDAF, DEDD, DEDD2, dedprol, defensin, DEFT, dFADD, DFF, DFF35, DFF40, DFF45, DG17, Diablo, DIAP1, DIAP2, Dickkopf, DIF, DIF2, DIHA, DIK, Drosophila IKK, PKCδ-interacting protein kinase, DIO1, DIP, disheveled, diubiquitin, DKK-1, DKK-2, DKK-3, DKK-4, DLAK, DLK, DMDL, DNase II, Diva, DONG1, Dorsal, DP1, DP2, DP5, Drob1, DRP-1, DocA, dock188, Dok1, Doom, dorfin, DR3,4,5,6, DRAK 1-2, DREAM, DREP-1, DREP-2, DREP-3, DREP-4, DrICE, DRONC, DRP1, DTR, DTS, DUSP, E1.1, E1B 19K, E10, E2Fs, E4BP4, E4ORF4, E8, E4, E48, E3RS, eae7, Ear7, EBAF, EBI1, EBP1, EBI6, ECSIT, EDA, EDAR, Edradd, EFP, EGL1, Egr1-2-3, EHF, eIF-2aK, Eiger, ELAM, ELF2, ELK1-4, EMR1, ENA78, Endofin, Endoglin, Endophilin B1, endothelin, ENG, eNOS, eotaxin 1,2, ERN1, ERICE, ES18, Ets-1-2, ER81, ErbAa, ERG, ERM, ESE2, Eskine, ETV1, 2,3,4,5,6, exodus-1, exodus-2, exodus-3, FADD, Fas associated via death domain, FAF1, FAIM, FAN, FANCC, Fas, FAST, FAT10, fb1, FCAR, FELL, FEM-1, FEM-2, FHR1-2, FHR-3, FHR-4, FHR-5, FKBPs, FIGF, FIL1d, e, eta, zeta, FIP2, FIP3, FKSG2, FIST, FKHL12, FKHR, FKHRL1, FLAME-1, FLAME-3, FLAME3, FLASH, FLDED-1, FLI-1, FLI1, FLICE, FLICE2, FLICE-2, FLIP, FLT3L, Fliz1, Fln29, Fms, Fnk, fortilin, Fos, FOXO1A, FOXO3A, FOXE3, FPV039, Fra1, Fra2, Fractalkine, FRAP, FREAC8, Frizzled, Fzd, Fz, FRING, FRP1-2-3, FRP1(ATR), frpHE, FRZB-PEN, Fsp27, FUS, FUS6, Fusin, FXY, FY, G-coupled receptors, G10P1, G25K, G4R, G6C, G6E, GADD34, GADD45, GADD153, GATA1,2,3,4,5,6, GBP2, GCP2, GDFs, gelsolin, Gfi-1, Gfi1, GFRP1, GILZ, gingipain, GITR, GL50, glycodelin A, GM2A, gp34, GPR5, GPR9, GPR-9-6, Granzyme B, Grim, GRMP, Groa, Grob, GRS, GSKβ, H2TF1, H731-like, Hakai, HB-EGF, Hck, HF1, HFB30, HFL3, HHARI, hIAP-1, hIAP1, Hid, HIF1 α, HIP1, HIP116, HIPPI, HIPK1,2,3, histamine receptors, HIVEP1-3, HIV-EP1, HLTF, HM85, HM89, HM145, HMR, HNRPD, HRD1, Hrk, HtrA2, Huntingtin, HVEM, HVEML, HYP, IAP-1, IAP1, IAP2, IAP, iAPP, ICAD, ICBP90, ICE, ICEBERG, ICE-LAP3, ICE-LAP6, ICErel-II, ICErel-III, Ich1, ICH-1, Ich2, ICH-2, Ich3, ICH-3, ICOS, I-TRAF, I-FLICE, IEX-1m IFI, IFIT-1, IFIT-2, IFIT-3, IFIT-4, IFP35, IgE Fc receptor, IGF1 and its receptor, IGFBP-3, IKAP, Ikaros, IKB-1, IkB-a, IkB-b, IkB-e, IKKAP1, IKK-1, IKK-2, IKK-a, IKK-b, IKKg, interleukins, interleukin receptors, IL1 antagonist, anti-IL1, IL1RacP, IL8R1, ILA, ILC, ILP, ILP-1, ILP-2, ILT1-11, ING1, ING2, ING3, Inhibin, INK4, INK4A, integrin, IP10, INP10, IP30, Ipaf, IRAK, IAK2, IRAM-M, IRE1, RE1p, IRE, IRF, IRTA1-5, ISGF3g, ITA, It, Jab1, Jak1, 2, 3, JDP2, JIK, JN, K, K13, KARAP, KBF-1, KBF-2, KBF-3, KDS, KE05, KET, kf-1, KIAP, Killer, KIR2DL1-5, KIR2DS1-6, KROX2, L-Myc, lactalbumin α, LAG1, LAIR1, LALBA, LAM, LAP1, LAP3, LAR, LARD, LARC, LATS1, 2, LBP, Lck, LCP2, LD78b, LEFTY, LESTR, Leu1, Leu8, Leu14, leukotactin, LFA3, LFG, LICE, LICE2, LIF, LIGHT, LIR1, LIR-2, LIR-3, LIR-4, LIR-5, LIR-6, LIR-7, LIR-8, Livin, LMP1, LMW5-HL, LOK, Lot1, LRDD, LRP, Low affinity NGFR, LTa, LTb, LThR, LTP2, Ly63, lymphotactin, Ly1, Lyf1, Lysozyme, Lyt-10, LYVE1, LZK, M11; M159L, M160L, MA-3, MACH, Mad, Mad3, N4ADD, Maf, c-Maf, makorin, MAL, MALT, MAP-1, MAPKKKKs, MAPKKKs, MAPKKs, MAPKs, Math1, Max, MBD4, MBLR, MBP1, MCL1, Mch2, Mch3, Mch4, Mch5, Mch6, MCP1, MCP2, MCP3, Mda-7, MD-1, MD-2, Mdm2, Mdm4, MdmX, MDP62, mE10, MEF2a, MEKKs, Mel-18, MEMD, Meprin, metacaspase, MIC1, MID1, MIF, MIG, MIHC, MIP1-2-2a-2b, MIP-T3, MIR, MIS, MITF, MKK6, MKL1, MKP1, ML-1, ML-IAP, MLN64, MLX, MMP-1, MMP-2, MMP-3, MMP-4, MMP-5, MMP-6, MMP-7, MMP-8, MMP-9, MMP-10, MMP-11, MMP-12, MMP-13, MMP-14, MMP-15, MMP-16, MNDA, MNT, Mob1, mod (mdg4), MORT1, MPIF1, 2, MRFP, MRIT, Msx1, Msx2, MTAP44, Mtd, mTOR, MUC1, MUC2, MUL, MURF-1-2-3, myp-nop30, MxA, MxB, Mxi1, Mxi2, MYAK, Myc, MyD88, MyD118, MYLK, myoblast city, N-Myc, NAF1, NAIP, NALP1, NALP2, NAP2, NBAK3, Nbk, NBS1, NCA, NCAM, NCC-1, NCC-2, NCC-3, NCC-4, NDG1, neural sphigomyelinase, neuralin, NEMO, neogenin, neurotactin, neurocan, NF-kB, NF-X1, NFATs, NFIL3, NFIL6, NFkB1, 2, NIP1, NIP2, NIP3, NIPK, NIK, Nix, NKAT1-9, NKX2-5, nNOS, Notch, NOD-1, NOD-2, nop30, Nor-1, NOS2, NOS2B, NOS3, Nov, Noxa, NP10, Np95, Npc2, NPY3R, Nr-CAM, NR3, NR13, Nr-13, NRAGE, NRIF1, nucleolin, Nur77, NY-REN-64, OCIF, ODF, ODFR, OIAS, ORF16, posteoprotegerin, OSX, OX40, OX40L, OPG, OPGL, Osi, osteonectin, osteopontin, p14, p16, p331NG1, p35, p38, p49, p49, p55, p52, p53, p53AIP1, p53DINP1, p55, p60, p62, p62Dok, p63, p65, p73, p75NTR, p84, p100, p105, p193, p202, PAC1, PACAP, PACT, PAF400, PAG-3, PAG608, PAK1, PAK2, PAK3, PAP1, PAR4, paracaspase, PARC, Park2, parkin, PARP, PAX-2, PAX-3, PAX-5, PAX-8, PBEF, PBP, PD1, PDGF, PEA15, Pellino, PERK, PERP, PEK, Pelle, PEX10, PF4, PGRP, PI3K, Pidd, PIK-1, PLAB, Plk, Plk3, PKC, PKR, PKY, PLAGL1, PLAIDD, PLA2, PLC, PLD, Pli, Pml, PMP41, POSH, PP1A, PP14, PP2Ca, PRKR, PRSS25, polycystin 1, porimin, PRG1, Prk, PRL, prolactin receptor, PS-1, PS-2, PSCA, PSMD-11, PSMD-12, PSMD-13, PSP-C, PSK, PSSALRE, PTEN, PTK1, PTPs, PTP1C, PTP2C, PTP1G, PTPL1, PU.1, puckered, Pum, Q2/2, Rac, RAI, RANTES, RAX, Rb, Relish, RELT, Raf, RANK, RANKL, RAIDD, RBBP6, RBQ1, Rcm, Reaper, RelA, relaxin H1, H2, H3, RelB, Requiem, RFP, RFPL-1-2-3, RGS, RhoA, RICK, RIG-G, Ro52, Ro 60 kDa, ROC-1, ROC-2, RORgamma, ROX, RIFF, RIP, RIP2, RIP3, RNM561, RNF, RP-8, RP8, RP105, Rpr, RRP5, RYBP, S9, S152, SAG, Salvador, SAP1, SAPK2A, Sara, SARP 1,2,3, Sav, Sca2, SCA-2, SCC-S2, SCF, SCDGF, SCM1-1a, Scythe, SDF1, selectin L-E-P, SENP1, SENP2, sentrin/SUMO-specific protease, SETA, SFRP1-2-3-4-5, SFTP2, SFTPC, SGK, SGL, SGN5, SH2D1A, SHP1, 2, Siah, SIMPL, SIP27, SIP18, SIR2, SIVA, SLC, SLK, SLP-65, SLP-76, SLUG, Smac, SMADs, SMARCA3, SMN, SMT 3A, B, 3C, SNAIL, SNF2L3, SODD, somatostatin, Son3, SOX9, SP5, SP-C, SPARC, sphigomyelinase, Smase, SPOP, SPP1, SPRK, Spatzle, SFRP1,2,5, SS-56, SSA, SSA1, SSA2, ST2L, stabilin 1-2, STATs, STCP1, STG6, STEP, STM-2, Stra3, STRICA, Substance P, SUMO1, survivin, SYK, SY, T cell receptor, T2BP, T6BP, TAB1, Tab2, Tabby, TACI, TACTILE, Tag7, tachykinin, TAJ, TAK1, Tak1, TALL-1, TANK, TAO1, TAO2, TARC, TBX1, TBX-2, TBX-3, TBX-4, TBX-10, TBX-18, TBX-19, TBX-20, TBX-21, TBX-22, TCA3, TCA-3, TC1, TC2, TCR, TCTP, TDAG51, TEAP, TECK, TEGT, TEL, (TEL1), TEL2 (TELb), telokin, TERF, TFT, TGb, TGFβ-1, TGFβ-2, TGFβ-3, THG1, THRa, Thy-1, TIA1, TIAP, TIEG, TIF1, TIFγ, TIL6, TIMP1-2-3, TIP49, Tip60, TIRAP, TIS, TLRs, TLS, TMS1, TNFa, TNFAIP3, A20, TNFAIP6, TNFb, TNF-C, TNFR1, TNFR2, TNFR-II, TNFRSF1-19, Toll, Tollo, Tollip, TONEBP, Toso, Tp44, TPL-2, TR3, TR2L, TRABID, TRADD, TRADE, TRAF1, TRAF1(Dm), TRAF2, TRAF2(Dm), TRAF3, TRAF4, TRAF5, TRAF6, TRAF6(Dm), TRAFamn, TRAIL, TRAIL-R2, TRAMP, TRANCE, TRC8, TRIAD1-3, TRIF, TRIM, TRIP15, TRF-1, TRF-2, TRF1, TRF2, traube, TRDL-1, TRG, TRH, TRICK2, TRIP, Tristetraproline, TROY, TRRAP, TSC-22, TSC-22R, TTRAP, Tube, TUCAN, TWEAK, TX, TXBP151, TY, Tyk, UBCH7BP, UL36, UL37, Ulp, Unc5, UNC5h3, Urinary, stone protein (SPP1), USP7, usurpin, uterophi, vasopressin, vav, vav1, vav2, vav3, vav-1, vav-2, vav-3, versican, vICA, VIAF1, vBcl-2, VEGI, VEGF, Ventroptin, VG-1, VG71, VHR, v-IAPs, VI, warts, Wengen, WIG1, WISP-1, 2, 3, Wnt, WSL-1, WT1, WW45, WWOX, XAF1, XAP4, XCL1, 2, XEDAR, XIAP1, xRI, xRII, XICE, XICEa, XICE, Yama, YopJ, YY1AF, Zac, Zac1, ZAP70, ZBP89, zf3, ZFP26, ZFP127, ZH-DR, ZNF-40, ZNF-124, ZNF-148, as TFs, ZNF-144, ZNF-147, ZNF-179, ZNTF-313, ZNF-364 as RING, ZIP-kinase, ZPR, 18 wheeler, 24.6K Glu/Pro-rich, 4-1BB, 4-1BBL, 4-1BB ligand and 53BP2, 7TM.

Examples of pro-survival proteins include, but are not limited to Bcl-2, Bcl-XL, Mcl-1 and A1.

Examples of PUFAs contemplated by the present invention include:

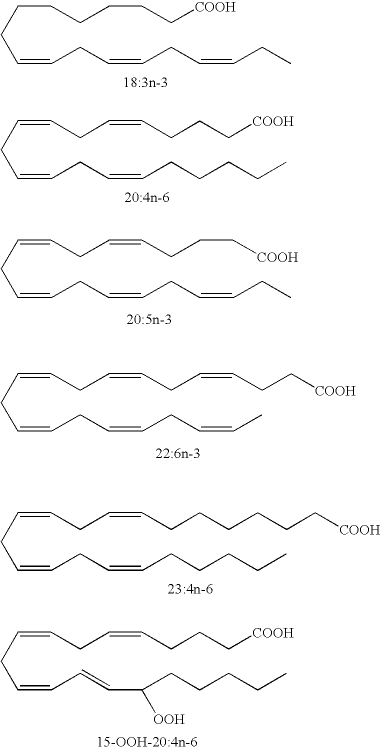

Natural PUFA and Hydroperoxy Derivative

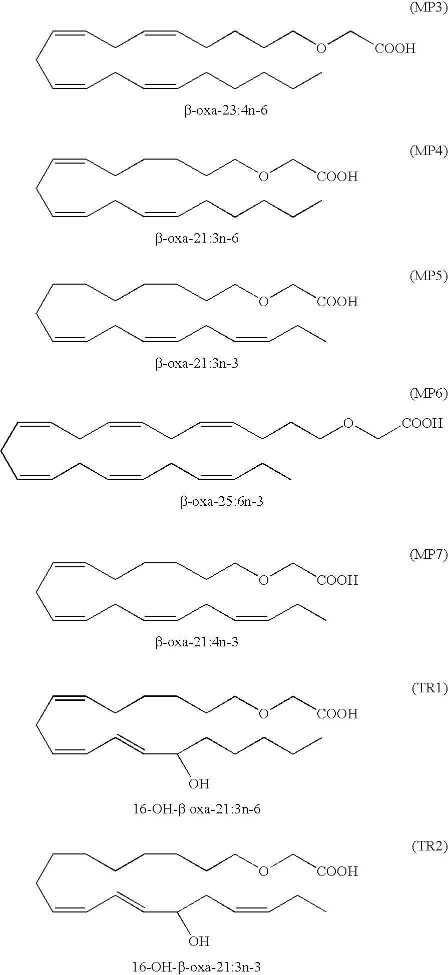

MP Series, β-Oxa Compounds

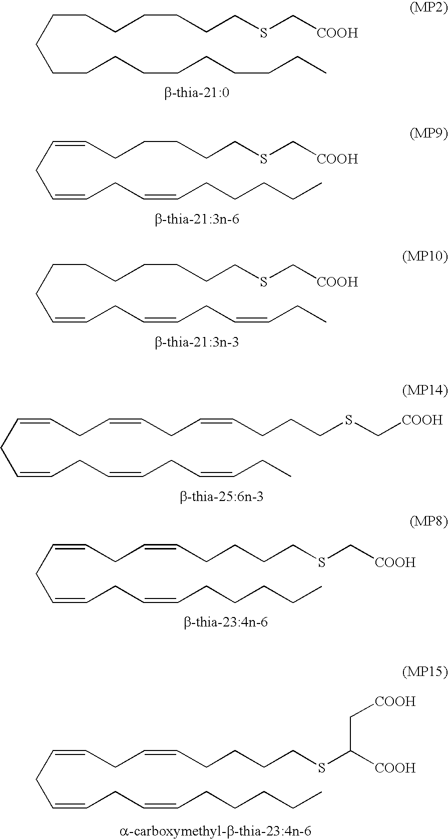

MP Series, β-Thia Compounds

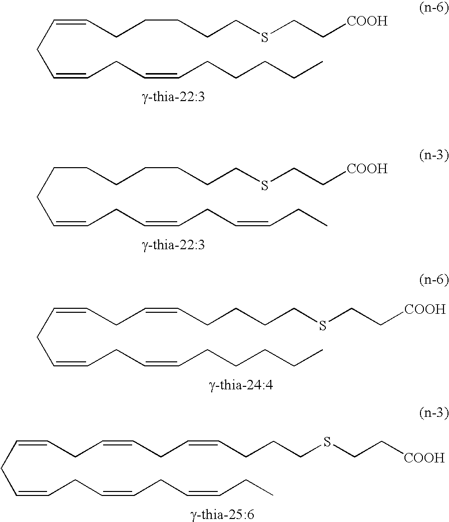

MP Series, γ-Thia Compounds

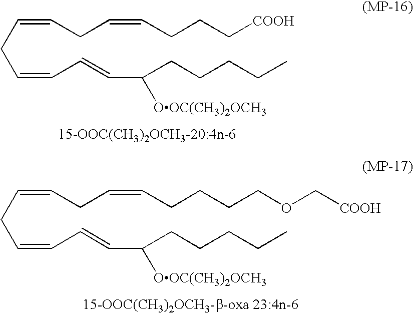

MP Series, Protected Hydroperoxy Compounds

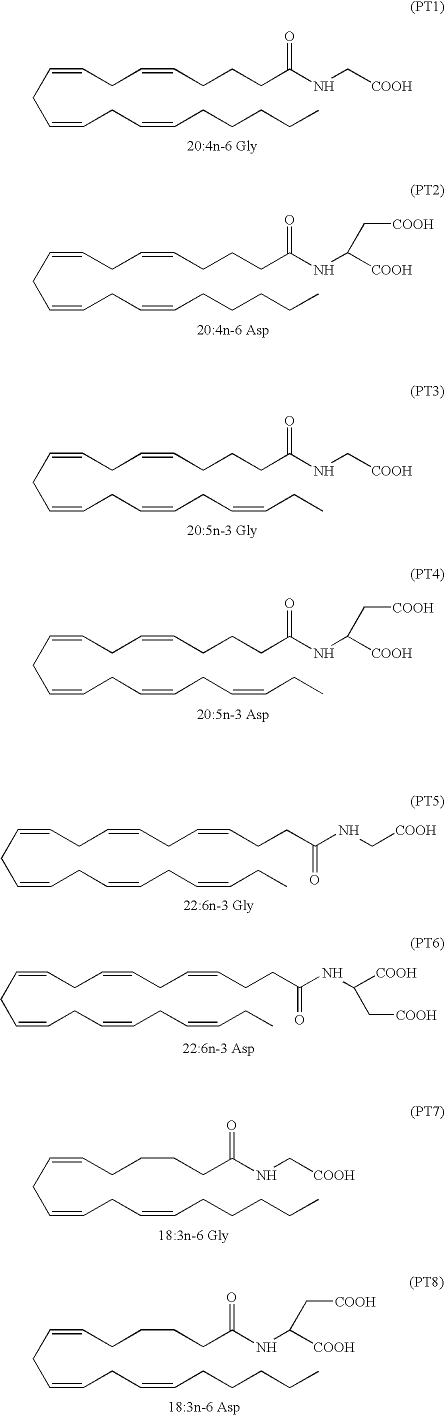

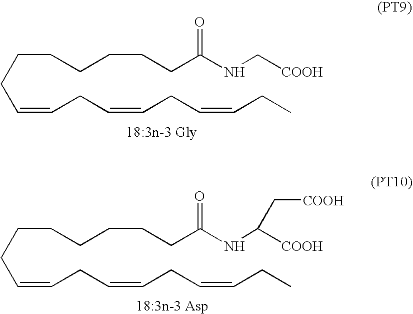

PT Series: PUFA-Amino Acid Conjugates

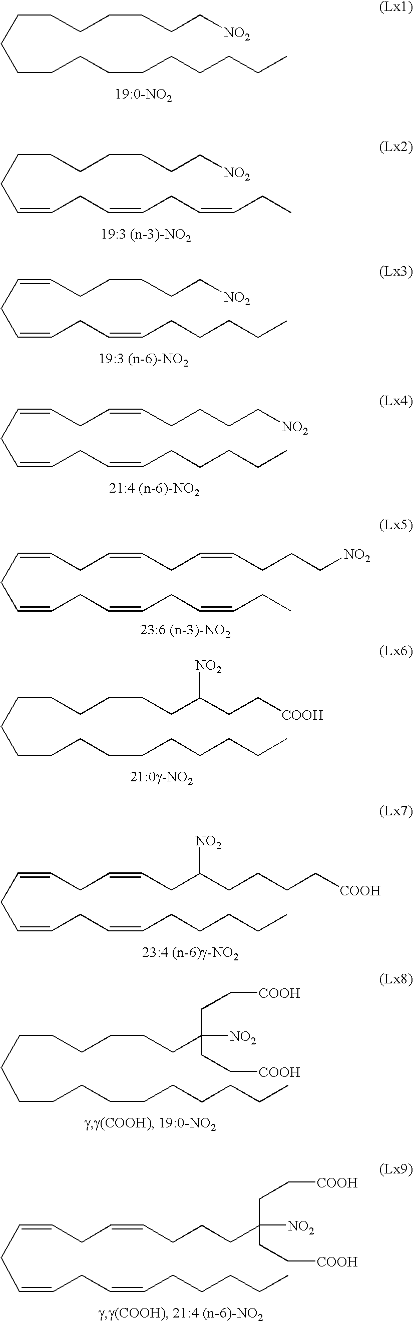

LX Series, Nitroanalogues of Fatty Acids

The present invention is directed inter alia to the treatment of pain, cancers, PKC- and/or NFκB-associated or -related conditions, vascular and/or immunological conditions, inflammatory conditions, neurological conditions and infection.

Other compounds contemplated by the present invention include β-oxa 23:0, β-thia 23:0, β-oxa 23:4 (n-6), β-oxa 21:3 (n-6); β-oxa 21:3 (n-3), β-oxa 25:6 (n-3), β-oxa 21:4 (n-3), β-thia 23:4 (n-6), β-thia 21:3 (n-6), β-thia 21:3 (n-3), γ-thia 24:4 (n-6), γ-thia 22:3 (n-6), γ-thia 22:3 (n-3), β-thia 25:6 (n-3), α-CH₂CO₂H-β-thia 23:4 (n-6), 15-OOCMe₂OMe 20:4 (n-6), 15-OOCMe₂OMe β-oxa 23:4 (n-6), 13-OH-β-oxa 21:3 (n-6), 13-OH-β-oxa 21:3 (n-3), 20:4 (n-6)-gly, 20:4 (n-6)-asp, 20:5 (n-3)-gly, 20:5 (n-3)-asp, 22:6 (n-3)-gly, 22:6 (n-3)-asp, 18:3 (n-6)-gly, 18:3 (n-6)-asp, 18:3 (n-3)-gly, 18:3 (n-3)-asp, 19:0-NO₂, 19:3 (n-3)-NO₂, 19:3 (n-6)-NO₂, 21:4 (n-6)-NO₂, 23:6 (n-3)-NO₂, γ-NO₂ 21:0, γ-NO₂ 23:4 (n-6) and γ,γ(COOH), 21:4 (n-6)NO₂.

The present invention is particularly directed to the treatment of pain including inter alia neuropathic or neurological pain, chronic pain, acute pain, migraine, headache inflammatory pain, post-operative pain, pain due to multiple sclerosis, Parkinson's disease or other neurological or autoimmune disorder or following or during periods of anxiety, delayed onset muscle soreness, burns or during or following infection or a convulsion, post-poliomyelitic pain, bipolar disorder, panic attack or epilepsy.

Neurological disease states which can be treated in accordance with the present invention include depression, including major depression (single episode, recurrent, melancholic), atypical, dysthymia, sub-syndromal, agitated, retarded, co-morbid with cancer, diabetes, or post-myocardial infarction, involutional, bipolar disorder, psychotic depression, endogenous and reactive, obsessive-compulsive disorder, or bulimia. In addition, NAALADase inhibitors can be used to treat patients suffering from pain (given alone or in combination with morphine, codeine, or dextroproposyphene), obsessive-compulsive personality disorder, post-traumatic stress disorder, hypertension, atherosclerosis, anxiety, anorexia nervosa, panic, social phobia, stuttering, sleep disorders, chronic fatigue, cognition deficit associated with Alzheimer's disease, alcohol abuse, appetite disorders, weight loss, agoraphobia, improving memory, amnesia, smoking cessation, nicotine withdrawal syndrome symptoms, disturbances of mood and/or appetite associated with pre-menstrual syndrome, depressed mood and/or carbohydrate craving associated with pre-menstrual syndrome, disturbances of mood, disturbances of appetite or disturbances which contribute to recidivism associated with nicotine withdrawal, circadian rhythm disorder, borderline personality disorder, hypochondriasis, premenstrual syndrome (PMS), late luteal phase dysphoric disorder, premenstrual dysphoric disorder, trichotillomania, symptoms following discontinuation of other anti-depressants, aggressive/intermittent explosive disorder, compulsive gambling, compulsive spending, compulsive sex, psychoactive substance use disorder, sexual disorder, schizophrenia, premature ejaculation, or psychiatric symptoms selected from stress, worry, anger, rejection sensitivity, and lack of mental or physical energy.

Other examples of pathological or psychological conditions which may be treated in accordance with this invention include, but are not limited to: Moderate Mental Retardation, Severe Mental Retardation, Profound Mental Retardation, Unspecified Mental Retardation, Autistic Disorder, Pervasive Development Disorder NOS, Attention-Deficit Hyperactivity Disorder, Conduct Disorder, Group Type, Conduct Disorder, Solitary Aggressive Type, Conduct Disorder, Undifferentiated Type, Tourettes Disorder, Chronic Motor or Vocal Tic Disorder, Transient Tic Disorder, Tic Disorder NOS, Primary Degenerative Dementia of the Alzheimer Type, Senile Onset, Uncomplicated, Primary Degenerative Dementia of The Alzheimer Type, Senile Onset, with Delirium, Primary Degenerative Dementia of the Alzheimer Type, Senile Onset, with Delusions, Primary Degenerative Dementia of the Alzheimer Type, Senile Onset, with Depression, Primary Degenerative Dementia of the Alzheimer Type, Presenile Onset, Uncomplicated, Primary Degenerative Dementia of the Alzheimer Type, Presenile Onset, with Delirium, Primary Degenerative Dementia of the Alzheimer Type, Presenile Onset, with Delusions, Primary Degenerative Dementia of the Alzheimer Type, Presenile Onset, with Depression, Multi-infarct dementia, Uncomplicated, Multi-infarct dementia, with Delirium, Multi-infarct Dementia, with Delusions, Multi-infarct Dementia, with Depression, Senile Dementia NOS, Presenile Dementia NOS, Alcohol Withdrawal Delirium, Alcohol Hallucinosis, Alcohol Dementia Associated with Alcoholism, Amphetamine or Similarly Acting Sympathomimetic Intoxication, Amphetamine or Similarly Acting Sympathomimetic Delusional Disorder, Cannabis Delusional Disorder, Cocaine Intoxication, Cocaine Delirium, Cocaine Delusional Disorder, Hallucinogen Hallucinosis, Hallucinogen Delusional Disorder, Hallucinogen Mood Disorder, Hallucinogen Posthallucinogen Perception Disorder, Phencyclidine (PCP or Similarly Acting Arylcyclohexylamine Intoxication, Phencyclidine (PCP) or Similarly Acting Arylcyclohexylamine Delirium, Phencyclidine (PCP) or Similarly Acting Arylcyclohexylamine Delusional. Disorder, Phencyclidine (PCP) or Similarly Acting Arylcyclohexylamine Hood Disorder, Phencyclidine (PCP) or Similarly Acting Arylcyclohexylamine Organic Mental Disorder NOS, Other or unspecified Psychoactive Substance Intoxication, Other or Unspecified Psychoactive Substance Delirium, Other or Unspecified Psychoactive Substance Dementia, Other or Unspecified Psychoactive Substance Delusional Disorder. Other or Unspecified Psychoactive Substance Hallucinosis, Other or Unspecified Psychoactive Substance Mood Disorder, Other or Unspecified Psychoactive Substance Anxiety Disorder, Other or Unspecified Psychoactive Substance Personality Disorder, Other or Unspecified Psychoactive Substance Organic Mental Disorder NOS, Delirium, Dementia, Organic Delusional Disorder, Organic Hallucinosis, Organic Mood Disorder, Organic Anxiety Disorder, Organic Personality Disorder, Organic Mental Disorder, Obsessive Compulsive Disorder, Post-traumatic Stress Disorder, Generalized Anxiety Disorder, Anxiety Disorder NOS, Body Dysmorphic Disorder, Hypochondriasis (or Hypochondriacal Neurosis), Somatization Disorder, Undifferentiated Somatoform Disorder, Somatoform Disorder NOS, Intermittent Explosive Disorder, Kleptomania, Pathological Gambling, Pyromania, Trichotillomania and Impulse Control Disorder NOS.

Additional examples of pathological or psychological conditions which may be treated as described in this invention include Schizophrenia, Catatonic, Sub-chronic, Schizophrenia, Catatonic, Chronic, Schizophrenia, Catatonic, Sub-chronic with Acute Exacerbation, Schizophrenia, Catatonic, Chronic with Acute Exacerbation, Schizophrenia, Catatonic, in Remission, Schizophrenia, Catatonic, Unspecified, Schizophrenia, Disorganized, Chronic, Schizophrenia, Disorganized, Subchronic with Acute Exacerbation, Schizophrenia, Disorganized, Chronic with Acute Exacerbation, Schizophrenia, Disorganized, in Remission, Schizophrenia, Disorganized, Unspecified, Schizophrenia, Paranoid, Subchronic, Schizophrenia, Paranoid, Chronic, Schizophrenia, Paranoid, Sub-chronic with Acute Exacerbation, Schizophrenia, Paranoid, Chronic with Acute Exacerbation, Schizophrenia, Paranoid, in Remission, Schizophrenia, Paranoid, Unspecified, Schizophrenia, Undifferentiated, Sub-chronic, Schizophrenia, Undifferentiated, Chronic, Schizophrenia, Undifferentiated, Sub-chronic with Acute Exacerbation, Schizophrenia, Undifferentiated, Chronic with Acute Exacerbation, Schizophrenia, Undifferentiated, in Remission, Schizophrenia, Undifferentiated, Unspecified, Schizophrenia, Residual, Sub-chronic, Schizophrenia, Residual, Chronic, Schizophrenia, Residual, Subchronic with Acute Exacerbation, Schizophrenia, Residual, Chronic with Acute Exacerbation, Schizophrenia, Residual, in Remission, Schizophrenia, Residual, unspecified, Delusional (Paranoid) Disorder, Brief Reactive Psychosis, Schizophreniform Disorder, Schizoaffective Disorder, induced Psychotic Disorder, Psychotic Disorder NOS (Atypical Psychosis), Bipolar Disorder, Mixed, Severe, without Psychotic Features, Bipolar Disorder, Manic, Severe, without Psychotic Features, Bipolar Disorder, Depressed, Severe, without Psychotic Features, Bipolar Disorder, Mixed, with Psychotic Features, Bipolar Disorder, Manic, with Psychotic Features, Bipolar Disorder, Depressed, with Psychotic Features, Bipolar Disorder NOS, Major Depression, Single Episode, with Psychotic Features, Major Depression, Recurrent with Psychotic Features Personality Disorders, Paranoid Personality Disorders, Schizoid, Personality Disorders, Schizotypal, Personality Disorders, Anti-social, Personality Disorders and Borderline.

Anxiety disorders which may be treated in accordance with this invention include, but are not limited to Anxiety Disorders, Panic Disorder, Panic Disorder with Agoraphobia, Panic Disorder without Agoraphobia, Agoraphobia without History of Panic Disorders, Social Phobia, Simple Phobia, Organic Anxiety Disorder, Psychoactive Substance Anxiety Disorder, Separation Anxiety Disorder, Avoidant Disorder of Childhood or Adolescence, and Overanxious Disorder.

Reference to cardiovascular disease includes strokes and any condition of the systemic vasculature and includes atherosclerosis, chronic heart failure and general heart disease.

Other conditions contemplated herein include but are not limited to Adult Respiratory distress syndrome, A-β-Lipoproteinemia, A-V, A β-2-Microglobulin Amyloidosis, A-T, A1AD, A 1AT, Aagenaes, Aarskog syndrome, Aarskog-Scott Syndrome, Aase-smith syndrome, Aase Syndrome, AAT, Abderhalden-Kaufmann-Lignac Syndrome, Abdominal Muscle Deficiency Syndrome, Abdominal Wall Defect, Abdominal Epilepsy, Abdominal Migraine, Abductor Spasmodic Dysphonia, Abductor Spastic Dysphonia, Abercrombie Syndrome, blepharon-Macrostomia Syndrome, ABS, Absence of HPRT, Absence of Corpus Callosun Schinzel Typ, Absence Defect of Limbs Scalp and Skull, Absence of Menstruation Primar, Absence of HGPRT, Absorptive Hyperoxaluriaor Enteric, Abt-Letterer-Siwe Disease, ACADL, ACADM Deficiency, ACADM, ACADS, Acanthocytosis-Neurologic Disorder, Acanthocytosis, Acantholysis Bullosa, Acanthosis Nigricans, Acanthosis Bullosa, Acanthosis Nigricans With Insulin Resistance Type A, Acanthosis Nigricans With Insulin Resistance Type B, Acanthotic Nevus, Acatalasemia, Acatalasia, ACC, Accessory Atrioventricular Pathways, Accessory Atrioventricular. Pathways, Acephaly, ACF with Cardiac Defects, Achalasia, Achard-Thiers Syndrome, ACHARD (Marfan variant), Achard's syndrome, Acholuric Jaundice, Achondrogenesis, Achondrogenesis Type IV, Achondrogenesis Type III, Achondroplasia, Achondroplasia Tarda, Achondroplastic Dwarfism, Achoo Syndrome, Achromat, Achromatope, Achromatopic, Achromatopsia, Achromic Nevi, Acid Ceramidase Deficiency, Acid Maltase Deficiency, Acid β-glucosidase Deficiency, Acidemia Methylmalonic, Acidemia Propionic, Acidemia with Episodic Ataxia and Weakness, Acidosis, Aclasis Tarsoepiphyseal, ACM, Acoustic Neurilemoma, Acoustic Neuroma, ACPS with Leg Hypoplasia, ACPS II, ACPS IV, ACPS III, Acquired Aphasia with Convulsive Disorder, Acquired Brown Syndrome, Acquired Epileptic Aphasia, Acquired Factor XIII Deficiency, Acquired Form of ACC (caused by infection while still in womb), Acquired Hyperoxaluria, Acquired Hypogammaglobulinemia, Acquired Immunodeficiency Syndrome (AIDS), Acquired Iron Overload, Acquired Lipodystrophy, Acquired Partial Lipodystrophy, Acquired Wandering Spleen, ACR, Acral Dysostosis with Facial and Genital Abnormalities, Acro Renal, Acrocallosal Syndrome Schinzel Type, Acrocephalosyndactyly, Acrocephalosyndactyly Type I, Acrocephalosyndactyly Type I Subtype I, Acrocephalopolysyndactyly Type II, Acrocephalopolysyndactyly Type III, Acrocephalopolysyndactyly Type IV, Acrocephalosyndactyly V (ACS5 or ACS V) Subtype I, Acrocephaly Skull Asymmetry and Mild Syndactyly, Acrocephaly, Acrochondrohyperplasia, Acrodermatitis Enteropathica, Acrodysostosis, Acrodystrophic Neuropathy, Acrofacial Dysostosis Nager Type, Acrofacial Dysostosis Postaxial Type, Acrofacial Dysostosis Type Genee-Wiedep, Acrogeria Familial, Acromegaly, Acromelalgia Hereditary, Acromesomelic Dysplasia, Acromesomelic Dwarfism, Acromicric Skeletal Dysplasia, Acromicric Dysplasia, Acroosteolysis with Osteoporosis and Changes in Skull and Mandible, Acroosteolysis, Acroparesthesia, ACS I, ACS Type II, ACS Type III, ACS, ACS3, ACTH Deficiency, Action Myoclonus, Acute Brachial Neuritis Syndrome, Acute Brachial Radiculitis Syndrome, Acute Cerebral Gaucher Disease, Acute Cholangitis, Acute Disseminated Encephalomyeloradiculopathy, Acute Disseminated Histiocytosis-X, Acute Hemorrhagic Polioencephalitis, Acute Idiopathic Polyneuritis, Acute Immune-Mediation Polyneuritis, Acute Infantile Pelizaeus-Merzbacher Brain Sclerosis, Acute Intermittant Porphyria, Acute Porphyrias, Acute Sarcoidosis, Acute Shoulder Neuritis, Acute Toxic Epidermolysis, Acyl-CoA Dehydrogenase Deficiency Long-Chain, Acyl-CoA Dehydrogenase Deficiency Short-Chain, Acyl-CoA Dihydroxyacetone Acyltransferase, Acyl-coenzyme A Oxidase Deficiency, ADA, ADA Deficiency, Adam Complex, Adamantiades-Behcet's Syndrome, Adamantinoma, Adams Oliver Syndrome, Adaptive Colitis, ADD combined type, ADD, Addison Disease with Cerebral Sclerosis, Addison's Anemia, Addison's Disease, Addison-Biermer Anemia, Addison-Schilder Disease, Addisonian Pernicious Anemia, Adducted Thumbs-Mental Retardation, Adductor Spasmodic Dysphonia, Adductor Spastic Dysphonia, Adenoma Associated Virilism of Older Women, Adenomatosis of the Colon and Rectum, Adenomatous polyposis of the Colon, Adenomatous Polyposis Familial, Adenosine Deaminase Deficiency, Adenylosuccinase deficiency, ADHD predominantly hyperactive-impulsive type, ADHD predominantly inattentive type, ADHD, Adhesive Arachnoiditis, Adie Syndrome, Adie's Syndrome, Adie's Tonic Pupil, Adie's Pupil, Adipogenital Retinitis Pigmentosa Polydactyly, Adipogenital-Retinitis Pigmentosa Syndrome, Adiposa Dolorosa, Adiposis Dolorosa, Adiposogenital Dystrophy, Adolescent Cystinosis, ADPKD, Adrenal Cortex Adenoma, Adrenal Disease, Adrenal Hyperfunction resulting from Pituitary ACTH Excess, Adrenal Hypoplasia, Adrenal Insufficiency, Adrenal Neoplasm, Adrenal Virilism, Adreno-Retinitis Pigmentosa-Polydactyly Syndrome, Adrenocortical Insufficiency, Adrenocortical Hypofunction, Adrenocorticotropic Hormone Deficiency Isolated, Adrenogenital Syndrome, Adrenoleukodystrophy, Adrenomyeloneuropathy, Adreno-Retinitis Pigmentosa-Polydactyly Syndrome, Adult Cystinosis, Adult Dermatomyositis, Adult Hypophosphatasia, Adult Macula Lutea Retinae Degeneration, Adult Onset ALD, Adult-Onset Ceroidosis, Adult Onset Medullary Cystic Disease, Adult Onset Pernicious Anemia, Adult Onset Schindler Disease, Adult-Onset Subacute Necrotizing Encephalomyelopathy, Adult Polycystic Kidney Disease, Adult Onset Medullary Cystic Disease, Adynlosuccinate Lyase Deficiency, AE, AEC Syndrome, AFD, Afibrinogenemia, African Siderosis, AGA, Aganglionic Megacolon, Age Related Macular Degeneration, Agenesis of Commissura Magna Cerebri, Agenesis of Corpus Callosum, Agenesis of Corpus Callosum-Infantile Spasms-Ocular Anomalies, Agenesis of Corpus Callosum and Chorioretinal Abnormality, Agenesis of Corpus Callosum-Chorioretinitis Abnormality, Aggressive mastocytosis, Agnosis Primary, AGR Triad, AGU, Agyria, Agyria-pachygria-band spectrum, AHC, AHD, AHDS, AHF Deficiency, AHG Deficiency, AHO, Ahumada Del Castillo, Aicardi Syndrome, AIED, AIMP, AIP, AIS, Akinetic Seizure, ALA-D Porphyria, Alactasia, Alagille Syndrome, Aland Island Eye Disease (X-Linked), Alaninuria, Albers-Schonberg Disease, Albinism, Albinismus, Albinoidism, Albright Hereditary Osteodystrophy, Alcaptonuria, Alcohol-Related Birth Defects, Alcoholic Embryopathy, Ald, ALD, ALD, Aldosterone, Aldosteronism With Normal Blood Pressure, Aldrich Syndrome, Alexander's Disease, Alexanders Disease, Algodystrophy, Algoneurodystrophy, Alkaptonuria, Alkaptonuric Ochronosis, Alkyl DHAP synthase deficiency, Allan-Herndon-Dudley Syndrome, Allan-Herndon Syndrome, Allan-Herndon-Dudley Mental Retardation, Allergic Granulomatous Antitis, Allergic Granulomatous Angiitis of Cronkhite-Canada, Alobar Holoprosencephaly, Alopecia Areata, Alopecia Celsi, Alopecia Cicatrisata, Alopecia Circumscripta, Alopecia-Poliosis-Uveitis-Vitiligo-Deafness-Cutaneous-Uveo-O, Alopecia Seminuniversalis, Alopecia Totalis, Alopecia Universalis, Alpers Disease, Alpers Diffuse Degeneration of Cerebral Gray Matter with Hepatic Cirrhosis, Alpers Progressive Infantile Poliodystrophy, Alpha-1-Antitrypsin Deficiency, α-1 4 Glucosidase Deficiency, α-Galactosidase A Deficiency, α-Galactosidase B Deficiency, a High-Density Lipoprotein Deficiency, α-L-Fucosidase Deficiency Fucosidosis Type 3, α-GalNAc Deficiency Schindler Type, Alphalipoproteinemia, Alpha Mannosidosis, α-N-Acetylgalactosaminidase Deficiency Schindler Type, α-NAGA Deficiency Schindler Type, α-Neuraminidase Deficiency, α-Thalassemia/mental retardation syndrome non-deletion type, α-lipoproteinemia, Alport Syndrome, ALS, Alstroem's Syndrome, Alstroem, Alstrom Syndrome, Alternating Hemiplegia Syndrome, Alternating Hemiplegia of Childhood, Alzheimer's Disease, Amaurotic Familial Idiocy, Amaurotic Familial Idiocy Adult, Amaurotic Familial Infantile Idiocy, Ambiguous Genitalia, AMC, AMD, Ameloblastoma, Amelogenesis Imperfecta, Amenorrhea-Galactorrhea Nonpuerperal, Amenorrhea-Galactorrhea-FSH Decrease Syndrome, Amenorrhea, Amino Acid Disorders, Aminoaciduria-Osteomalacia-Hyperphosphaturia Syndrome, AMN, Amniocentesis, Amniotic Bands, Amniotic Band Syndrome, Amniotic Band Disruption Complex, Amniotic Band Sequence, Amniotic Rupture Sequence, Amputation Congenital, AMS, Amsterdam Dwarf Syndrome de Lange, Amylo-1 6-Glucosidase Deficiency, Amyloid Arthropathy of Chronic Hemodialysis, Amyloid Corneal Dystrophy, Amyloid Polyneuropathy, Amyloidosis, Amyloidosis of Familial Mediterranean Fever, Amylopectinosis, Amyoplasia Congenita, Amyotrophic Lateral Sclerosis, Amyotrophic Lateral Sclerosis, Amyotrophic Lateral Sclerosis-Polyglucosan Bodies, AN, AN 1, AN 2, Anal Atresia, Anal Membrane, Anal Rectal Malformations, Anal Stenosis, Analine 60 Amyloidosis, Analphalipoproteinemia, Analrectal, Analrectal, Anaplastic Astrocytoma, Andersen Disease, Anderson-Fabry Disease, Andersen Glycogenosis, Anderson-Warburg Syndrome, Andre Syndrome, Andre Syndrome Type II, Androgen Insensitivity, Androgen Insensitivity Syndrome Partial, Androgen Insensitivity Syndrome Partial, Androgenic Steroids, Anemia Autoimmune Hemolytic, Anemia Blackfan Diamond, Anemia, Congenital, Triphalangeal Thumb Syndrome, Anemia Hemolytic Cold Antibody, Anemia Hemolytic with PGK Deficiency, Anemia Pernicious, Anencephaly, Angelman Syndrome, Angio-Osteohypertrophy Syndrome, Angiofollicular Lymph Node Hyperplasia, Angiohemophilia, Angiokeratoma Corporis, Angiokeratoma Corporis Diffusum, Angiokeratoma Diffuse, Angiomatosis Retina, Angiomatous Lymphoid, Angioneurotic Edema Hereditary, Anhidrotic Ectodermal Dysplasia, Anhidrotic X-Linked Ectodermal Dysplasias, Aniridia, Aniridia-Ambiguous Genitalia-Mental Retardation, Aniridia Associated with Mental Retardation, Aniridia-Cerebellar Ataxia-Mental Deficiency, Aniridia Partial-Cerebellar Ataxia-Mental Retardation, Aniridia Partial-Cerebellar Ataxia-Oligophrenia, Aniridia Type I, Aniridia Type II, Aniridia-Wilms' Tumor Association, Aniridia-Wilms' Tumor-Gonadoblastoma, Ankyloblepharon-Ectodermal Defects-Cleft Lip/Palate, Ankylosing Spondylitis, Annular groves, Anodontia, Anodontia Vera, Anomalous Trichromasy, Anomalous Dysplasia of Dentin, Coronal Dentin Dysplasia, Anomic Aphasia, Anophthalmia, Anorectal, Anorectal Malformations, Anosmia, Anterior Bowing of the Legs with Dwarfism, Anterior Membrane Corneal Dystrophy, Anti-Convulsant Syndrome, Anti-Epstein-Barr Virus Nuclear Antigen (EBNA) Antibody Deficiency, Antibody Deficiency, Antibody Deficiency with near normal Immunoglobulins, Anti-hemophilic Factor Deficiency, Anti-hemophilic Globulin Deficiency, Anti-phospholipid Syndrome, Anti-phospholipid Antibody Syndrome, Anti-thrombin III Deficiency, Anti-thrombin III Deficiency Classical (Type I), Anti-trypsin Deficiency, Antley-Bixler Syndrome, Antoni's Palsy, Anxietas Tibialis, Aorta Arch Syndrome, Aortic and Mitral Atresia with Hypoplasic Left Heart Syndrome, Aortic Stenosis, Aparoschisis, APC, APECED Syndrome, Apert Syndrome, Aperts, Aphasia, Aplasia Axialis Extracorticales Congenital, Aplasia Cutis Congenita, Aplasia Cutis Congenita with Terminal Transverse Limb Defects, Aplastic Anemia, Aplastic Anemia with Congenital Anomalies, APLS, Apnea, Appalachian Type Amyloidosis, Apple Peel Syndrome, Apraxia, Apraxia Buccofacial, Apraxia Constructional, Apraxia Ideational, Apraxia Ideokinetic, Apraxia Ideomotor, Apraxia Motor, Apraxia Oculomotor, APS, Arachnitis, Arachnodactyly Contractural Beals Type, Arachnodactyly, Arachnoid Cysts, Arachnoiditis Ossificans, Arachnoiditis, Aran-Duchenne, Aran-Duchenne Muscular Atrophy, Are generative Anemia, Arginase Deficiency, Argininemia, Arginino Succinase Deficiency, Argininosuccinase Deficiency, Argininosuccinate Lyase Deficiency, Argininosuccinic-Acid Lyase-ASL, Argininosuccinic Acid Synthetase Deficiency, Argininosuccinic Aciduria, Argonz-Del Castillo Syndrome, Arhinencephaly, Armenian Syndrome, Arnold-Chiari Malformation, Arnold-Chiari Syndrome, ARPKD, Arrhythmic Myoclonus, Arrhythmogenic Right Ventricular Dysplasia, Arteriohepatic Dysplasia, Arteriovenous Malformation, Arteriovenous Malformation of the Brain, Arteritis Giant Cell, Arthritis, Arthritis Urethritica, Arthro-Dento-Osteodysplasia, Arthro-Ophthalmopathy, Arthrochalasis Multiplex Congenita, Arthrogryposis Multiplex Congenita, Arthrogryposis Multiplex Congenita, Distal, Type IIA, ARVD, Arylsulfatase-B Deficiency, AS, ASA Deficiency, Ascending Paralysis, ASD, Atrioseptal Defects, ASH, Ashermans Syndrome, Ashkenazi Type Amyloidosis, ASL Deficiency, Aspartylglucosaminuria, Aspartylglycosaminuria, Asperger's Syndrome, Asperger's Type Autism, Asphyxiating Thoracic Dysplasia, Asplenia Syndrome, ASS Deficiency, Asthma, Astrocytoma Grade I (Benign), Astrocytoma Grade II (Benign), Asymnmetric Crying Facies with Cardiac Defects, Asymmetrical septal hypertrophy, Asymptomatic Callosal Agenesis, AT, AT III Deficiency, AT III Variant IA, AT III Variant Ib, AT 3, Ataxia, Ataxia Telangiectasia, Ataxia with Lactic Acidosis Type II, Ataxia Cerebral Palsy, Ataxiadynamia, Ataxiophemia, ATD, Athetoid Cerebral Palsy, Atopic Eczema, Atresia of Esophagus with or without Tracheoesophageal Fistula, Atrial Septal Defects, Atrial Septal Defect Primum, Atrial and Septal and Small Ventricular Septal Defect, Atrial Flutter, Atrial Fibrillation, Atriodigital Dysplasia, Atrioseptal Defects, Atrioventricular Block, Atrioventricular Canal Defect, Atrioventricular Septal Defect, Atrophia Bulborum Hereditaria, Atrophic Beriberi, Atrophy Olivopontocerebellar, Attention Deficit Disorder, Attention Deficit Hyperactivity Disorder, Attenuated Adenomatous Polyposis Coli, Atypical Amyloidosis, Atypical Hyperphenylalaninemia, Auditory Canal Atresia, Auriculotemporal Syndrome, Autism, Autism Asperger's Type, Autism Dementia Ataxia and Loss of Purposeful Hand Use, Autism Infantile Autism, Autoimmune Addison's Disease, Autoimmune Hemolytic Anemia, Autoimmune Hepatitis, Autoimmune-Polyendocrinopathy-Candidias, Autoimmune Polyglandular Disease Type I, Autosomal Dominant Albinism, Autosomal Dominant Compelling Helioophthalmic Outburst Syndrome, Autosomal Dominant Desmin Distal myopathy with Late Onset, Autosomal Dominant EDS, Autosomal Dominant Emery-Dreifuss Muscular Dystrophy, Autosomal Dominant Keratoconus, Autosomal Dominant Pelizaeus-Merzbacher Brain Sclerosis, Autosomal Dominant Polycystic Kidney Disease, Autosomal Dominant Spinocerebellar Degeneration, Autosomal Recessive Agammaglobulinemia, Autosomal Recessive Centronuclear myopathy, Autosomal Recessive Conradi-Hunermann Syndrome, Autosomal Recessive EDS, Autosomal Recessive Emery-Dreifuss Muscular Dystrophy, Autosomal Recessive Forms of Ocular Albinism, Autosomal Recessive Inheritance Agenesis of Corpus Callosum, Autosomal Recessive Keratoconus, Autosomal Recessive Polycystic Kidney Disease, Autosomal Recessive Severe Combined Immunodeficiency, AV, AVM, AVSD, AWTA, Axilla Abscess, Axonal Neuropathy Giant, Azorean Neurologic Disease, B-K Mole Syndrome, Babinski-Froelich Syndrome, BADS, Baillarger's Syndrome, Balkan Disease, Baller-Gerold Syndrome, Ballooning Mitral Valve, Balo Disease Concentric Sclerosis, Baltic Myoclonus Epilepsy, Bannayan-Zonaria syndrome (BZS), Bannayan-Riley-Ruvalcaba syndrome, Banti's Disease, Bardet-Biedl Syndrome, Bare Lymphocyte Syndrome, Barlow's syndrome, Barraquer-Simons Disease, Barrett Esophagus, Barrett Ulcer, Barth Syndrome, Bartter's Syndrome, Basal Cell Nevus Syndrome, Basedow Disease, Bassen-Kornzweig Syndrome, Batten Disease, Batten-Mayou Syndrome, Batten-Spielmeyer-Vogt's Disease, Batten Turner Syndrome, Batten Turner Type Congenital myopathy, Batten-Vogt Syndrome, BBB Syndrome, BBB Syndrome (Opitz), BBB Syndrome, BBBG Syndrome, BCKD Deficiency, BD, BDLS, BE, Beals Syndrome, Beals Syndrome, Beals-Hecht Syndrome, Bean Syndrome, BEB, Bechterew Syndrome, Becker Disease, Becker Muscular Dystrophy, Becker Nevus, Beckwith Wiedemann Syndrome, Beckwith-Syndrome, Begnez-Cesar's Syndrome, Behcet's syndrome, Behcet's Disease, Behr 1, Behr 2, Bell's Palsy, Benign Acanthosis Nigricans, Benign Astrocytoma, Benign Cranial Nerve Tumors, Benign Cystinosis, Benign Essential Blepharospasm, Benign Essential Tremor, Benign Familial Hematuria, Benign Focal Amyotrophy, Benign Focal Amyotrophy of ALS, Benign Hydrocephalus, Benign Hypermobility Syndrome, Benign Keratosis Nigricans, Benign Paroxysmal Peritonitis, Benign Recurrent Hematuria, Benign Recurrent Intrahepatic Cholestasis, Benign Spinal Muscular Atrophy with Hypertrophy of the Calves, Benign Symmetrical Lipomatosis, Benign Tumors of the Central Nervous System, Berardinelli-Seip Syndrome, Berger's Disease, Beriberi, Berman Syndrome, Bernard-Horner Syndrome, Bernard-Soulier Syndrome, Besnier Prurigo, Best Disease, β-Alanine-Pyruvate Aminotransferase, β-Galactosidase Deficiency Morquio Syndrome, β-Glucuronidase Deficiency, β Oxidation Defects, β Thalassemia Major, β Thalassemia Minor, β-lipoprotein Deficiency, Bethlem myopathy, Beuren Syndrome, BH4 Deficiency, Biber-Haab-Dimmer Corneal Dystrophy, Bicuspid Aortic Valve, Biedl-Bardet, Bifid Cranium, Bifunctional Enzyme Deficiency, Bilateral Acoustic Neurofibromatosis, Bilateral Acoustic Neuroma, Bilateral Right-Sidedness Sequence, Bilateral Renal Agenesis, Bilateral Temporal Lobe Disorder, Bilious Attacks, Bilirubin Glucuronosyltransferase Deficiency Type I, Binder Syndrome, Binswanger's Disease, Binswanger's Encephalopathy, Biotinidase deficiency, Bird-Headed Dwarfism Seckel Type, Birth Defects, Birthmark, Bitemporal Forceps Marks Syndrome, Biventricular Fibrosis, Bjornstad Syndrome, B-K Mole Syndrome, Black Locks-Albinism-Deafness of Sensoneural Type (BADS), Blackfan-Diamond Anemia, Blennorrheal Idiopathic Arthritis, Blepharophimosis, Ptosis, Epicanthus Inversus Syndrome, Blepharospasm, Blepharospasm Benign Essential, Blepharospasm Oromandibular Dystonia, Blessig Cysts, BLFS, Blindness, Bloch-Siemens Incontinentia Pigmenti Melanoblastosis Cutis Linearis, Bloch-Siemens-Sulzberger Syndrome, Bloch-Sulzberger Syndrome, Blood types, Blood type A, Blood type B, Blood type AB, Blood type O, Bloom Syndrome, Bloom-Torre-Mackacek Syndrome, Blue Rubber Bleb Nevus, Blue Baby, Blue Diaper Syndrome, BD, BOD, BOFS, Bone Tumor-Epidermoid Cyst-Polyposis, Bonnet-Dechaume-Blanc. Syndrome, Bonnevie-Ulrich Syndrome, Book Syndrome, BOR Syndrome, BORJ, Borjeson Syndrome, Borjeson-Forssman-Lehmann Syndrome, Bowen Syndrome, Bowen-Conradi Syndrome, Bowen-Conradi Hutterite, Bowen-Conradi Type Hutterite Syndrome, Bowman's Layer, BPEI, BPES, Brachial Neuritis, Brachial Neuritis Syndrome, Brachial Plexus Neuritis, Brachial-Plexus-Neuropathy, Brachiocephalic Ischemia, Brachmann-de Lange Syndrome, Brachycephaly, Brachymorphic Type Congenital, Bradycardia, Brain Tumors, Brain Tumors Benign, Brain Tumors Malignant, Branched Chain α-Ketoacid Dehydrogenase Deficiency, Branched Chain Ketonuria I, Brancher Deficiency, Branchio-Oculo-Facial Syndrome, Branchio-Oto-Renal Dysplasia, Branchio-Oto-Renal Syndrome, Branchiooculofacial Syndrome, Branchiootic Syndrome, Brandt Syndrome, Brandywine Type Dentinogenesis Imperfecta, Brandywine type Dentinogenesis Imperfecta, Breast Cancer, BRIC Syndrome, Brittle Bone Disease, Broad β Disease, Broad Thumb Syndrome, Broad Thumbs and Great Toes Characteristic Facies and Mental Retardation, Broad Thumb-Hallux, Broca's Aphasia, Brocq-Duhring Disease, Bronze Diabetes, Bronze Schilder's Disease, Brown Albinism, Brown Enamel Hereditary, Brown-Sequard Syndrome, Brown Syndrome, BRRS, Brueghel Syndrome, Bruton's A γ-globulinemia Common, BS, BSS, Buchanan's Syndrome, Budd's Syndrome, Budd-Chiari Syndrome, Buerger-Gruetz Syndrome, Bulbospinal Muscular Atrophy-X-linked, Bulldog Syndrome, Bullosa Hereditaria, Bullous CIE, Bullous Congenital Ichthyosiform Erythroderma, Bullous Ichthyosis, Bullous Pemphigoid, Burkitt's Lymphoma, Burkitt's Lymphoma African type, Burkitt's Lymphoma Non-african type, BWS, Byler's Disease, C Syndrome, C1 Esterase Inhibitor Dysfunction Type II Angioedema, C1-INH, C1 Esterase Inhibitor Deficiency Type I Angioedema, C1NH, Cacchi-Ricci Disease, CAD, CADASIL, CAH, Calcaneal Valgus, Calcaneovalgus, Calcium Pyrophosphate Dihydrate Deposits, Callosal Agenesis and Ocular Abnormalities, Calves-Hypertrophy of Spinal Muscular Atrophy, Campomelic Dysplasia, Campomelic Dwarfism, Campomelic Syndrome, Camptodactyly-Cleft Palate-Clubfoot, Camptodactyly-Limited Jaw Excursion, Camptomelic Dwarfism, Camptomelic Syndrome, Camptomelic Syndrome Long-Limb Type, Camurati-Engelmann Disease, Canada-Cronkhite Disease, Canavan disease, Canavan's Disease Included, Canavan's Leukodystrophy, Cancer, Cancer Family Syndrome Lynch Type, Cantrell Syndrome, Cantrell-Haller-Ravich Syndrome, Cantrell Pentalogy, Carbamyl Phosphate Synthetase Deficiency, Carbohydrate Deficient Glycoprotein Syndrome, Carbohydrate-Deficient Glycoprotein Syndrome Type Ia, Carbohydrate-Induced Hyperlipemia, Carbohydrate Intolerance of Glucose Galactose, Carbon Dioxide Acidosis, Carboxylase Deficiency Multiple, Cardiac-Limb Syndrome, Cardio-auditory Syndrome, Cardioauditory Syndrome of Jervell and Lange-Nielsen, Cardiocutaneous Syndrome, Cardio-facial-cutaneous syndrome, Cardiofacial Syndrome Cayler Type, Cardiomegalia Glycogenica Diffusa, Cardiomyopathic Lentiginosis, Cardiomyopathy, Cardiomyopathy Associated with Desmin Storage myopathy, Cardiomyopathy Due to Desmin Defect, Cardiomyopathy-Neutropenia Syndrome, Cardiomyopathy-Neutropenia Syndrome Lethal Infantile Cardio myopathy, Cardiopathic Amyloidosis, Cardiospasm, Cardocardiac Syndrome, Carnitine-Acylcarnitine Translocase Deficiency, Carnitine Deficiency and Disorders, Carnitine Deficiency Primary, Carnitine Deficiency Secondary, Carnitine Deficiency Secondary to MCAD Deficiency, Carnitine Deficiency Syndrome, Carnitine Palmitoyl Transferase I & II (CPT I & II), Carnitine Palmitoyltransferase Deficiency, Carnitine Palmitoyltransferase Deficiency Type 1, Carnitine Palmitoyltransferase Deficiency Type 2 benign classical muscular form included severe infantile form included, Carnitine Transport Defect (Primary Carnitine Deficiency), Carnosinase Deficiency, Carnosinemia, Caroli Disease, Carpenter syndrome, Carpenter's, Cartilage-Hair Hypoplasia, Castleman's Disease, Castleman's Disease Hyaline Vascular Type, Castleman's Disease Plasma Cell Type, Castleman Tumor, Cat Eye Syndrome, Cat's Cry Syndrome, Catalayse deficiency, Cataract-Dental Syndrome, Cataract X-Linked with Hutchinsonian Teeth, Catecholamine hormones, Catel-Manzke Syndrome, Catel-Manzke Type Palatodigital Syndrome, Caudal Dysplasia, Caudal Dysplasia Sequence, Caudal Regression Syndrome, Causalgia Syndrome Major, Cavernomas, Cavernous Angioma, Cavernous Hemangioma, Cavernous Lymphangioma, Cavernous Malformations, Cayler Syndrome, Cazenave's Vitiligo, CBGD, CBPS, CCA, CCD, CCHS, CCM Syndrome, CCMS, CCO, CD, CDG1a, CDG1A, CDGS Type Ia, CDGS, CDI, CdLS, Celiac Disease, Celiac sprue, Celiac Sprue-Dermatitis, Cellular Immunodeficiency with Purine Nucleoside Phosphorylase Deficiency, Celsus' Vitiligo, Central Apnea, Central Core Disease, Central Diabetes Insipidus, Central Form Neurofibromatosis, Central Hypoventilation, Central Sleep Apnea, Centrifugal Lipodystrophy, Centronuclear myopathy, CEP, Cephalocele, Cephalothoracic Lipodystrophy, Ceramide Trihexosidase Deficiency, Cerebellar Agenesis, Cerebellar Aplasia, Cerebellar Hemiagenesis, Cerebellar Hypoplasia, Cerebellar Vermis Aplasia. Cerebellar Vermis Agenesis-Hypernea-Episodic Eye Moves-Ataxia-Retardation, Cerebellar Syndrome, Cerebellarparenchymal Disorder IV, Cerebellomedullary Malformation Syndrome, Cerebello-Oculocutaneous Telangiectasia, Cerebelloparenchymal Disorder IV Familial, Cerebellopontine Angle Tumor, Cerebral Arachnoiditis, Cerebral Autosomal Dominant Arteriopathy with Subcortical Infarcts and Leukodystrophy, Cerebral Beriberi, Cerebral Diplegia, Cerebral Gigantism, Cerebral Malformations Vascular, Cerebral Palsy, Cerebro-Oculorenal Dystrophy, Cerebro-Oculo-Facio-Skeletal Syndrome, Cerebrocostomandibular syndrome, Cerebrohepatorenal Syndrome, Cerebromacular Degeneration, Cerebromuscular Dystrophy Fukuyama Type, Cerebroocular Dysgenesis, Cerebroocular Dysplasia-Muscular Dystrophy Syndrome, Cerebrooculofacioskeletal Syndrome, Cerebroretinal Arteriovenous Aneurysm, Cerebroside Lipidosis, Cerebrosidosis, Cerebrotendinous Xanthomatosis, Cerebrovascular Ferrocalcinosis, Ceroid-Lipofuscinosis Adult form, Cervical Dystonia, Cervical Dystonia, Cervico-Oculo-Acoustic Syndrome, Cervical Spinal Stenosis, Cervical Vertebral Fusion, CES, CF, CFC syndrome, CFIDS, CFND, CGD, CGF, Chalasodermia Generalized, Chanarin Dorfman Disease, Chanarin Dorfman Syndrome, Chanarin Dorfman Ichthyosis Syndrome, Chandler's Syndrome, Charcot's Disease, Charcot-Marie-Tooth, Charcot-Marie-Tooth Disease, Charcot-Marie-Tooth Disease Variant, Charcot-Marie-Tooth-Roussy-Levy Disease, CHARGE Association, Charge Syndrome, CHARGE Syndrome, Chaund's Ectodermal Dysplasias, Chediak-Higashi Syndrome, Chediak-Steinbrinck-Higashi Syndrome, Cheilitis Granulomatosa, Cheiloschisis, Chemke Syndrome, Cheney Syndrome, Cherry Red Spot and Myoclonus Syndrome, CHF, CHH, Chiari's Disease, Chiari Malformation I, Chiari Malformation, Chiari Type I (Chiari Malformation I), Chiari Type II (Chiari Malformation II), Chiari I Syndrome, Chiari-Budd Syndrome, Chiari-Frommel Syndrome, Chiari Malformation II, CHILD Syndrome, CHILD Ichthyosis Syndrome, CHILD Syndrome Ichthyosis, Childhood Adrenoleukodystrophy, Childhood Dermatomyositis, Childhood-onset Dystonia, Childhood Cyclic Vomiting, Childhood Giant Axonal Neuropathy, Childhood Hypophosphatasia, Childhood Muscular Dystrophy, CHN, Cholestasis, Cholestasis Hereditary Norwegian Type, Cholestasis Intrahepatic, Cholestasis Neonatal, Cholestasis of Oral Contraceptive Users, Cholestasis with Peripheral Pulmonary Stenosis, Cholestasis of Pregnancy, Cholesterol Desmolase Deficiency, Chondrodysplasia Punctata, Chondrodystrophia Calcificans Congenita, Chondrodystrophia Fetalis, Chondrodystrophic Myotonia, Chondrodystrophy, Chondrodystrophy with Clubfeet, Chondrodystrophy Epiphyseal, Chondrodystrophy Hyperplastic Form, Chondroectodermal Dysplasias, Chondrogenesis Imperfecta, Chondrohystrophia, Chondroosteodystrophy, Choreoacanthocytosis, Chorionic Villi Sampling, Chorioretinal Anomalies, Chorioretinal Anomalies with ACC, Chorireninal Coloboma-Joubert Syndrome, Choroidal Sclerosis, Choroideremia, Chotzen Syndrome, Christ-Siemens-Touraine Syndrome, Christ-Siemans-Touraine Syndrome, Christmas Disease, Christmas Tree Syndrome, Chromosome 3 Deletion of Distal 3p, Chromosome 3 Distal 3p Monosomy, Chromosome 3-Distal 3q2 Duplication, Chromosome 3-Distal 3q2 Trisomy, Chromosome 3 Monosomy 3p2, Chromosome 3q Partial Duplication Syndrome, Chromosome 3q, Partial Trisomy Syndrome, Chromosome 3-Trisomy 3q2, Chromosome 4 Deletion 4q31-qter Syndrome, Chromosome 4 Deletion 4q32-qter Syndrome, Chromosome 4 Deletion 4q33-qter Syndrome, Chromosome 4 Long Arm Deletion, Chromosome 4 Long Arm Deletion, Chromosome 4 Monosomy 4q, Chromosome 4-Monosomy 4q, Chromosome 4 Monosomy Distal 4q, Chromosome 4 Partial Deletion 4p, Chromosome 4, Partial Deletion of the Short Arm, Chromosome 4 Partial Monosomy of Distal 4q, Chromosome 4 Partial Monosomy 4p, Chromosome 4 Partial Trisomy 4 (q25-qter), Chromosome 4 Partial Trisomy 4 (q26 or q27-qter), Chromosome 4 Partial Trisomy 4 (q31 or 32-qter), Chromosome 4 Partial Trisomy 4p, Chromosome 4 Partial Trisomies 4q2 and 4q3, Chromosome 4 Partial Trisomy Distal 4, Chromosome 4 Ring, Chromosome 4 4q Terminal Deletion Syndrome, Chromosome 4q-Syndrome, Chromosome 4q-Syndrome, Chromosome 4 Trisomy 4, Chromosome 4 Trisomy 4p, Chromosome 4 XY/47 XXY (Mosiac), Chromosome 5 Monosomy 5p, Chromosome 5, Partial Deletion of the Short Arm Syndrome, Chromosome 5 Trisomy 5p, Chromosome 5 Trisomy 5p Complete (5p11-pter), Chromosome 5 Trisomy 5p Partial (5p3 or 14-pter), Chromosome 5p-Syndrome, Chromosome 6 Partial Trisomy 6q, Chromosome 6 Ring, Chromosome 6 Trisomy 6q2, Chromosome 7 Monosomy 7p2, Chromosome 7 Partial Deletion of Short Arm (7p2-), Chromosome 7 Terminal 7p Deletion, Chromosome 8 Monosomy 8p2, Chromosome 8 Monosomy 8p21-pter, Chromosome 8 Partial Deletion (short arm), Chromosome 8 Partial Monosomy 8p2, Chromosome 9 Complete Trisomy 9P, Chromosome 9 Partial Deletion of Short Arm, Chromosome 9 Partial Monosomy 9p, Chromosome 9 Partial Monosomy 9p22, Chromosome 9 Partial Monosomy 9p22-pter, Chromosome 9 Partial Trisomy 9P Included, Chromosome 9 Ring, Chromosome 9 Tetrasomy 9p, Chromosome 9 Tetrasomy 9p Mosaicism, Chromosome 9 Trisomy 9p (Multiple Variants), Chromosome 9 Trisomy 9 (pter-p21 to q32), Chromosome 9 Trisomy Mosaic, Chromosome 9 Trisomy Mosaic, Chromosome 10 Distal Trisomy 10q, Chromosome 10 Monosomy, Chromosome 10 Monosomy 10p, Chromosome 10, Partial Deletion (short arm), Choromsome 10, 10p-Partial, Chromosome 10 Partial Trisomy 10q24-qter, Chromosome 10 Trisomy 10q2, Partial Monosomy of Long Arm of Chromosome 11, Chromosome 11 Partial Monosomy 11q, Chromosome 11 Partial Trisomy, Chromosome 11 Partial Trisomy 11q13-qter, Chromosome 11 Partial Trisomy 11q21-qter, Chromosome 11 Partial Trisomy 11q23-qter, Chromosome 11q, Partial Trisomy, Chromosome 12 Isochromosome 12p Mosaic, Chromosome 13 Partial Monosomy 13q, Chromosome 13, Partial Monosomy of the Long Arm, Chromosome 14 Ring, Chromosome 14 Trisomy, Chromosome 15 Distal Trisomy 15q, Chromosome r15, Chromosome 15 Ring, Chromosome 15 Trisomy 15q2, Chromosome 15q, Partial Duplication Syndrome, Chromosome 17 Interstitial Deletion 17p, Chromosome 18 Long Arm Deletion Syndrome, Chromosome 18 Monosomy 18p, Chromosome 18 Monosomy 18Q, Chromosome 18 Ring, Chromosome 18 Tetrasomy 18p, Chromosome 18q-Syndrome, Chromosome 21 Mosaic 21 Syndrome, Chromosome 21 Ring, Chromosome 21 Translocation 21 Syndrome, Chromosome 22 Inverted Duplication (22pter-22q11), Chromosome 22 Partial Trisomy (22pter-22q11), Chromosome 22 Ring, Chromosome 22 Trisomy Mosaic, Chromosome 48 XXYY, Chromosome 48 XXXY, Chromosome r15, Chromosomal Triplication, Chromosome Triplication, Chromosome Triploidy Syndrome, Chromosome X, Chromosome XXY, Chronic Acholuric Jaundice, Chronic Adhesive Arachnoiditis, Chronic Adrenocortical Insufficiency, Chronic Cavernositis, Chronic Congenital Aregenerative Anemia, Chronic Dysphagocytosis, Chronic Familial Granulomatosis, Chronic Familial Icterus, Chronic Fatigue Immune Dysfunction Syndrome (CFIDS), Chronic Granulomatous Disease, Chronic Guillain-Barre Syndrome, Chronic Idiopathic Jaundice, Chronic Idiopathic Polyneuritis (CIP), Chronic Inflammatory Demyelinating Polyneuropathy, Chronic Inflammatory Demyelinating Polyradiculoneuropathy, Chronic Motor Tic, Chronic Mucocutaneous Candidiasis, Chronic Multiple Tics, Chronic Non-Specific Ulcerative Colitis, Chronic Obliterative Cholangitis, Chronic Peptic Ulcer and Esophagitis Syndrome, Chronic Progressive Chorea, Chronic Progressive External Ophthalmoplegia Syndrome, Chronic Progressive External Ophthalmoplegia and myopathy, Chronic Progressive External Ophthalmoplegia with Ragged Red Fibers, Chronic Relapsing Polyneuropathy, Chronic Sarcoidosis, Chronic Spasmodic Dysphonia, Chronic Vomiting in Childhood, CHS, Churg-Strauss Syndrome, Cicatricial Pemphigoid, CIP, Cirrhosis Congenital Pigmentary, Cirrhosis, Cistinuria, Citrullinemia, CJD, Classic Schindler Disease, Classic Type Pfeiffer Syndrome, Classical Maple Syrup Urine Disease, Classical Hemophilia, Classical Form Cockayne Syndrome Type I (Type A), Classical Leigh's Disease, Classical Phenylketonuria, Classical X-Linked Pelizaeus-Merzbacher Brain Sclerosis, CLE, Cleft Lip/Palate Mucous Cysts Lower Lip PP Digital and Genital Anomalies, Cleft Lip-Palate Blepharophimosis Lagopthalmos and Hypertelorism, Cleft Lip/Palate with Abnormal Thumbs and Microcephaly, Cleft palate-joint contractures-dandy walker malformations, Cleft Palate and Cleft Lip, Cleidocranial Dysplasia w/ Micrognathia, Absent Thumbs, & Distal Aphalangia, Cleidocranial Dysostosis, Cleidocranial Dysplasia, Click murmur syndrome, CLN1, Clonic Spasmodic, Cloustons Syndrome, Clubfoot, CMDI, CMM, CMT, CMTC, CMTX, COA Syndrome, Coarctation of the aorta, Coats' Disease, Cobblestone dysplasia, Cochin Jewish Disorder, Cockayne Syndrome, COD-MD Syndrome, COD, Coffin Lowry Syndrome, Coffin Syndrome, Coffin Siris Syndrome, COFS Syndrome, Cogan Corneal Dystrophy, Cogan Reese Syndrome, Cohen Syndrome, Cold Agglutinin Disease, Cold Antibody Disease, Cold Antibody Hemolytic Anemia, Colitis Ulcerative, Colitis Gravis, Colitis Ulcerative Chronic Non-Specific Ulcerative Colitis, Collodion Baby, Coloboma Heart Defects Atresia of the Choanae Retardation of Growth and Development Genital and Urinary Anomalies and Ear Anomalies, Coloboma, Colonic Neurosis, Color blindness, Colour blindness, Colpocephaly, Columnar-Like Esophagus, Combined Cone-Rod Degeneration, Combined Immunodeficiency with Immunoglobulins, Combined Mesoectodermal Dysplasia, Common Variable Hypogammaglobulinemia, Common Variable Immunodeficiency, Common Ventricle, Communicating Hydrocephalus, Complete Absence of Hypoxanthine-Guanine Phosphoribosyltransferase, Complete Atrioventricular Septal Defect, Complement Component 1 Inhibitor Deficiency, Complement Component C1 Regulatory Component Deficiency, Complete Heart Block, Complex Carbohydrate Intolerance, Complex Regional Pain Syndrome, Complex V ATP Synthase Deficiency, Complex I, Complex I NADH dehydrogenase deficiency, Complex II, Complex II Succinate dehydrogenase deficiency, Complex III, Complex III Ubiquinone-cytochrome c oxidoreductase deficiency, Complex IV, Complex IV Cytochrome C Oxidase Deficiency, Complex IV Deficiency, Complex V, Cone-Rod Degeneration, Cone-Rod Degeneration Progressive, Cone Dystrophy, Cone-Rod Dystrophy, Confluent Reticular Papillomatosis, Congenital with low PK Kinetics, Congenital Absence of Abdominal Muscles, Congenital Absence of the Thymus and Parathyroids, Congenital Achromia, Congenital Addison's Disease, Congenital Adrenal Hyperplasia, Congenital Adreneal Hyperplasia, Congenital Afibrinogenemia, Congenital Alveolar Hypoventilation, Congenital Anemia of Newborn, Congenital Bilateral Persylvian Syndrome, Congenital Brown Syndrome, Congenital Cardiovascular Defects, Congenital Central Hypoventilation Syndrome, Congenital Cerebral Palsy, Congenital Cervical Synostosis, Congenital Clasped Thumb with Mental Retardation, Congenital Contractural Arachnodactyly, Congenital Contractures Multiple with Arachnodactyly, Congenital Cyanosis, Congenital Defect of the Skull and Scalp, Congenital Dilatation of Intrahepatic Bile Duct, Congenital Dysmyelinating Neuropathy, Congenital Dysphagocytosis, Congenital Dysplastic Angiectasia, Congenital Erythropoietic Porphyria, Congenital Factor XIII Deficiency, Congenital Failure of Autonomic Control of Respiration, Congenital Familial Nonhemolytic Jaundice Type I, Congenital Familial Protracted Diarrhea, Congenital Form Cockayne Syndrome Type II (Type B), Congenital Generalized Fibromatosis, Congenital German Measles, Congenital Giant Axonal Neuropathy, Congenital Heart Block, Congenital Heart Defects, Congenital Hemidysplasia with Ichthyosis Erythroderma and Limb Defects, Congenital Hemolytic Jaundice, Congenital Hemolytic Anemia, Congenital Hepatic Fibrosis, Congenital Hereditary Corneal Dystrophy, Congenital Hereditary Lymphedema, Congenital Hyperchondroplasia, Congenital Hypomyelinating Polyneuropathy, Congenital Hypomyelination Neuropathy, Congenital Hypomyelination, Congenital Hypomyelination (Onion Bulb) Polyneuropathy, Congenital Ichthyosiform Erythroderma, Congenital Keratoconus, Congenital Lactic Acidosis, Congenital Lactose Intolerance, Congenital Lipodystrophy, Congenital Liver Cirrhosis, Congenital Lobar Emphysema, Congenital Localized Emphysema, Congenital Macroglossia, Congenital Medullary Stenosis, Congenital Megacolon, Congenital Melanocytic Nevus, Congenital Mesodermal Dysmorphodystrophy, Congenital Mesodermal Dystrophy, Congenital Microvillus Atrophy, Congenital Multiple Arthrogryposis, Congenital Myotonic Dystrophy, Congenital Neuropathy caused by Hypomyelination, Congenital Pancytopenia, Congenital Pernicious Anemia, Congenital Pernicious Anemia due to Defect of Intrinsic Factor, Congenital Pernicious Anemia due to Defect of Intrinsic Factor, Congenital Pigmentary Cirrhosis, Congenital Porphyria, Congenital Proximal myopathy Associated with Desmin Storage myopathy, Congenital Pulmonary Emphysema, Congenital Pure Red Cell Anemia, Congenital Pure Red Cell Aplasia, Congenital Retinal Blindness, Congenital Retinal Cyst, Congenital Retinitis Pigmentosa, Congenital Retinoschisis, Congenital Rod Disease, Congenital Rubella Syndrome, Congenital Scalp Defects with Distal Limb Reduction Anomalies, Congenital Sensory Neuropathy, Congenital SMA with arthrogryposis, Congenital Spherocytic Anemia, Congenital Spondyloepiphyseal Dysplasia, Congenital Tethered Cervical Spinal Cord Syndrome, Congenital Tyrosinosis, Congenital Varicella Syndrome, Congenital Vascular Cavernous Malformations, Congenital Vascular Veils in the Retina, Congenital Word Blindness, Congenital Wandering Spleen (Pediatric), Congestive Cardio myopathy, Conical Cornea, Conjugated Hyperbilirubinemia, Conjunctivitis, Conjunctivitis Ligneous, Conjunctivo-Urethro-Synovial Syndrome, Conn's Syndrome, Connective Tissue Disease, Conradi Disease, Conradi Hunermann Syndrome, Constitutional Aplastic Anemia, Constitutional Erythroid Hypoplasia, Constitutional Eczema, Constitutional Liver Dysfunction, Constitutional Thrombopathy, Constricting Bands Congenital, Constrictive Pericarditis with Dwarfism, Continuous Muscle Fiber Activity Syndrome, Contractural Arachnodactyly, Contractures of Feet Muscle Atrophy and Oculomotor Apraxia, Convulsions, Cooley's anemia, Copper Transport Disease, Coproporphyria Porphyria Hepatica, Cor Triatriatum, Cor Triatriatum Sinistrum, Cor Triloculare Biatriatum, Cor Biloculare, Cori Disease, Cornea Dystrophy, Corneal Amyloidosis, Corneal Clouding-Cutis Laxa-Mental Retardation, Corneal Dystrophy, Cornelia de Lange Syndrome, Coronal Dentine Dysplasia, Coronary Artery Disease, Coronary Heart Disease, Corpus Callosum Agenesis, Cortical-Basal Ganglionic Degeneration, Corticalis Deformaris, Cortico-Basal Ganglionic Degeneration (CBGD), Corticobasal Degeneration, Corticosterone Methyloxidase Deficiency Type I, Corticosterone Methyloxidase Deficiency Type II, Cortisol, Costello Syndrome, Cot Death, COVESDEM Syndrome, COX, COX Deficiency, COX Deficiency French-Canadian Type, COX Deficiency Infantile Mitochondrial myopathy de Toni-Fanconi-Debre included, COX Deficiency Type Benign Infantile Mitochondrial Myopathy, CP, CPEO, CPEO with myopathy, CPEO with Ragged-Red Fibers, CPPD Familial Form, CPT Deficiency, CPTD, Cranial Arteritis, Cranial Meningoencephalocele, Cranio-Oro-Digital Syndrome, Craniocarpotarsal dystrophy, Craniocele, Craniodigital Syndrome-Mental Retardation Scott Type, Craniofacial Dysostosis, Craniofacial Dysostosis-PD Arteriosus-Hypertrichosis-Hypoplasia of Labia, Craniofrontonasal Dysplasia, Craniometaphyseal Dysplasia, Cranioorodigital Syndrome, Cranioorodigital Syndrome Type II, Craniostenosis Crouzon Type, Craniostenosis, Craniosynostosis-Choanal Atresia-Radial Humeral Synostosis, Craniosynostosis-Hypertrichosis-Facial and Other Anomalies, Craniosynostosis Midfacial Hypoplasia and Foot Abnormalities, Craniosynostosis Primary, Craniosynostosis-Radial Aplasia Syndrome, Craniosynostosis with Radial Defects, Cranium Bifidum, CREST Syndrome, Creutzfeldt Jakob Disease, Cri du Chat Syndrome, Crib Death, Crigler Najjar Syndrome Type I, Crohn's Disease, Cronkhite-Canada Syndrome, Cross Syndrome, Cross' Syndrome, Cross-McKusick-Breen Syndrome, Crouzon, Crouzon Syndrome, Crouzon Craniofacial Dysostosis, Cryoglobulinemia Essential Mixed, Cryptophthalmos-Syndactyly Syndrome, Cryptorchidism-Dwarfism-Subnormal Mentality, Crystalline Corneal Dystrophy of Schnyder, CS, CSD, CSID, CSO, CST Syndrome, Curly Hair-Ankyloblephanon-Nail Dysplasia, Curschmann-Batten-Steinert Syndrome, Curth Macklin Type Ichthyosis Hystric, Curth-Macklin Type, Cushing's, Cushing Syndrome, Cushing's III, Cutaneous Malignant Melanoma Hereditary, Cutaneous Porphyrias, Cutis Laxa, Cutis Laxa-Growth Deficiency Syndrome, Cutis Marmorata Telangiectatica Congenita, CVI, CVID, CVS, Cyclic vomiting syndrome, Cystic Disease of the Renal Medulla, Cystic Hygroma, Cystic Fibrosis, Cystic Lymphangioma, Cystine-Lysine-Arginine-Ornithinuria, Cystine Storage Disease, Cystinosis, Cystinuria, Cystinuria with Dibasic Aminoaciduria, Cystinuria Type I, Cystinuria Type II, Cystinuria Type III, Cysts of the Renal Medulla Congenital, Cytochrome C Oxidase Deficiency, Dacryosialoadenopathy, Dacryosialoadenopathia, Dalpro, Dalton, Daltonism, Danbolt-Cross Syndrome, Dancing Eyes-Dancing Feet Syndrome, Dandy-Walker Syndrome, Dandy-Walker Cyst, Dandy-Walker Deformity, Dandy Walker Malformation, Danish Cardiac Type Amyloidosis (Type III), Darier Disease, Davidson's Disease, Davies' Disease, DBA, DBS, DC, DD, De Barsy Syndrome, De Barsy-Moens-Diercks Syndrome, de Lange Syndrome, De Morsier Syndrome, De Santis Cacchione Syndrome, de Toni-Fanconi Syndrome, Deafness Congenital and Functional Heart Disease, Deafness-Dwarfism-Retinal Atrophy, Deafness-Functional Heart Disease, Deafness Onychodystrophy Osteodystrophy and Mental Retardation, Deafness and Pili Torti Bjornstad Type, Deafness Sensorineural with Imperforate Anus and Hypoplastic Thumbs, Debrancher Deficiency, Deciduous Skin, Defect of Enterocyte Intrinsic Factor Receptor, Defect in Natural Killer Lymphocytes, Defect of Renal Reabsorption of Carnitine, Deficiency of Glycoprotein Neuraminidase, Deficiency of Mitochondrial Respiratory Chain Complex IV, Deficiency of Platelet Glycoprotein Ib, Deficiency of Von Willebrand Factor Receptor, Deficiency of Short-Chain Acyl-CoA Dehydrogenase (ACADS), Deformity with Mesomelic Dwarfism, Degenerative Chorea, Degenerative Lumbar Spinal Stenosis, Degos Disease, Degos-Kohlmeier Disease, Degos Syndrome, DEH, Dejerine-Roussy Syndrome, Dejerine Sottas Disease, Deletion 9p Syndrome Partial, Deletion 11q Syndrome Partial, Deletion 13q Syndrome Partial, Delleman-Oorthuys Syndrome, Delleman Syndrome, Dementia with Lobar Atrophy and Neuronal Cytoplasmic Inclusions, Demyelinating Disease, DeMyer Syndrome, Dentin Dysplasia Coronal, Dentin Dysplasia Radicular, Dentin Dysplasia Type I, Dentin Dysplasia Type II, Dentinogenesis Imperfecta Brandywine type, Dentinogenesis Imperfecta Shields Type, Dentinogenesis Imperfecta Type III, Dento-Oculo-Osseous Dysplasia, Dentooculocutaneous Syndrome, Denys-Drash Syndrome, Depakene, Depakene™ exposure, Depakote, Depakote Sprinkle, Depigmentation-Gingival Fibromatosis-Microphthalmia, Dercum Disease, Dermatitis Atopic, Dermatitis Exfoliativa, Dermatitis Herpetiformis, Dermatitis Multiformis, Dermatochalasia Generalized, Dermatolysis Generalized, Dermatomegaly, Dermatomyositis sine myositis, Dermatomyositis, Dermatosparaxis, Dermatostomatitis Stevens Johnson Type, Desbuquois Syndrome, Desmin Storage myopathy, Desquamation of Newborn, Deuteranomaly, Developmental Reading Disorder, Developmental Gerstmann Syndrome, Devergie Disease, Devic Disease, Devic Syndrome, Dextrocardia-Bronchiectasis and Sinusitis, Dextrocardia with Situs Inversus, DGS, DGSX Golabi-Rosen Syndrome Included, DH, DHAP alkyl transferase deficiency, DHBS Deficiency, DHOF, DHPR Deficiency, Diabetes Insipidus, Diabetes Insipidus Diabetes Mellitus Optic Atrophy and Deafness, Diabetes Insipidus Neurohypophyseal, Diabetes Insulin Dependent, Diabetes Mellitus, Diabetes Mellitus Addison's Disease Myxedema, Diabetic Acidosis, Diabetic Bearded Woman Syndrome, Diamond-Blackfan Anemia, Diaphragmatic Apnea, Diaphyseal Aclasis, Diastrophic Dwarfism, Diastrophic Dysplasia, Diastrophic Nanism Syndrome, Dicarboxylic Aminoaciduria, Dicarboxylicaciduria Caused by Defect in β-Oxidation of Fatty Acids, Dicarboxylicaciduria due to Defect in β-Oxidation of Fatty Acids, Dicarboxylicaciduria due to MCADH Deficiency, Dichromasy, Dicker-Opitz, DIDMOAD, Diencephalic Syndrome, Diencephalic Syndrome of Childhood, Diencephalic Syndrome of Emaciation, Dienoyl-CoA Reductase Deficiency, Diffuse Cerebral Degeneration in Infancy, Diffuse Degenerative Cerebral Disease, Diffuse Idiopathic Skeletal Hyperostosis, Diffusum-Glycopeptiduria, DiGeorge Syndrome, Digital-Oro-Cranio Syndrome, Digito-Oto-Palatal Syndrome, Digito-Oto-Palatal Syndrome Type I, Digito-Oto-Palatal Syndrome Type II, Dihydrobiopterin Synthetase Deficiency, Dihydropteridine Reductase Deficiency, Dihydroxyacetonephosphate synthase, Dilated (Congestive) Cardiomyopathy, Dimitri Disease, Diplegia of Cerebral Palsy, Diplo-Y Syndrome, Disaccharidase Deficiency, Disaccharide Intolerance I, Discoid Lupus, Discoid Lupus Erythematosus, DISH, Disorder of Cornification, Disorder of Cornification Type I, Disorder of Cornification 4, Disorder of Cornification 6, Disorder of Cornification 8, Disorder of Cornification 9 Netherton's Type, Disorder of Cornification 11 Phytanic Acid Type, Disorder of Cornification 12 (Neutral Lipid Storage Type); Disorder of Conification 13, Disorder of Cornification 14, Disorder of Cornification 14 Trichothiodystrophy Type, Disorder of Cornification 15 (Keratitis Deafness Type), Disorder of Cornification 16, Disorder of Cornification 18 Erythrokeratodermia Variabilis Type, Disorder of Cornification 19, Disorder of Cornification 20, Disorder of Cornification 24, Displaced Spleen, Disseminated Lupus Erythematosus, Disseminated Neurodermatitis, Disseminated Sclerosis, Distal 11q Monosomy, Distal 11q-Syndrome, Distal Arthrogryposis Multiplex Congenita Type IIA, Distal Arthrogryposis Multiplex Congenita Type IIA, Distal Arthrogryposis Type IIA, Distal Arthrogryposis Type 2A, Distal Duplication 6q, Distal Duplication 10q, Dup(10q) Syndrome, Distal Duplication 15q, Distal Monosomy 9p, Distal Trisomy 6q, Distal Trisomy 10q Syndrome, Distal Trisomy 11q, Divalproex, DJS, DKC, DLE, DLPIII, DM, DMC Syndrome, DMC Disease, DMD, DNS Hereditary, DOC 11, DOC 2, DOC 4, DOC 6 (Harlequin Type), DOC 8 Curth-Macklin Type, DOC 11 Phytanic Acid Type, DOC 12 (Neutral Lipid Storage Type), DOC 13, DOC 14, DOC 14 Trichothiodystrophy Type, DOC 15 (Keratitis Deafness Type), DOC 16, DOC 16 Unilateral Hemidysplasia Type, DOC 18, DOC 19, DOC 20, DOC 24, Dohle's Bodies-Myelopathy, Dolichospondylic Dysplasia, Dolichostenomelia, Dolichostenomelia Syndrome, Dominant Type Kenny-Caffe Syndrome, Dominant Type Myotonia Congenita, Donahue Syndrome, Donath-Landsteiner Hemolytic Anemia, Donath-Landsteiner Syndrome, DOOR Syndrome, DOORS Syndrome, Dopa-responsive Dystonia (DRD), Dorfman Chanarin Syndrome, Dowling-Meara Syndrome, Down Syndrome, DR Syndrome, Drash Syndrome, DRD, Dreifuss-Emery Type Muscular Dystrophy with Contractures, Dressler Syndrome, Drifting Spleen, Drug-induced Acanthosis Nigricans, Drug-induced Lupus Erythematosus, Drug-related Adrenal Insufficiency, Drummond's Syndrome, Dry Beriberi, Dry Eye, DTD, Duane's Retraction Syndrome, Duane Syndrome, Duane Syndrome Type IA 1B and 1C, Duane Syndrome Type 2A 2B and 2C, Duane Syndrome Type 3A 3B and 3C, Dubin Johnson Syndrome, Dubowitz Syndrome, Duchenne, Duchenne Muscular Dystrophy, Duchenne's Paralysis, Duhring's Disease, Duncan Disease, Duncan's Disease, Duodenal Atresia, Duodenal Stenosis, Duodenitis, Duplication 4p Syndrome, Duplication 6q Partial, Dupuy's Syndrome, Dupuytren's Contracture, Dutch-Kennedy Syndrome, Dwarfism, Dwarfism Campomelic, Dwarfism Cortical Thickening of the Tubular Bones & Transient Hypocalcemia, Dwarfism Levi's Type, Dwarfism Metatropic, Dwarfism-Onychodysplasia, Dwarfism-Pericarditis, Dwarfism with Renal Atrophy and Deafness, Dwarfism with Rickets, DWM, Dyggve Melchior Clausen Syndrome, Dysautonomia Familial, Dysbetalipoproteinemia Familial, Dyschondrodysplasia with Hemangiomas, Dyschondrosteosis, Dyschromatosis Universalis Hereditaria, Dysencephalia Splanchnocystica, Dyskeratosis Congenita, Dyskeratosis Congenita Autosomal Recessive, Dyskeratosis Congenita Scoggins Type, Dyskeratosis Congenita Syndrome, Dyskeratosis Follicularis Vegetans, Dyslexia, Dysmyelogenic Leukodystrophy, Dysmyelogenic Leukodystrophy-Megalobare, Dysphonia Spastica, Dysplasia Epiphysialis Punctata, Dysplasia Epiphyseal Hemimelica, Dysplasia of Nails With Hypodontia, Dysplasia Cleidocranial, Dysplasia Fibrous, Dysplasia Gigantism SyndromeX-Linked, Dysplasia Osteodental, Dysplastic Nevus Syndrome, Dysplastic Nevus Type, Dyssynergia Cerebellaris Myoclonica, Dyssynergia Esophagus, Dystonia, Dystopia Canthorum, Dystrophia Adiposogenitalis, Dystrophia Endothelialis Cornea, Dystrophia Mesodermalis, Dystrophic Epidermolysis Bullosa, Dystrophy, Asphyxiating Thoracic, Dystrophy Myotonic, E-D Syndrome, Eagle-Barrett Syndrome, Eales Retinopathy, Eales Disease, Ear Anomalies-Contractures-Dysplasia of Bone with Kyphoscoliosis, Ear Patella Short Stature Syndrome, Early Constraint Defects, Early Hypercalcemia Syndrome with Elfin Facie, Early-onset Dystonia, Eaton Lambert Syndrome, EB, Ebstein's anomaly, EBV Susceptibility (EBVS), EBVS, ECD, ECPSG, Ectodermal Dysplasias, Ectodermal Dysplasia Anhidrotic with Cleft Lip and Cleft Palate, Ectodermal Dysplasia-Exocrine Pancreatic Insufficiency, Ectodermal Dysplasia Rapp-Hodgkin type, Ectodermal and Mesodermal Dysplasia Congenital, Ectodermal and Mesodermal Dysplasia with Osseous Involvement, Ectodermosis Erosiva Pluriorificialis, Ectopia Lentis, Ectopia Vesicae, Ectopic ACTH Syndrome, Ectopic Adrenocorticotropic Hormone Syndrome, Ectopic Anus, Ectrodactilia of the Hand, Ectrodactyly, Ectrodactyly-Ectodermal Dysplasia-Clefting Syndrome, Ectrodactyly Ectodermal Dysplasias Clefting Syndrome, Ectrodactyly Ectodermal Dysplasia Cleft Lip/Cleft Palate, Eczema, Eczema-Thrombocytopenia-Immunodeficiency Syndrome, EDA, EDMD, EDS, EDS Arterial-Ecchymotic Type, EDS Arthrochalasia, EDS Classic Severe Form, EDS Dysfibronectinemic, EDS Gravis Type, EDS Hypermobility, EDS Kyphoscoliotic, EDS Kyphoscoliosis, EDS Mitis Type, EDS Ocular-Scoliotic, EDS Progeroid, EDS Periodontosis, EDS Vascular, EEC Syndrome, EFE, EHBA, EHK, Ehlers Danlos Syndrome, Ehlers-Danlos syndrome, Ehlers Danlos IX, Eisenmenger Complex, Eisenmenger's complex, Eisenmenger Disease, Eisenmenger Reaction, Eisenmenger Syndrome, Ekbom Syndrome, Ekman-Lobstein Disease, Ektrodactyly of the Hand, EKV, Elastin fiber disorders, Elastorrhexis Generalized, Elastosis Dystrophica Syndrome, Elective Mutism (obsolete), Elective Mutism, Electrocardiogram (ECG or EKG), Electron Transfer Flavoprotein (ETF) Dehydrogenase Deficiency: (GAII & MADD), Electrophysiologic study (EPS), Elephant Nails From Birth, Elephantiasis Congenita Angiomatosa, Hemangiectatic Hypertrophy, Elfin Facies with Hypercalcemia, Ellis-van Creveld Syndrome, Ellis Van Creveld Syndrome, Embryoma Kidney, Embryonal Adenomyosarcoma Kidney, Embryonal Carcinosarcoma Kidney, Embryonal Mixed Tumor Kidney, EMC, Emery Dreyfus Muscular Dystrophy, Emery-Dreifuss Muscular Dystrophy, Emery-Dreifuss Syndrome, EMF, EMG Syndrome, Empty Sella Syndrome, Encephalitis Periaxialis Diffusa, Encephalitis Periaxialis Concentrica, Encephalocele, Encephalofacial Angiomatosis, Encephalopathy, Encephalotrigeminal Angiomatosis, Enchondromatosis with Multiple Cavernous Hemangiomas, Endemic Polyneuritis, Endocardial Cushion Defect, Endocardial Cushion Defects, Endocardial Dysplasia, Endocardial Fibroelastosis (EFE), Endogenous Hypertriglyceridemia, Endolymphatic Hydrops, Endometrial Growths, Endometriosis, Endomyocardial Fibrosis, Endothelial Corneal Dystrophy Congenital, Endothelial Epithelial Corneal Dystrophy, Endothelium, Engelmann Disease, Enlarged Tongue, Enterocolitis, Enterocyte Cobalamin Malabsorption, Eosinophia Syndrome, Eosinophilic Cellulitis, Eosinophilic Fasciitis, Eosinophilic Granuloma, Eosinophilic Syndrome, Epidermal Nevus Syndrome, Epidermolysis Bullosa, Epidermolysis Bullosa Acquisita, Epidermolysis Bullosa Hereditaria, Epidermolysis Bullosa Letalias, Epidermolysis Hereditaria Tarda, Epidermolytic Hyperkeratosis, Epidermolytic Hyperkeratosis (Bullous CIE), Epilepsia Procursiva, Epilepsy, Epinephrine, Epiphyseal Changes and High Myopia, Epiphyseal Osteochondroma Benign, Epiphysealis Hemimelica Dysplasia, Episodic-Abnormal Eye Movement, Epithelial Basement Membrane Corneal Dystrophy, Epithelial Corneal Dystrophy of Meesmann Juvenile, Epitheliomatosis Multiplex with Nevus, Epithelium, Epival, EPS, Epstein-Barr Virus-Induced Lymphoproliferative Disease in Males, Erb-Goldflam syndrome, Erdheim Chester Disease, Erythema Multiforme Exudativum, Erythema Polymorphe Stevens Johnson Type, Erythroblastophthisis, Erythroblastosis Fetalis, Erythroblastosis Neonatorum, Erythroblastotic Anemia of Childhood, Erythrocyte Phosphoglycerate Kinase Deficiency, Erythrogenesis Imperfecta, Erythrokeratodermia Progressiva Symmetrica, Erythrokeratodermia Progressiva Symmetrica Ichlithyosis, Erythrokeratodermia Variabilis, Erythrokeratodermia Variabilis Type, Erythrokeratolysis Hiemalis, Erythropoietic Porphyrias, Erythropoietic Porphyria, Escobar Syndrome, Esophageal Atresia, Esophageal Aperistalsis, Esophagitis-Peptic Ulcer, Esophagus Atresia and/or Tracheoesophageal Fistula, Essential Familial Hyperlipemia, Essential Fructosuria, Essential Hematuria, Essential Hemorrhagic Thrombocythemia, Essential Mixed Cryoglobulinemia, Essential Moschowitz Disease, Essential Thrombocythemia. Essential Thrombocytopenia, Essential Thrombocytosis, Essential Tremor, Esterase Inhibitor Deficiency, Estren-Dameshek variant of Fanconi Anemia, Estrogen-related Cholestasis, ET, ETF, Ethylmalonic Adipicaciduria, Eulenburg Disease, pc, EVCS, Exaggerated Startle Reaction, Exencephaly, Exogenous Hypertriglyceridemia, Exomphalos-Macroglossia-Gigantism Syndrome, Exophthalmic Goiter, Expanded Rubella Syndrome, Exstrophy of the Bladder, EXT, External Chondromatosis Syndrome, Extrahepatic Biliary Atresia, Extramedullary Plasmacytoma, Exudative Retinitis, Eye Retraction Syndrome, FA1, FAA, Fabry Disease, FAC, FACB, FACD, FACE, FACF, FACG, FACH, Facial Nerve Palsy, Facial Paralysis, Facial Ectodermal Dysplasias, Facial Ectodermal Dysplasia, Facio-Scapulo-Humeral Dystrophy, Facio-Auriculo-Vertebral Spectrum, Facio-cardio-cutaneous syndrome, Facio-Fronto-Nasal Dysplasia, Faciocutaneoskeletal Syndrome, Faciodigitogenital syndrome, Faciogenital dysplasia, Faciogenitopopliteal Syndrome, Faciopalatoosseous Syndrome, Faciopalatoosseous Syndrome Type II, Facioscapulohumeral muscular dystrophy, Factitious Hypoglycemia, Factor VIII Deficiency, Factor IX Deficiency, Factor XI Deficiency, Factor XII deficiency, Factor XIII Deficiency, Fahr Disease, Fahr's Disease, Failure of Secretion Gastric Intrinsic Factor, Fairbank Disease, Fallot's Tetralogy, Familial Acrogeria, Familial Acromicria, Familial Adenomatous Colon Polyposis, Familial Adenomatous Polyposis with Extraintestinal Manifestations, Familial Alobar Holoprosencephaly, Familial Alpha-Lipoprotein Deficiency, Familial Amyotrophic Chorea with Acanthocytosis, Familial Arrhythmic Myoclonus, Familial Articular Chondrocalcinosis, Familial Atypical Mole-Malignant Melanoma Syndrome, Familial Broad Beta Disease, Familial Calcium Gout, Familial Calcium Pyrophosphate Arthropathy, Familial Chronic Obstructive Lung Disease, Familial Continuous Skin Peeling, Familial Cutaneous Amyloidosis, Familial Dysproteinemia, Familial Emphysema, Familial Enteropathy Microvillus, Familial Foveal Retinoschisis, Familial Hibernation Syndrome, Familial High Cholesterol, Familial Hemochromatosis, Familial High Blood Cholesterol, Familial High-Density Lipoprotein Deficiency, Familial High Serum Cholesterol, Familial Hyperlipidema, Familial Hypoproteinemia with Lymphangietatic Enteropathy, Familial Jaundice, Familial Juvenile Nephronophtisis-Associated Ocular Anomaly, Familial Lichen Amyloidosis (Type IX), Familial Lumbar Stenosis, Familial Lymphedema Praecox, Familial Mediterranean Fever, Familial Multiple Polyposis, Familial Nuchal Bleb, Familial Paroxysmal Polyserositis, Familial Polyposis Coli, Familial Primary Pulmonary Hypertension, Familial Renal Glycosuria, Familial Splenic Anemia, Familial Startle Disease, Familial Visceral Amyloidosis (Type VIII), FAMMM, FANCA, FANCB, FANCC, FANCD, FANCE, Fanconi Panmyelopathy, Fanconi Pancytopenia, Fanconi II, Fanconi's Anemia, Fanconi's Anemia Type I, Fanconi's Anemia Complementation Group, Fanconi's Anemia Complementation Group A, Fanconi's Anemia Complementation Group B, Fanconi's Anemia Complementation Group C, Fanconi's Anemia Complementation Group D, Fanconi's Anemia Complementation Group E, Fanconi's Anemia Complementation Group G, Fanconi's Anemia Complementation Group H, Fanconi's Anemia Estren-Dameshek Variant, FANF, FANG, FANH, FAP, FAPG, Farber's Disease, Farber's Lipogranulomatosis, FAS, Fasting Hypoglycemia, Fat-Induced Hyperlipemia, Fatal Granulomatous Disease of Childhood, Fatty Oxidation Disorders, Fatty Liver with Encephalopathy, FAV, FCH, FCMD, FCS Syndrome, FD, FDH, Febrile Mucocutaneous Syndrome Stevens Johnson Type, Febrile Neutrophilic Dermatosis Acute, Febrile Seizures, Feinberg's syndrome, Feissinger-Leroy-Reiter Syndrome, Female Pseudo-Turner Syndrome, Femoral Dysgenesis Bilateral-Robin Anomaly, Femoral Dysgenesis Bilateral, Femoral Facial Syndrome, Femoral Hypoplasia-Unusual Facies Syndrome, Fetal Alcohol Syndrome, Fetal Anti-Convulsant Syndrome, Fetal Cystic Hygroma, Fetal Effects of Alcohol, Fetal Effects of Chickenpox, Fetal Effects of Thalidomide, Fetal Effects of Varicella Zoster Virus, Fetal Endomyocardial Fibrosis, Fetal Face Syndrome, Fetal Iritis Syndrome, Fetal Transfusion Syndrome, Fetal Valproate Syndrome, Fetal Valproic Acid Exposure Syndrome, Fetal Varicella Infection, Fetal Varicella Zoster Syndrome, FFDD Type II, FG Syndrome, FGDY, FHS, Fibrin Stabilizing Factor Deficiency, Fibrinase Deficiency, Fibrinoid Degeneration of Astrocytes, Fibrinoid Leukodystrophy, Fibrinoligase Deficiency, Fibroblastoma Perineural, Fibrocystic Disease of Pancreas, Fibrodysplasia Ossificans Progressiva, Fibroelastic Endocarditis, Fibromyalgia, Fibromyalgia-Fibromyositis, Fibromyositis, Fibrosing Cholangitis, Fibrositis, Fibrous Ankylosis of Multiple Joints, Fibrous Cavernositis, Fibrous Dysplasia, Fibrous Plaques of the Penis, Fibrous Sclerosis of the Penis, Fickler-Winkler Type, Fiedler Disease, Fifth Digit Syndrome, Filippi Syndrome, Finnish Type Amyloidosis (Type V), First Degree Congenital Heart Block, First and Second Branchial Arch Syndrome, Fischer's Syndrome, Fish Odor Syndrome, Fissured Tongue, Flat Adenoma Syndrome, Flatau-Schilder Disease, Flavin Containing Monooxygenase 2, Floating β Disease, Floating-Harbor Syndrome, Floating Spleen, Floppy Infant Syndrome, Floppy Valve Syndrome, Fluent Aphasia, FMD, FMF, FMO Adult Liver Form, FMO2, FND, Focal Dermal Dysplasia Syndrome, Focal Dermal Hypoplasia, Focal Dermato-Phalangeal Dysplasia, Focal Dystonia, Focal Epilepsy, Focal Facial Dermal Dysplasia Type II, Focal Neuromyotonia, FODH, Folling Syndrome, Fong Disease, FOP, Forbes Disease, Forbes-Albright Syndrome, Forestier's Disease, Forsius-Eriksson Syndrome (X-Linked), Fothergill Disease, Fountain Syndrome, Foveal Dystrophy Progressive, FPO Syndrome Type II, FPO, Fraccaro Type Achondrogenesis (Type IB), Fragile X syndrome, Franceschetti-Zwalen-Klein Syndrome, Francois Dyscephaly Syndrome, Francois-Neetens Speckled Dystrophy, Flecked Corneal Dystrophy, Fraser Syndrome, FRAXA, FRDA, Fredrickson Type I Hyperlipoproteinemia, Freeman-Sheldon Syndrome, Freire-Maia Syndrome, Frey's Syndrome, Friedreich's Ataxia, Friedreich's Disease, Friedreich's Tabes, FRNS, Froelich's Syndrome, Frommel-Chiari Syndrome, Frommel-Chiari Syndrome Lactation-Uterus Atrophy, Frontodigital Syndrome, Frontofacionasal Dysostosis, Frontofacionasal Dysplasia, Frontonasal Dysplasia, Frontonasal Dysplasia with Coronal Craniosynostosis, Fructose-1-Phosphate Aldolase Deficiency, Fructosemia, Fructosuria, Fryns Syndrome, FSH, FSHD, FSS, Fuchs Dystrophy, Fucosidosis Type 1, Fucosidosis Type 2, Fucosidosis Type 3, Fukuhara Syndrome, Fukuyama Disease, Fukuyama Type Muscular Dystrophy, Fumarylacetoacetase Deficiency, Furrowed Tongue, G Syndrome, G6PD Deficiency, G6PD, GA I, GA IIB, GA IIA, GA II, GAII & MADD, Galactorrhea-Amenorrhea Syndrome Nonpuerperal, Galactorrhea-Amenorrhea without Pregnancy, Galactosamine-6-Sulfatase Deficiency, Galactose-1-Phosphate Uridyl Transferase Deficiency, Galactosemia, GALB Deficiency, Galloway-Mowat Syndrome, Galloway Syndrome, GALT Deficiency, Gammaglobulin Deficiency, GAN, Ganglioside Neuraminidase Deficiency, Ganglioside Sialidase Deficiency, Gangliosidosis GM1 Type 1, Gangliosidosis GM2 Type 2, Gangliosidosis β Hexosaminidase B Deficiency, Gardner Syndrome, Gargoylism, Garies-Mason Syndrome, Gasser Syndrome, Gastric Intrinsic Factor Failure of Secretion, Enterocyte Cobalamin, Gastrinoma, Gastritis, Gastroesophageal Laceration-Hemorrhage, Gastrointestinal Polyposis and Ectodermal Changes, Gastroschisis, Gaucher Disease, Gaucher-Schlagenhaufer, Gayet-Wernicke Syndrome, GBS, GCA, GCM Syndrome, GCPS, Gee-Herter Disease, Gee-Thaysen Disease, Gehrig's Disease, Gelineau's Syndrome, Genee-Wiedemann Syndrome, Generalized Dystonia, Generalized Familial Neuromyotonia, Generalized Fibromatosis, Generalized Flexion Epilepsy, Generalized Glycogenosis, Generalized Hyperhidrosis, Generalized Lipofuscinosis, Generalized Myasthenia Gravis, Generalized Myotonia, Generalized Sporadic Neuromytonia, Genetic Disorders, Genital Defects, Genital and Urinary Tract Defects, Gerstmann Syndrome, Gerstmann Tetrad, GHBP, GHD, GHR, Giant Axonal Disease, Giant Axonal Neuropathy, Giant Benign Lymphoma, Giant Cell Glioblastoma Astrocytoma, Giant Cell Arteritis, Giant Cell Disease of the Liver, Giant Cell Hepatitis, Giant Cell of Newborns Cirrhosis, Giant Cyst of the Retina, Giant Lymph Node Hyperplasia, Giant Platelet Syndrome Hereditary, Giant Tongue, Macular Dystrophy, Gilbert's Disease, Gilbert Syndrome, Gilbert-Dreyfus Syndrome, Gilbert-Lereboullet Syndrome, Gilford Syndrome, Gilles de la Tourette's syndrome, Gillespie Syndrome, Gingival Fibromatosis-Abnormal Fingers Nails Nose Ear Splenomegaly, GLA Deficiency, GLA, GLB1, Glioma Retina, Global Aphasia, Globoid Leukodystrophy, Glossoptosis Micrognathia and Cleft Palate, Glucocerebrosidase Deficiency, Glucocerebrosidosis, Glucose-6-Phosphate Dehydrogenase Deficiency, Glucose-6-Phosphate Tranport Defect, Glucose-6-Phospate Translocase Deficiency, Glucose-G-Phosphatase Deficiency, Glucose-Galactose Malabsorption, Glucosyl Ceramide Lipidosis, Glutaric Aciduria I, Glutaric Acidemia I, Glutaric Acidemia II, Glutaric Aciduria II, Glutaric Aciduria Type II, Glutaric Aciduria Type III, Glutaricacidemia I, Glutaricacidemia II, Glutaricaciduria I, Glutaricaciduria II, Glutaricaciduria Type IIA, Glutaricaciduria Type IIB, Glutaryl-CoA Dehydrogenase Deficiency, Glutaurate-Aspartate Transport Defect, Gluten-Sensitive Enteropathy, Glycogen Disease of Muscle Type VII, Glycogen Storage Disease I, Glycogen Storage Disease III, Glycogen Storage Disease IV, Glycogen Storage Disease Type V, Glycogen Storage Disease VI, Glycogen Storage Disease VII, Glycogen Storage Disease VIII, Glycogen Storage Disease Type II, Glycogen Storage Disease-Type II, Glycogenosis, Glycogenosis Type I, Glycogenosis Type IA, Glycogenosis Type IB, Glycogenosis Type II, Glycogenosis Type II, Glycogenosis Type III, Glycogenosis Type IV, Glycogenosis Type V, Glycogenosis Type VI, Glycogenosis Type VII, Glycogenosis Type VIII, Glycolic Aciduria, Glycolipid Lipidosis, GM2 Gangliosidosis Type 1, GM2 Gangliosidosis Type 1, GNPTA, Goitrous Autoimmune Thyroiditis, Goldenhar Syndrome, Goldenhar-Gorlin Syndrome, Goldscheider's Disease, Goltz Syndrome, Goltz-Gorlin Syndrome, Gonadal Dysgenesis 45 X, Gonadal Dysgenesis XO, Goniodysgenesis-Hypodontia, Goodman Syndrome, Goodman, Goodpasture Syndrome, Gordon Syndrome, Gorlin's Syndrome, Gorlin-Chaudhry-Moss Syndrome, Gottron Erythrokeratodermia Congenitalis Progressiva Symmetrica, Gottron's Syndrome, Gougerot-Carteaud Syndrome, Graft-versus Host Disease, Grand Mal Epilepsy, Granular Type Corneal Dystrophy, Granulomatous Arteritis, Granulomatous Colitis, Granulomatous Dermatitis with Eosinophilia, Granulomatous Ileitis, Graves Disease, Graves' Hyperthyroidism, Graves' Disease, Greig Cephalopolysyndactyly Syndrome, Groenouw Type I Corneal Dystrophy, Groenouw Type II Corneal Dystrophy, Gronblad-Strandberg Syndrome, Grotton Syndrome, Growth Hormone Receptor Deficiency, Growth Hormone Binding Protein Deficiency, Growth Hormone Deficiency, Growth-Mental Deficiency Syndrome of Myhre, Growth Retardation-Rieger Anomaly, GRS, Gruber Syndrome, GS, GSD6, GSD8, GTS, Guanosine Triphosphate-Cyclohydrolase Deficiency, Guanosine Triphosphate-Cyclohydrolase Deficiency, Guenther Porphyria, Guerin-Stern Syndrome, Guillain-Barré, Guillain-BarréSyndrome, Gunther Disease, H Disease, H. Gottron's Syndrome, Habit Spasms, HAE, Hageman Factor Deficiency, Hageman factor, Haim-Munk Syndrome, Hajdu-Cheney Syndrome, Hajdu Cheney, HAL Deficiency, Hall-Pallister Syndrome, Hallermann-Streiff-Francois Syndrome, Hallernann-Streiff Syndrome, Hallervorden-Spatz Disease, Hallervorden-Spatz Syndrome, Hallopeau-Siemens Disease, Hallux Duplication Postaxial Polydactyly and Absence of Corpus Callosum, Halushi-Behcet's Syndrome, Hamartoma of the Lymphatics, Hand-Schueller-Christian Syndrome, HANE, Hanhart Syndrome, Happy Puppet Syndrome, Harada Syndrome, HARD+/−E Syndrome, HARD Syndrome, Hare Lip, Harlequin Fetus, Harlequin Type DOC 6, Harlequin Type Ichthyosis, Harley Syndrome, Harrington Syndrome, Hart Syndrome, Hartnup Disease, Hartnup Disorder, Hartnup Syndrome, Hashimoto's Disease, Hashimoto-Pritzker Syndrome, Hashimoto's Syndrome, Hashimoto's Thyroiditis, Hashimoto-Pritzker Syndrome, Hay Well's Syndrome, Hay-Wells Syndrome of Ectodermal Dysplasia, HCMM, HCP, HCTD, HD, Heart-Hand Syndrome (Holt-Oram Type), Heart Disease, Hecht Syndrome, HED, Heerferdt-Waldenstrom and Lofgren's Syndromes, Hegglin's Disease, Heinrichsbauer Syndrome, Hemangiomas, Hemangioma Familial, Hemangioma-Thrombocytopenia Syndrome, Hemangiomatosis Chondrodystrophica, Hemangiomatous Branchial Clefts-Lip Pseudocleft Syndrome, Hemifacial Microsomia, Hemimegalencephaly, Hemiparesis of Cerebral Palsy, Hemiplegia of Cerebral Palsy, Hemisection of the Spinal Cord, Hemochromatosis, Hemochromatosis Syndrome, Hemodialysis-Related Amyloidosis, Hemoglobin Lepore Syndromes, Hemolytic Anemia of Newborn, Hemolytic Cold Antibody Anemia, Hemolytic Disease of Newborn, Hemolytic-Uremic Syndrome, Hemophilia, Hemophilia A, Hemophilia B, Hemophilia B Factor IX, Hemophilia C, Hemorrhagic Dystrophic Thrombocytopenia, Hemorrhagica Aleukia, Hemosiderosis, Hepatic Fructokinase Deficiency, Hepatic Phosphorylase Kinase Deficiency, Hepatic Porphyria, Hepatic Porphyrias, Hepatic Veno-Occlusive Disease, Hepato-Renal Syndrome, Hepatolenticular Degeneration, Hepatophosphorylase Deficiency, Hepatorenal Glycogenosis, Hepatorenal Syndrome, Hepatorenal Tyrosinemia, Hereditary Acromelalgia, Hereditary Alkaptonuria, Hereditary Amyloidosis, Hereditary Angioedema, Hereditary Areflexic Dystasia, Heredopathia Atactica Polyneuritiformis, Hereditary Ataxia, Hereditary Ataxia Friedrich's Type, Hereditary Benign Acanthosis Nigricans, Hereditary Cerebellar Ataxia, Hereditary Chorea, Hereditary Chronic Progressive Chorea, Hereditary Connective Tissue Disorders, Hereditary Coproporphyria, Hereditary Coproporphyria Porphyria, Hereditary Cutaneous Malignant Melanoma, Hereditary Deafness-Retinitis Pigmentosa, Heritable Disorder of Zinc Deficiency, Hereditary DNS, Hereditary Dystopic Lipidosis, Hereditary Emphysema, Hereditary Fructose Intolerance, Hereditary Hemorrhagic Telangiectasia, Hereditary Hemorrhagic Telangiectasia Type I, Hereditary Hemorrhagic Telangiectasia Type III, Hereditary Hyperuricemia and Choreoathetdsis Syndrome, Hereditary Leptocytosis Major, Hereditary Leptocytosis Minor, Hereditary Lymphedema, Hereditary Lymphedema Tarda, Hereditary Lymphedema Type I, Hereditary Lymphedema Type II, Hereditary Motor Sensory Neuropathy, Hereditary Motor Sensory Neuropathy I, Hereditary Motor Sensory Neuropathy Type III, Hereditary Nephritis, Hereditary Nephritis and Nerve Deafness, Hereditary Nephropathic Amyloidosis, Hereditary Nephropathy and Deafness, Hereditary Nonpolyposis Colorectal Cancer, Hereditary Nonpolyposis Colorectal Carcinoma, Hereditary Nonspherocytic Hemolytic Anemia, Hereditary Onychoosteodysplasia, Hereditary Optic Neuroretinopathy, Hereditary Polyposis Coli, Hereditary Sensory and Autonomic Neuropathy Type I, Hereditary Sensory and Autonomic Neuropathy Type II, Hereditary Sensory and Autonomic Neuropathy Type III, Hereditary Sensory Motor Neuropathy, Hereditary Sensory Neuropathy Type I, Hereditary Sensory Neuropathy Type I, Hereditary Sensory Neuropathy Type II, Hereditary Sensory Neuropathy Type III, Hereditary Sensory Radicular Neuropathy Type I, Hereditary Sensory Radicular Neuropathy Type I, Hereditary Sensory Radicular Neuropathy Type II, Hereditary Site Specific Cancer, Hereditary Spherocytic Hemolytic Anemia, Hereditary Spherocytosis, Hereditary Tyrosinemia Type I, Heritable Connective Tissue Disorders, Herlitz Syndrome, Hermians-Herzberg Phakomatosis, Hermansky-Pudlak Syndrome, Hermaphroditism, Herpes Zoster, Herpes Iris Stevens-Johnson Type, Hers Disease, Heterozygous β Thalassemia, Hexoaminidase α-Subunit Deficiency (Variant B), Hexoaminidase α-Subunit Deficiency (Variant B), HFA, HFM, HOPS, HH, HHHO, HHRH, HHT, Hiatal Hernia-Microcephaly-Nephrosis Galloway Type, Hidradenitis Suppurativa, Hidrosadenitis Axillaris, Hidrosadenitis Suppurativa, Hidrotic Ectodermal Dysplasias, HIE Syndrome, High Imperforate Anus, High Potassium, High Scapula, HIM, Hirschsprung's Disease, Hirschsprung's Disease Acquired, Hirschsprung Disease Polydactyly of Ulnar & Big Toe and VSD, Hirschsprung Disease with Type D Brachydactyly, Hirsutism, HIS Deficiency, Histidine Ammonia-Lyase (HAL) Deficiency, Histidase Deficiency, Histidinemia, Histiocytosis, Histiocytosis X, HLHS, HLP Type II, HMG, HMI, HMSN I, HNHA, HOCM, Hodgkin Disease, Hodgkin's Disease, Hodgkin's Lymphoma, Hollaender-Simons Disease, Holmes-Adie Syndrome, Holocarboxylase Synthetase Deficiency, Holoprosencephaly, Holoprosencephaly Malformation Complex, Holoprosencephaly Sequence, Holt-Oram Syndrome, Holt-Oram Type Heart-Hand Syndrome, Homocystinemia, Homocystinuria, Homogentisic Acid Oxidase Deficiency, Homogentisic Acidura, Homozygous α-1-Antitrypsin Deficiency, HOOD, Horner Syndrome, Horton's, disease, HOS, HOS1, Houston-Harris Type Achrondrogenesis (Type IA), HPS, HRS, HS, HSAN Type I, HSAN Type II, HSAN-III, HSMN, HSMN Type III, HSN I, HSN-III, Huebner-Herter Disease, Hunner's Patch, Hunner's Ulcer, Hunter Syndrome, Hunter-Thompson Type Acromesomelic Dysplasia, Huntington's Chorea, Huntington's Disease, Hurler Disease, Hurler Syndrome, Hurler-Scheie Syndrome, HUS, Hutchinson-Gilford Progeria Syndrome, Hutchinson-Gilford Syndrome, Hutchinson-Weber-Peutz Syndrome, Hutterite Syndrome Bowen-Conradi Type, Hyaline Panneuropathy, Hydranencephaly, Hydrocephalus, Hydrocephalus Agyria and Retinal Dysplasia, Hydrocephalus Internal Dandy-Walker Type, Hydrocephalus Noncommunicating Dandy-Walker Type, Hydrocephaly, Hydronephrosis With Peculiar Facial Expression, Hydroxylase Deficiency, Hygroma Colli, Hyper-IgE Syndrome, Hyper-IgM Syndrome, Hyperaldosteronism, Hyperaldosteronism With Hypokalemic Alkatosis, Hyperaldosteronism Without Hypertension, Hyperammonemia, Hyperammonemia Due to Carbamylphosphate Synthetase Deficiency, Hyperammonemia Due to Ornithine Transcarbamylase Deficiency, Hyperammonemia Type II, Hyper-β Carnosinemia, Hyperbilirubinemia I, Hyperbilirubinemia II, Hypercalcemia Familial with Nephrocalcinosis and Indicanuria, Hypercalcemia-Supravalvar Aortic Stenosis, Hypercalciuric Rickets, Hypercapnic acidosis, Hypercatabolic Protein-Losing Enteropathy, Hyperchloremic acidosis, Hypercholesterolemia, Hypercholesterolemia Type IV, Hyperchylomicronemia, Hypercystinuria, Hyperekplexia, Hyperextensible joints, Hyperglobulinemic Purpura, Hyperglycinemia with Ketoacidosis and Lactic Acidosis Propionic Type, Hyperglycinemia Nonketotic, Hypergonadotropic Hypogonadism, Hyperimmunoglobulin E Syndrome, Hyperimmunoglobulin E-Recurrent Infection Syndrome, Hyperimmunoglobulinemia E-Staphylococcal, Hyperkalemia, Hyperkinetic Syndrome, Hyperlipemic Retinitis, Hyperlipidemia I, Hyperlipidemia IV, Hyperlipoproteinemia Type I, Hyperlipoproteinemia Type III, Hyperlipoproteinemia Type IV, Hyperoxaluria, Hyperphalangy-Clinodactyly of Index Finger with Pierre Robin Syndrome, Hyperphenylalanemia, Hyperplastic Epidernolysis Bullosa, Hyperpnea, Hyperpotassemia, Hyperprebeta-Lipoproteinemia, Hyperprolinemia Type I, Hyperprolinemia Type II, Hypersplenism, Hypertelorism with Esophageal Abnormalities and Hypospadias, Hypertelorism-Hypospadias Syndrome, Hypertrophic Cardio myopathy, Hypertrophic Interstitial Neuropathy, Hypertrophic Interstitial Neuritis, Hypertrophic Interstitial Radiculoneuropathy, Hypertrophic Neuropathy of Refsum, Hypertrophic Obstructive Cardio myopathy, Hyperuricemia Choreoathetosis Self-mutilation Syndrome, Hyperuricemia-Oligophrenia, Hypervalinemia, Hypocalcified (Hypomineralized) Type, Hypochondrogenesis, Hypochrondroplasia, Hypo-γ-globulinemia, Hypo-γ-globulinemia Transient of Infancy, Hypogenital Dystrophy with Diabetic Tendency, Hypoglossia-Hypodactylia Syndrome, Hypoglycemia, Exogenous Hypoglycemia, Hypoglycemia with Macroglossia, Hypoglycosylation Syndrome Type 1a, Hypoglycosylation Syndrome Type 1a, Hypogonadism with Anosmia, Hypogonadotropic Hypogonadism and Anosmia, Hypohidrotic Ectodermal Dysplasia, Hypohidrotic Ectodermal Dysplasia Autosomal Dominant type, Hypohidrotic Ectodermal Dysplasias Auto-recessive, Hypokalemia, Hypokalemic Alkalosis with Hypercalciuria, Hypokalemic Syndrome, Hypolactasia, Hypomaturation Type (Snow-Capped Teeth), Hypomelanosis of Ito, Hypomelia-Hypotrichosis-Facial Hemangioma Syndrome, Hypomyelination Neuropathy, Hypoparathyroidism, Hypophosphatasia, Hypophosphatemic Rickets with Hypercalcemia, Hypopigmentation, Hypopigmented macular lesion, Hypoplasia of the Depressor Anguli Oris Muscle with Cardiac Defects, Hypoplastic Anemia, Hypoplastic Congenital Anemia, Hypoplastic Chondrodystrophy, Hypoplastic Enamel-Onycholysis-Hypohidrosis, Hypoplastic (Hypoplastic-Explastic) Type, Hypoplastic Left Heart Syndrome, Hypoplastic-Triphalangeal Thumbs, Hypopotassemia Syndrome, Hypospadias-Dysphagia Syndrome, Hyposmia, Hypothalamic Hamartoblastoma Hypopituitarism Imperforate Anus Polydactyly, Hypothalamic Infantilism-Obesity, Hypothyroidism, Hypotonia-Hypomentia-Hypogonadism-Obesity Syndrome, Hypoxanthine-Guanine Phosphoribosyltransferase Defect (Complete Absence of), I-Cell Disease, Iatrogenic Hypoglycemia, IBGC, IBIDS Syndrome, IBM, IBS, IC, I-Cell Disease, ICD, ICE Syndrome Cogan-Reese Type, Icelandic Type Amyloidosis (Type VI), I-Cell Disease, Ichthyosiform Erythroderma Corneal Involvement and Deafness, Ichthyosiform Erythroderma Hair Abnormality Growth and Men, Ichthyosiform Erythroderma with Leukocyte Vacuolation, Ichthyosis, Ichthyosis Congenita, Ichthyosis Congenital with Trichothiodystrophy, Ichthyosis Hystrix, Ichthyosis Hystrix Gravior, Ichthyosis Linearis Circumflexa, Ichthyosis Simplex, Ichthyosis Tay Syndrome, Ichthyosis Vulgaris, Ichthyotic Neutral Lipid Storage Disease, Icteric Leptospirosis, Icterohemorrhagic Leptospirosis, Icterus (Chronic Familial), Icterus Gravis Neonatorum, Icterus Intermittens Juvenalis, Idiopathic Alveolar Hypoventilation, Idiopathic Amyloidosis, Idiopathic Arteritis of Takayasu, Idiopathic Basal Ganglia Calcification (IBGC), Idiopathic Brachial Plexus Neuropathy, Idiopathic Cervical Dystonia, Idiopathic Dilatation of the Pulmonary Artery, Idiopathic Facial Palsy, Idiopathic Familial Hyperlipemia, Idiopathic Hypertrophic Subaortic Stenosis, Idiopathic Hypoproteinemia, Idiopathic Immunoglobulin Deficiency, Idiopathic Neonatal Hepatitis, Idiopathic Non-Specific Ulcerative Colitis, Idiopathic Peripheral Periphlebitis, Idiopathic Pulmonary Fibrosis, Idiopathic Refractory Sideroblastic Anemia, Idiopathic Renal Hematuria, Idiopathic Steatorrhea, Idiopathic Thrombocythemia, Idiopathic Thrombocytopenic Purpura, Idiopathic Thrombocytopenia Purpura (ITP), IDPA, IgA Nephropathy, IHSS, Ileitis, Ileocolitis, Illinois Type Amyloidosis, ILS, IM, IMD2, IMD5, Immune Defect due to Absence of Thymus, Immune Hemolytic Anemia Paroxysmal Cold, Immunodeficiency with Ataxia Telangiectasia, Immunodeficiency Cellular with Abnormal Immunoglobulin Synthesis, Immunodeficiency Common Variable Unclassifiable, Immunodeficiency with Hyper-IgM, Immunodeficiency with Leukopenia, Immunodeficiency-2, Immunodeficiency-5 (IMD5), Immunoglobulin Deficiency, Imperforate Anus, Imperforate Anus with Hand Foot and Ear Anomalies, Imperforate Nasolacrimal Duct and Premature Aging Syndrome, Impotent Neutrophil Syndrome, Inability To Open Mouth Completely And Short Finger-Flexor, INAD, Inborn Error of Urea Synthesis Arginase Type, Inborn Error of Urea Synthesis Arginino Succinic Type, Inborn Errors of Urea Synthesis Carbamyl Phosphate Type, Inborn Error of Urea Synthesis Citrullinemia Type, Inborn Errors of Urea Synthesis Glutamate Synthetase Type, INCL, Inclusion body myositis, Incomplete Atrioventricular Septal Defect, Incomplete Testicular Feminization, Incontinentia Pigmenti, Incontinenti Pigmenti Achromians, Index Finger Anomaly with Pierre Robin Syndrome, Indiana Type Amyloidosis (Type II), Indolent systemic mastocytosis, Infantile Acquired Aphasia, Infantile Autosomal Recessive Polycystic Kidney Disease, Infantile Beriberi, Infantile Cerebral Ganglioside, Infantile Cerebral Paralysis, Infantile Cystinosis, Infantile Epileptic, Infantile Fanconi Syndrome with Cystinosis, Infantile Finnish Type Neuronal Ceroid Lipofuscinosis, Infantile Gaucher Disease, Infantile Hypoglycemia, Infantile Hypophasphatasia, Infantile Lobar Emphysema, Infantile Myoclonic Encephalopathy, Infantile Myoclonic Encephalopathy and Polymyoclonia, Infantile Myofibromatosis, Infantile Necrotizing Encephalopathy, Infantile Neuronal Ceroid Lipofuscinosis, Infantile Neuroaxonal Dystrophy, Infantile Onset Schindler Disease, Infantile Phytanic Acid Storage Disease, Infantile Refsum Disease (IRD), Infantile Sipoidosis GM-2 Gangliosideosis (Type S), Infantile Sleep Apnea, Infantile Spasms, Infantile Spinal Muscular Atrophy (all types), Infantile Spinal Muscular Atrophy ALS, Infantile Spinal Muscular Atrophy Type I, Infantile Type Neuronal Ceroid Lipofuscinosis, Infectious Jaundice, Inflammatory Breast Cancer, Inflammatory Linear Nevus Sebaceous Syndrome, Iniencephaly, Insulin Resistant Acanthosis Nigricans, Insulin Lipodystrophy, Insulin dependent Diabetes, Intention Myoclonus, Intermediate Cystinosis, Intermediate Maple Syrup Urine Disease, Intermittent Ataxia with Pyruvate Dehydrogenase Deficiency, Intermittent Maple Syrup Urine Disease, Internal Hydrocephalus, Interstitial Cystitis, Interstitial Deletion of 4q Included, Intestinal Lipodystrophy, Intestinal Lipophagic Granulomatosis, Intestinal Lymphangiectasia, Intestinal Polyposis I, Intestinal Polyposis II, Intestinal Polyposis III, Intestinal Polyposis-Cutaneous Pigmentation Syndrome, Intestinal Pseudoobstruction with External Ophthalmoplegia, Intra-cranial Neoplasm, Intra-cranial Tumors, Intracranial Vascular Malformations, Intra-uterine Dwarfism, Intra-uterine Synechiae, Inverted Smile And Occult Neuropathic Bladder, Iowa Type Amyloidosis (Type IV), IP, IPA, Iridocorneal Endothelial Syndrome, Iridocorneal Endothelial (ICE) Syndrome Cogan-Resse Type, Iridogoniodysgenesis With Somatic Anomalies, Iris Atrophy with Corneal Edema and Glaucoma, Iris Nevus Syndrome, Iron Overload Anemia, Iron Overload Disease, Irritable Bowel Syndrome, Irritable Colon Syndrome, Isaacs Syndrome, Isaacs-Merten Syndrome, Ischemic Cardiomyopathy, Isolated Lissencephaly Sequence, Isoleucine 33 Amyloidosis, Isovaleric Acid CoA Dehydrogenase Deficiency, Isovaleric Acidaemia, Isovalericacidemia, Isovaleryl CoA Carboxylase Deficiency, ITO Hypomelanosis, ITO, ITP, IVA, Ivemark Syndrome, Iwanoff Cysts, Jackknife Convulsion, Jackson-Weiss Craniosynostosis, Jackson-Weiss Syndrome, Jacksonian Epilepsy, Jacobsen Syndrome, Jadassohn-Lewandowsky Syndrome, Jaffe-Lichenstein Disease, Jakob's Disease, Jakob-Creutzfeldt Disease, Janeway I, Janeway Dysgammaglobulinemia, Jansen Metaphyseal Dysostosis, Jansen Type Metaphyseal Chondrodysplasia, Jarcho-Levin Syndrome, Jaw-Winking, JBS, JDMS, Jegher's Syndrome, Jejunal Atresia, Jejunitis, Jejunoileitis, Jervell and Lange-Nielsen Syndrome, Jeune Syndrome, JMS, Job Syndrome, Job-Buckley Syndrome, Johanson-Blizzard Syndrome, John Dalton, Johnson-Stevens Disease, Jonston's Alopecia, Joseph's Disease, Joseph's Disease Type I, Joseph's Disease Type II, Joseph's Disease Type III, Joubert Syndrome, Joubert-Bolthauser Syndrome, JRA, Juberg Hayward Syndrome, Juberg-Marsidi Syndrome, Juberg-Marsidi Mental Retardation Syndrome, Jumping Frenchmen, Jumping Frenchmen of Maine, Juvenile Arthritis, Juvenile Autosomal Recessive Polycystic Kidney Disease, Juvenile Cystinosis, Juvenile (Childhood) Dermatomyositis (JDMS), Juvenile Diabetes, Juvenile Gaucher Disease, Juvenile Gout Choreoathetosis and Mental Retardation Syndrome, Juvenile Intestinal Malabsorption of Vit B12, Juvenile Intestinal Malabsorption of Vitamin B12, Juvenile Macular Degeneration, Juvenile Pernicious Anemia, Juvenile Retinoschisis, Juvenile Rheumatoid Arthritis, Juvenile Spinal Muscular Atrophy Included, Juvenile Spinal Muscular Atrophy ALS Included, Juvenile Spinal Muscular Atrophy Type III, Juxta-Articular Adiposis Dolorosa, Juxtaglomerular Hyperplasia, Kabuki Make-Up Syndrome, Kahler Disease, Kallmann Syndrome, Kanner Syndrome, Kanzaki Disease, Kaposi Disease, κ-Light Chain Deficiency, Karsch-Neugebauer Syndrome, Kartagener Syndrome-Chronic Sinobronchial Disease and Dextrocardia, Kartagener Triad, Kasabach-Merritt Syndrome, Kast Syndrome, Kawasaki Disease, Kawasaki Syndrome, KBG Syndrome, KD, Kearns-Sayre Disease, Kearns-Sayre Syndrome, Kennedy Disease, Kennedy Syndrome, Kennedy Type Spinal and Bulbar Muscular Atrophy, Kennedy-Stefanis Disease, Kenny Disease, Kenny Syndrome, Kenny Type Tubular Stenosis, Kenny-Caffe Syndrome, Keratitis Ichthyosis Deafness Syndrome, Keratoconus, Keratoconus Posticus Circumscriptus, Keratolysis, Keratolysis Exfoliativa Congenita, Keratolytic Winter Erythema, Keratomalacia, Keratosis Follicularis, Keratosis Follicularis Spinulosa Decalvans, Keratosis Follicularis Spinulosa Decalvans Ichthyosis, Keratosis Nigricans, Keratosis Palmoplantaris with Periodontopathia and Onychogryposis, Keratosis Palmoplantaris Congenital Pes Planus Onychogryposis Periodontosis Arachnodactyly, Keratosis Palmoplantaris Congenital, Pes Planus, Onychogryphosis, Periodontosis, Arachnodactyly, Acroosteolysis, Keratosis Rubra Figurata, Keratosis Seborrheica, Ketoacid Decarboxylase Deficiency, Ketoaciduria, Ketotic Glycinemia, KFS, KID Syndrome, Kidney Agenesis, Kidneys Cystic-Retinal Aplasia Joubert Syndrome, Killian Syndrome, Killian/Teschler-Nicola Syndrome, Kiloh-Nevin Syndrome III, Kinky Hair Disease, Kinsbourne Syndrome, Kleeblattschadel Deformity, Kleine-Levin Syndrome, Kleine-Levin Hibernation Syndrome, Klinefelter, Klippel-Feil Syndrome, Klippel-Feil Syndrome Type I, Klippel-Feil Syndrome Type II, Klippel-Feil Syndrome Type III, Klippel Trenaunay Syndrome, Klippel-Trenaunay-Weber Syndrome, Kluver-Bucy Syndrome, KMS, Kniest Dysplasia, Kniest Syndrome, Kobner's Disease, Koebberling-Dunnigan Syndrome, Kohlmeier-Degos Disease, Kok Disease, Korsakoff Psychosis, Korsakoff's Syndrome, Krabbe's Disease Included, Krabbe's Leukodystrophy, Kramer Syndrome, KSS, KTS, KTW Syndrome, Kufs Disease, Kugelberg-Welander Disease, Kugelberg-Welander Syndrome, Kussmaul-Landry Paralysis, KWS, L-3-Hydroxy-Acyl-CoA Dehydrogenase (LCHAD) Deficiency, Laband Syndrome, Labhart-Willi Syndrome, Labyrinthine Syndrome, Labyrinthine Hydrops, Lacrimo-Auriculo-Dento-Digital Syndrome, Lactase Isolated Intolerance, Lactase Deficiency, Lactation-Uterus Atrophy, Lactic Acidosis Leber Hereditary Optic Neuropathy, Lactic and Pyruvate Acidemia with Carbohydrate Sensitivity, Lactic and Pyruvate Acidemia with Episodic Ataxia and Weakness, Lactic and Pyruvate, Lactic Acidosis, Lactose Intolerance of Adulthood, Lactose Intolerance, Lactose Intolerance of Childhood, LADD Syndrome, LADD, Lafora Disease Included, Lafora Body Disease, Laki-Lorand Factor Deficiency, LAM, Lambert Type Ichthyosis, Lambert-Eaton Syndrome, Lambert-Eaton Myasthenic Syndrome, Lamellar Recessive Ichthyosis, Lamellar Ichthyosis, Lancereaux-Mathieu-Weil Spirochetosis, Landau-Kleffner Syndrome, Landouzy Dejerine Muscular Dystrophy, Landry Ascending Paralysis, Langer-Salidino Type Achondrogensis (Type II), Langer Giedion Syndrome, Langerhans-Cell Granulomatosis, Langerhans-Cell Histiocytosis (LCH), Large Atrial and Ventricular Defect, Laron Dwarfism, Laron Type Pituitary Dwarfism, Larsen Syndrome, Laryngeal Dystonia, Latah (Observed in Malaysia), Late Infantile Neuroaxonal Dystrophy, Late Infantile Neuroaxonal Dystrophy, Late Onset Cockayne Syndrome Type III (Type C), Late-Onset Dystonia, Late-Onset Immunoglobulin Deficiency, Late Onset Pelizaeus-Merzbacher Brain Sclerosis, Lattice Corneal Dystrophy, Lattice Dystrophy, Launois-Bensaude, Launois-Cleret Syndrome, Laurence Syndrome, Laurence-Moon Syndrome, Laurence-Moon/Bardet-Biedl, Lawrence-Seip Syndrome, LCA, LCAD Deficiency, LCAD, LCAD, LCADH Deficiency, LCH, LCHAD, LCPD, Le Jeune Syndrome, Leband Syndrome, Leber's Amaurosis, Leber's Congenital Amaurosis, Congenital Absence of the Rods and Cones, Leber's Congenital Tapetoretinal Degeneration, Leber's Congenital Tapetoretinal Dysplasia, Leber's Disease, Leber's Optic Atrophy, Leber's Optic Neuropathy, Left Ventricular Fibrosis, Leg Ulcer, Legg-Calve-Perthes Disease, Leigh's Disease, Leigh's Syndrome, Leigh's Syndrome (Subacute Necrotizing Encephalomyelopathy), Leigh Necrotizing Encephalopathy, Lennox-Gastaut Syndrome, Lentigio-Polypose-Digestive Syndrome, Lenz Dysmorphogenetic Syndrome, Lenz Dysplasia, Lenz Microphthalmia Syndrome, Lenz Syndrome, LEOPARD Syndrome, Leprechaunism, Leptomeningeal Angiomatosis, Leptospiral Jaundice, Leri-Weill Disease, Leri-Weil Dyschondrosteosis, Leri-Weil Syndrome, Lermoyez Syndrome, Leroy Disease, Lesch Nyhan Syndrome, Lethal Infantile Cardiomyopathy, Lethal Neonatal Dwarfism, Lethal Osteochondrodysplasia, Letterer-Siwe Disease, Leukocytic Anomaly Albinism, Leukocytic Inclusions with Platelet Abnormality, Leukodystrophy, Leukodystrophy with Rosenthal Fibers, Leukoencephalitis Periaxialis Concentric, Levine-Critchley Syndrome, Levulosuria, Levy-Hollister Syndrome, LGMD, LGS, LHON, LIC, Lichen Ruber Acuminatus, Lichen Acuminatus, Lichen Amyloidosis, Lichen Planus, Lichen Psoriasis, Lignac-Debre-Fanconi Syndrome, Lignac-Fanconi Syndrome, Ligneous Conjunctivitis, Limb-Girdle Muscular Dystrophy, Limb Malformations-Dento-Digital Syndrome, Limit Dextrinosis, Linear Nevoid Hypermelanosis, Linear Nevus Sebacous Syndrome, Linear Scleroderma, Linear Sebaceous Nevus Sequence, Linear Sebaceous Nevus Syndrome, Lingua Fissurata, Lingua Plicata, Lingua Scrotalis, Linguofacial Dyskinesia, Lip Pseudocleft-hemangiomatous Branchial Cyst Syndrome, Lipid Granulomatosis, Lipid Histiocytosis, Lipid Kerasin Type, Lipid Storage Disease, Lipid-Storage Myopathy Associated with SCAD Deficiency, Lipidosis Ganglioside Infantile, Lipoatrophic Diabetes Mellitus, Lipodystrophy, Lipoid Corneal Dystrophy, Lipoid Hyperplasia-Male Pseudohermaphroditism, Lipomatosis of Pancreas Congenital, Lipomucopolysaccharidosis Type I, Lipomyelomeningocele, Lipoprotein Lipase Deficiency Familial, LIS, LIS1, Lissencephaly 1, Lissencephaly Type T, Lissencephaly Variants With Agenesis of the Corpus Callosum Cerebellar Hypoplasia or Other Anomalies, Little Disease, Liver Phosphorylase Deficiency, LKS, LM Syndrome, Lobar Atrophy, Lobar Atrophy of the Brain, Lobar Holoprosencephaly, Lobar Tension Emphysema in Infancy, Lobstein Disease (Type I), Lobster Claw Deformity, Localized Epidermolysis Bullosa, Localized Lipodystrophy, Localized Neuritis of the Shoulder Girdle, Loeffler's Disease, Loeffler Endomyocardial Fibrosis with Eosinophilia, Loeffler Fibroplastic Parietal Endocarditis, Loken Syndrome, Loken-Senior Syndrome, Long-Chain 3-hydroxyacyl-CoA Dehydrogenase (LCHAD), Long Chain Acyl CoA Dehydrogenase Deficiency, Long-Chain Acyl-CoA Dehydrogenase (ACADL), Long-Chain Acyl-CoA Dehydrogenase Deficiency, Long QT Syndrome without Deafness, Lou Gehrig's Disease, Lou Gehrig's Disease Included, Louis-Bar Syndrome, Low Blood Sugar, Low-Density β Lipoprotein Deficiency, Low Imperforate Anus, Low Potassium Syndrome, Lowe syndrome, Lowe's Syndrome, Lowe-Bickel Syndrome, Lowe-Terry-MacLachlan Syndrome, LS, LTD, Lubs Syndrome, Luft Disease, Lumbar Canal Stenosis, Lumbar Spinal Stenosis, Lumbosacral Spinal Stenosis, Lundborg-Unverricht Disease, Lundborg-Unverricht Disease Included, Lupus, Lupus Erythematosus, Luschka-Magendie Foramina Atresia, Lyell Syndrome, Lyelles Syndrome, Lymphadenoid Goiter, Lymphangiectatic Protein-Losing Enteropathy, Lymphangioleiomatosis, Lymphangioleimyomatosis, Lymphangiomas, Lymphatic Malformations, Lynch Syndromes, Lynch Syndrome I, Lynch Syndrome II, Lysosomal α-N-Acetylgalactosaminidase Deficiency Schindler Type, Lysosomal Glycoaminoacid Storage Disease-Angiokeratoma Corporis Diffusum, Lysosomal Glucosidase Deficiency, MAA, Machado Disease, Machado-Joseph Disease, Macrencephaly, Macrocephaly, Macrocephaly Hemihypertrophy, Macrocephaly with Multiple Lipomas and Hemangiomata, Macrocephaly with Pseudo-papilledema and Multiple Hemangiomata, Macroglobulinemia, Macroglossia, Macroglossia-Omphalocele-Visceromegaly Syndrome, Macrostomia Ablepheron Syndrome, Macrothrombocytopenia Familial Bernard-Soulier Type, Macula Lutea Degeneration, Macular Amyloidosis, Macular Degeneration, Macular Degeneration Disciform, Macular Degeneration Senile, Macular Dystrophy, Macular Type Corneal Dystrophy, MAD, Madelung's Disease, Mafflucci Syndrome, Major Epilepsy, Malabsorption, Malabsorption-Ectodermal Dysplasia-Nasal Alar Hypoplasia, Maladie de Roger, Maladie de Tics, Male Malformation of Limbs and Kidneys, Male Turner Syndrome, Malignant Acanthosis, Malignant Acanthosis Nigricans, Malignant Astrocytoma, Malignant Atrophic Papulosis, Malignant Fever, Malignant Hyperphenylalaninemia, Malignant Hyperpyrexia, Malignant Hyperthermia, Malignant Melanoma, Malignant Tumors of the Central Nervous System, Mallory-Weiss Laceration, Mallory-Weiss Tear, Mallory-Weiss Syndrome, Mammary Paget's Disease, Mandibular Ameloblastoma, Mandibulofacial Dysostosis, Manic Depression Illness Disease, Mannosidosis, Map-Dot-Fingerprint Type Corneal Dystrophy, Maple Syrup Urine Disease, Marble Bones, Marchiafava-Micheli Syndrome, Marcus Gunn Jaw-Winking Syndrome, Marcus Gunn Phenomenon, Marcus Gunn Ptosis with Jaw-Winking, Marcus Gunn Syndrome, Marcus Gunn (Jaw-Winking) Syndrome, Marcus Gunn Ptosis (with Jaw-Winking), Marden-Walker Syndrome, Marden-Walker Type Connective Tissue Disorder, Marfan's Abiotrophy, Marfan-Achard Syndrome, Marfan Syndrome, Marfan's Syndrome I, Marfan's Variant, Marfanoid Hypermobility Syndrome, Marginal Corneal Dystrophy, Marie's Ataxia, Marie Disease, Marie-Sainton Disease, Marie Strumpell Disease, Marie-Strumpell Spondylitis, Marinesco-Sjogren Syndrome, Marinesco-Sjogren-Gorland Syndrome, Marker X Syndrome, Maroteaux Lamy Syndrome, Maroteaux Type Acromesomelic Dysplasia, Marshall's Ectodermal Dysplasias With Ocular and Hearing Defects, Marshall-Smith Syndrome, Marshall Syndrome, Marshall Type Deafness-Myopia-Cataract-Saddle Nose, Martin-Albright Syndrome, Martin-Bell Syndrome, Martorell Syndrome, MASA Syndrome, Massive Myoclonia, Mast Cell Leukemia, Mastocytosis, Mastocytosis With an Associated Hematologic Disorder, Maumenee Corneal Dystrophy, Maxillary Ameloblastoma, Maxillofacial Dysostosis, Maxillonasal Dysplasia, Maxillonasal Dysplasia Binder Type, Maxillopalpebral Synkinesis, May-Hegglin Anomaly, MCAD Deficiency, MCAD, McArdle Disease, McCune-Albright, MCD, McKusick Type Metaphyseal Chondrodysplasia, MCR, MCTD, Meckel Syndrome, Meckel-Gruber Syndrome, Median Cleft Face Syndrome, Mediterranean Anemia, Medium-Chain Acyl-CoA Dehydrogenase (ACADM), Medium Chain Acyl-CoA Dehydrogenase (MCAD) Deficiency, Medium-Chain Acyl-CoA Dehydrogenase Deficiency, Medullary Cystic Disease, Medullary Sponge Kidney, MEF, Megaesophagus, Megalencephaly, Megalencephaly with Hyaline Inclusion, Megalencephaly with Hyaline Panneuropathy, Megaloblastic Anemia, Megaloblastic Anemia of Pregnancy, Megalocornea-Mental Retardation Syndrome, Meier-Gorlin Syndrome, Meige's Lymphedema, Meige's Syndrome, Melanodermic Leukodystrophy, Melanoplakia-Intestinal Polyposis, Melanoplakia-Intestinal Polyposis, MELAS Syndrome, MELAS, Melkersson Syndrome, Melnick-Fraser Syndrome, Melnick-Needles Osteodysplasty, Melnick-Needles Syndrome, Membranous Lipodystrophy, Mendes Da Costa Syndrome, Meniere Disease, Ménière's Disease, Meningeal Capillary Angiomatosis, Menkes Disease, Menke's Syndrome I, Mental Retardation Aphasia Shuffling Gait Adducted Thumbs (MASA), Mental Retardation-Deafness-Skeletal Abnormalities-Coarse Face with Full Lips, Mental Retardation with Hypoplastic 5th Fingernails and Toenails, Mental Retardation with Osteocartilaginous Abnormalities, Mental Retardation-X-linked with Growth Delay-Deafness-Microgenitalism, Menzel Type OPCA, Mermaid Syndrome, MERRF, MERRF Syndrome, Merten-Singleton Syndrome, MES, Mesangial IGA Nephropathy, Mesenteric Lipodystrophy, Mesiodens-Cataract Syndrome, Mesodermal Dysmorphodystrophy, Mesomelic Dwarfism-Madelung Deformity, Metabolic Acidosis, Metachromatic Leukodystrophy, Metatarsus Varus, Metatropic Dwarfism. Syndrome, Metatropic Dysplasia, Metatropic Dysplasia I, Metatropic Dysplasia II, Methylmalonic Acidemia, Methylmalonic Aciduria, Meulengracht's Disease, MFD1, MG, MH, MHA, Micrencephaly, Microcephalic Primordial Dwarfism I, Microcephaly, Microcephaly-Hiatal Hernia-Nephrosis Galloway Type, Microcephaly-Hiatal Hernia-Nephrotic Syndrome, Microcystic Corneal Dystrophy, Microcythemia, Microlissencephaly, Microphthalmia, Microphthalmia or Anopthalmos with Associated Anomalies, Micropolygyria With Muscular Dystrophy, Microtia Absent, Patellae Micrognathia Syndrome, Microvillus Inclusion Disease, MID, Midsystolic-Click-Late Systolic Murmur Syndrome, Miescher's Type I Syndrome, Mikulicz Syndrome, Mikulicz-Radecki Syndrome, Mikulicz-Sjogren Syndrome, Mild Autosomal Recessive, Mild Intermediate Maple Syrup Urine Disease, Mild Maple Syrup Urine Disease, Miller Syndrome, Miller-Dieker Syndrome, Miller-Fisher Syndrome, Milroy Disease, Minkowski-Chauffard Syndrome, Minor Epilepsy, Minot-Von Willebrand Disease, Mirror-Image Dextrocardia, Mitochondrial β-Oxidation Disorders, Mitrochondrial and Cytosolic, Mitochondrial Cytopathy, Mitochondrial Cytopathy, Keam-Sayre Type, Mitochondrial Encephalopathy, Mitochondrial Encephalo Myopathy Lactic Acidosis and Strokelike Episodes, Mitochondrial Myopathy, Mitochondrial Myopathy Encephalopathy Lactic Acidosis Stroke-Like Episode, Mitochondrial PEPCK Deficiency, Mitral-valve prolapse, Mixed Apnea, Mixed Connective Tissue Disease, Mixed Hepatic Porphyria, Mixed Non-Fluent Aphasia, Mixed Sleep Apnea, Mixed Tonic and Clonic Torticollis, MJD, MKS, ML I, ML II, ML III, ML IV, ML Disorder Type I, ML Disorder Type II, ML Disorder Type III, ML Disorder Type IV, MLNS, M Syndrome, MND, MNGIE, MNS, Mobitz I, Mobitz II, Mobius Syndrome, Moebius Syndrome, Moersch-Woltmann Syndrome, Mohr Syndrome, Monilethrix, Monomodal Visual Amnesia, Mononeuritis Multiplex, Mononeuritis Peripheral, Mononeuropathy Peripheral, Monosomy 3p2, Monosomy 9p Partial, Monosomy 11q Partial, Monosomy 13q Partial, Monosomy 18q Syndrome, Monosomy X, Monostotic Fibrous Dysplasia, Morgagni-Turner-Albright Syndrome, Morphea, Morquio Disease, Morquio Syndrome, Morquio Syndrome A, Morquio Syndrome B, Morquio-Brailsford Syndrome, Morvan Disease, Mosaic Tetrasomy 9p, Motor Neuron Disease, Motor Neuron Syndrome, Motor Neurone Disease, Motoneuron Disease, Motoneurone Disease, Motor System Disease (Focal and Slow), Moya-Moya Disease, MPS, MPS I, MPS I H, NPS 1H/S Hurler/Scheie Syndrome, MPS I S Scheie Syndrome, MPS II, MPS IIA, MPS IIB, MPS II-AR Autosomal Recessive Hunter Syndrome, MPS II-XR, MPS II-XR Severe Autosomal Recessive, MPS III, MPS III A, B, C and D, Sanfiloppo A, MPS IV, MPS IV A and B Morquio A, MPS V, MPS VI Severe Intermediate Mild Maroteaux-Lamy, MPS VII Sly Syndrome, MPS VIII, MPS Disorder, MPS Disorder VI, MRS, MS, MSA, MSD, MSL, MSS, MSUD, MSUD, MSUD Type Ib, MSUD Type II, Mucocutaneous Lymph Node Syndrome, Mucolipidosis I, Mucolipidosis II, Mucolipidosis III, Mucolipidosis IV, Mucopolysaccharidosis, Mucopolysaccharidosis I-H, Mucopolysaccharidosis I-S, Mucopolysaccharidosis II, Mucopolysaccharidosis III, Mucopolysaccharidosis IV, Mucopolysaccharidosis VI, Mucopolysaccharidosis VII, Mucopolysaccharidosis Type I, Mucopolysaccharidosis Type II, Mucopolysaccharidosis Type III, Mucopolysaccharidosis Type VII, Mucosis, Mucosulfatidosis, Mucous Colitis, Mucoviscidosis, Mulibrey Dwarfism, Mulibrey Nanism Syndrome, Mullerian Duct Aplasia-Renal Aplasia-Cervicothoracic Somite Dysplasia, Mullerian Duct-Renal-Cervicothoracic-Upper Limb. Defects, Mullerian Duct and Renal Agenesis with Upper Limb and Rib Anomalies, Mullerian-Renal-Cervicothoracic Somite Abnormalities, Multi-Infarct Dementia Binswanger's Type, Multicentric Castleman's Disease, Multifocal Eosinophilic Granuloma, Multiple Acyl-CoA Dehydrogenase Deficiency, Multiple Acyl-CoA Dehydrogenase Deficiency/Glutaric Aciduria Type II, Multiple Angiomas and Endochondromas, Multiple Carboxylase Deficiency, Multiple Cartilaginous Enchondroses, Multiple Cartilaginous Exostoses, Multiple Enchondromatosis, Multiple Endocrine Deficiency Syndrome Type II, Multiple Epiphyseal Dysplasia, Multiple Exostoses, Multiple Exostoses Syndrome, Multiple Familial Polyposis, Multiple Lentigines Syndrome, Multiple Myeloma, Multiple Neuritis of the Shoulder Girdle, Multiple Osteochondromatosis, Multiple Peripheral Neuritis, Multiple Polyposis of the Colon, Multiple Pterygium Syndrome, Multiple Sclerosis, Multiple Sulfatase Deficiency, Multiple Symmetric Lipomatosis, Multiple System Atrophy, Multi-synostotic Osteodysgenesis, Multi-synostotic Osteodysgenesis with Long Bone Fractures, Mulvihill-Smith Syndrome, MURCS Association, Murk Jansen Type Metaphyseal Chondrodysplasia, Muscle Carnitine Deficiency, Muscle Core Disease, Muscle Phosphofructokinase Deficiency, Muscular Central Core Disease, Muscular Dystrophy, Muscular Dystrophy Classic X-linked Recessive, Muscular Dystrophy Congenital With Central Nervous System Involvement, Muscular Dystrophy Congenital Progressive with Mental Retardation, Muscular Dystrophy Facioscapulohumeral, Muscular Rheumatism, Muscular Rigidity-Progressive Spasm, Musculoskeletal Pain Syndrome, Mutilating Acropathy, Mutism, mvp, MVP, MWS, Myasthenia Gravis, Myasthenic Syndrome of Lambert-Eaton, Myelinoclastic Diffuse Sclerosis, Myelomatosis, Myhre Syndrome, Myoclonic Astatic Petit Mal Epilepsy, Myoclonic Dystonia, Myoclonic Encephalopathy of Infants, Myoclonic Epilepsy, Myoclonic Epilepsy Hartung Type, Myoclonus Epilepsy Associated with Ragged Red Fibers, Myoclonic Epilepsy and Ragged-Red Fiber Disease, Myoclonic Progressive Familial Epilepsy, Myoclonic Progressive Familial Epilepsy, Myoclonic Seizure, Myoclonus, Myoclonus Epilepsy, Myoencephalopathy Ragged-Red Fiber Disease, Myofibromatosis, Myofibromatosis Congenital, Myogenic Facio-Scapulo-Peroneal Syndrome, Myoneurogastointestinal Disorder and Encephalopathy, Myopathic Arthrogryposis Multiplex Congenita, Myopathic Carnitine Deficiency, Myopathy Central Fibrillar, Myopathy Congenital Nonprogressive, Myopathy Congenital Nonprogressive with Central Axis, myopathy with Deficiency of Carnitine Palmitoyltransferase, myopathy-Marinesco-Sjogren Syndrome, myopathy-Metabolic Carnitine Palmitoyltransderase Deficiency, Myopathy Mitochondrial-Encephalopathy-Lactic Acidosis-Stroke, Myopathy with Sarcoplasmic Bodies and Intermediate Filaments, Myophosphorylase Deficiency, Myositis Ossificans Progressive, Myotonia Atrophica, Myotonia Congenita, Myotonia Congenita Intermittens, Myotonic Dystrophy, Myotonic Myopathy Dwarfism Chondrodystrophy Ocular and Facial Anomalies, Myotubular Myopathy, Myotubular Myopathy X-linked, Myproic Acid, Myriachit (Observed in Siberia), Myxedema, N-Acetylglucosamine-1-Phosphotransferase Deficiency, N-Acetyl Glutamate Synthetase Deficiency, NADH-COQ Reductase Deficiency, Naegeli Ectodermal Dysplasias, Nager Syndrome, Nager Acrofacial Dysostosis Syndrome, Nager Syndrome, NAGS Deficiency, Nail Dystrophy-Deafness Syndrome, Nail Dysgenesis and Hypodontia, Nail-Patella Syndrome, Nance-Horan Syndrome, Nanocephalic Dwarfism, Nanocephaly, Nanophthalmia, Narcolepsy, Narcoleptic syndrome, NARP, Nasal-fronto-faciodysplasia, Nasal Alar Hypoplasia Hypothyroidism Pancreatic Achylia Congenital Deafness, Nasomaxillary Hypoplasia, Nasu Lipodystrophy, NBIA1, ND, NDI, NDP, Necrotizing Encephalomyelopathy of Leigh's, Necrotizing Respiratory Granulomatosis, Neill-Dingwall Syndrome, Nelson Syndrome, Nemaline myopathy, Neonatal Adrenoleukodystrophy, Neonatal Adrenoleukodystrophy (NALD), Neonatal Adrenoleukodystrophy (ALD), Neonatal Autosomal Recessive Polycystic Kidney Disease, Neonatal Dwarfism, Neonatal Hepatitis, Neonatal Hypoglycemia, Neonatal Lactose Intolerance, Neonatal Lymphedema due to Exudative Enteropathy, Neonatal Progeroid Syndrome, Neonatal Pseudo-Hydrocephalic Progeroid Syndrome of Wiedemann-Rautenstrauch, Neoplastic Arachnoiditis, Nephroblastom, Nephrogenic Diabetes Insipidus, Nephronophthesis Familial Juvenile, Nephropathic Cystinosis, Nephropathy-Pseudohermaphroditism-Wilms Tumor, Nephrosis-Microcephaly Syndrome, Nephrosis-Neuronal Dysmigration Syndrome, Nephrotic-Glycosuric-Dwarfism-Rickets-Hypophosphatemic Syndrome, Netherton Disease, Netherton Syndrome, Netherton Syndrome Ichthyosis, Nettleship Falls Syndrome (X-Linked), Neu-Laxova Syndrome, Neuhauser Syndrome, Neural-Tube Defects, Neuralgic Amyotrophy, Neuraminidase Deficiency, Neuraocutaneous Melanosis, Neurinoma of the Acoustic Nerve, Neurinoma, Neuroacanthocytosis, Neuroaxonral Dystrophy Schindler Type, Neurodegeneration with Brain Iron Accumulation Type 1 (NBIA1), Neurofibroma of the Acoustic Nerve, Neurogenic Arthrogryposis Multiplex Congenita, Neuromyelitis Optica, Neuromyotonia, Neuromyotonia, Focal, Neuromyotonia, Generalized, Familial, Neuromyotonia, Generalized, Sporadic, Neuronal Axonal Dystrophy Schindler Type, Neuronal Ceroid Lipofuscinosis Adult Type, Neuronal Ceroid Lipofuscinosis Juvenile Type, Neuronal Ceroid Lipofuscinosis Type 1, Neuronopathic Acute Gaucher Disease, Neuropathic Amyloidosis, Neuropathic Beriberi, Neuropathy Ataxia and Retinitis Pigmentosa, Neuropathy of Brachialpelxus Syndrome, Neuropathy Hereditary Sensory Type I, Neuropathy Hereditary Sensory Type II, Neutral Lipid Storage Disease, Nevii, Nevoid Basal Cell Carcinoma Syndrome, Nevus, Nevus Cavernosus, Nevus Comedonicus, Nevus Depigmentosus, Nevus Sebaceous of Jadassohn, Nezelof's Syndrome, Nezelof's Thymic Aplasia, Nezelof Type Severe Combined Immunodeficiency, NF, NF1, NF2, NF-1, NF-2, NHS, Niemann Pick Disease, Nieman Pick Disease Type A (acute neuronopathic form), Nieman Pick disease Type B, Nieman Pick Disease Type C (chronic neuronopathic form), Nieman Pick Disease Type D (Nova Scotia variant), Nieman Pick Disease Type E, Nieman Pick Disease Type F (sea-blue histiocyte disease), Night Blindness, Nigrospinodentatal Degeneration, Niikawakuroki Syndrome, NLS, NM, Noack Syndrome Type I, Nocturnal Myoclonus Hereditary Essential Myoclonus, Nodular Cornea Degeneration, Non-Bullous CIE, Non-Bullous Congenital Ichthyosiform Erythroderma, Non-Communicating Hydrocephalus, Non-Deletion Type α-Thalassemia/Mental Retardation syndrome, Non-Ketonic Hyperglycinemia Type I (NKHI), Non-Ketotic Hyperglycinemia, Non-Lipid Reticuloendotheliosis, Non-Neuronopathic Chronic Adult Gaucher Disease, Non-Scarring Epidermolysis Bullosa, Non-arteriosclerotic Cerebral Calcifications, Non-articular Rheumatism, Non-cerebral, Juvenile Gaucher Disease, Non-diabetic Glycosuria, Non-ischemic Cardiomyopathy, Non-ketotic Hypoglycemia and Carnitine Deficiency due to MCAD Deficiency, Non-ketotic Hypoglycemia Caused by Deficiency of Acyl-CoA Dehydrogenase, Non-ketotic Glycinemia, Nonne's Syndrome, Nonne-Milroy-Meige Syndrome, Nonopalescent Opalescent Dentine, Non-puerperal Galactorrhea-Amenorrhea, Non-secretory Myeloma, Non-spherocytic Hemolytic Anemia, Non-tropical Sprue, Noonan Syndrome, Norepinephrine, Normal Pressure Hydrocephalus, Norman-Roberts Syndrome, Norrbottnian Gaucher Disease, Norrie Disease, Norwegian Type Hereditary Cholestasis, NPD, NPS, NS, NSA, Nuchal Dystonia Dementia Syndrome, Nutritional Neuropathy, Nyhan Syndrome, OAV Spectrum, Obstructive Apnea, Obstructive Hydrocephalus, Obstructive Sleep Apnea, OCC Syndrome, Occlusive Thromboaortopathy, OCCS, Occult Intra-cranial Vascular Malformations, Occult Spinal Dysraphism Sequence, Ochoa Syndrome, Ochronosis, Ochronotic Arthritis, OCR, OCRL, Octocephaly, Ocular Albinism, Ocular Herpes, Ocular Myasthenia Gravis, Oculo-Auriculo-Vertebral Dysplasia, Oculo-Auriculo-Vertebral Spectrum, Oculo-Bucco-Genital Syndrome, Oculo-cerebral Syndrome with Hypopigmentation, Oculo-cerebrocutaneous Syndrome, Oculo-Cerebro-Renal, Oculo-cerebrorenal Dystrophy, Oculo-cerebrorenal Syndrome, Oculo-craniosomatic Syndrome (obsolete), Oculocutaneous Albinism, Oculocutaneous Albinism Chediak-Higashi Type, Oculo-Dento-Digital Dysplasia, Oculo-dentodigital Syndrome, Oculo-Dento-Osseous Dysplasia, Oculo Gastrointestinal Muscular Dystrophy, Oculo Gastrointestinal Muscular Dystrophy, Oculo-mandibulodyscephaly with Hypotrichosis, Oculo-mandibulo-facial Syndrome, Oculo-motor with Congenital Contractures and Muscle Atrophy, Oculosympathetic Palsy, ODD Syndrome, ODOD, Odontogenic Tumor, Odontotrichomelic Syndrome, OFD, OFD Syndrome, Ohio Type Amyloidosis (Type VII), OI, OI Congenita, OI Tarda, Oldfield Syndrome, Oligohydramnios Sequence, Oligophrenia Micropthalmos, Oligophrenic Polydystrophy, Olivopontocerebellar Atrophy, Olivopontocerebellar Atrophy with Dementia and Extrapyramidal Signs, Olivopontocerebellar Atrophy with Retinal Degeneration, Olivopontocerebellar Atrophy I, Olivopontocerebellar Atrophy II, Olivopontocerebellar Atrophy III, Olivopontocerebellar Atrophy IV, Olivopontocerebellar Atrophy V, Ollier Disease, Ollier Osteochondromatosis, Omphalocele-Visceromegaly-Macroglossia Syndrome, Ondine's Curse, Onion-Bulb Neuropathy, Onion Bulb Polyneuropathy, Onychoosteodysplasia, Onychotrichodysplasia with Neutropenia, OPCA, OPCA I, OPCA II, OPCA III, OPCA IV, OPCA V, OPD Syndrome, OPD Syndrome Type I, OPD Syndrome Type II, OPD I Syndrome, OPD II Syndrome, Ophthalmoarthropathy, Ophthalmoplegia-Intestinal Pseudo-obstruction, Ophthalmoplegia, Pigmentary Degeneration of the Retina and Cardiomyopathy, Ophthalmoplegia Plus Syndrome, Ophthalmoplegia Syndrome, Opitz BBB Syndrome, Opitz BBB/G Compound Syndrome, Opitz BBBG Syndrome, Opitz-Frias Syndrome, Opitz G Syndrome, Opitz G/BBB Syndrome, Opitz Hypertelorism-Hypospadias Syndrome, Opitz-Kaveggia Syndrome, Opitz Oculo-genito-laryngeal Syndrome, Opitz Trigonocephaly Syndrome, Opitz Syndrome, Opsoclonus, Opsoclonus-Myoclonus, Ophthalmoneuromyelitis, Optic Atrophy Polyneuropathy and Deafness, Optic Neuroencephalomyelopathy, Optic Neuromyelitis, Opticomyelitis, Optochiasmatic Arachnoiditis, Oral-Facial Clefts, Oral-Facial Dyskinesia, Oral Facial Dystonia, Oral-Facial-Digital Syndrome, Oral-Facial-Digital Syndrome Type I, Oral-Facial-Digital Syndrome I, Oral-Facial-Digital Syndrome II, Oral-Facial-Digital Syndrome III, Oral-Facial-Digital Syndrome IV, Orbital Cyst with Cerebral and Focal Dermal Malformations, Ornithine Carbamyl Transferase Deficiency, Ornithine Transcarbamylase Deficiency, Orocraniodigital Syndrome, Orofaciodigital Syndrome, Oromandibular Dystonia, Orthostatic Hypotension, Osler-Weber-Rendu Disease, Osseous-Oculo-Dento Dysplasia, Osseous-Oculo-Dento Dysplasia, Osteitis Deformans, Osteochondrodystrophy Deformans, Osteochondroplasia, Osteodysplasty of Melnick and Needles, Osteogenesis Imperfect, Osteogenesis Imperfecta, Osteogenesis Imperfecta Congenita, Osteogenesis Imperfecta Tarda, Osteohypertrophic Nevus Flammeus, Osteopathia Hyperostotica Scleroticans Multiplex Infantalis, Osteopathia Hyperostotica Scleroticans Multiplex Infantalis, Osteopathyrosis, Osteopetrosis, Qsteopetrosis Autosomal Dominant Adult Type, Osteopetrosis Autosomal Recessive Malignant Infantile Type, Osteopetrosis-Mild Autosomal Recessive Intermediate Type, Osteosclerosis Fragilis Generalisata, Osteosclerotic Myeloma, Ostium Primum Defect (endocardial cushion defects included), Ostium Secundum Defect, OTC Deficiency, Oto-Palato-Digital Syndrome, Oto-Palato-Digital Syndrome Type I, Oto-Palatal-Digital Syndrome Type II, Otodental Dysplasia, Otopalatodigital Syndrome, Otopalataldigital Syndrome Type II, Oudtshoorn Skin, Ovarian Dwarfism Turner Type, Ovary Aplasia Turner Type, OWR, Oxalosis, Oxidase Deficiency, Oxycephaly, Oxycephaly-Acrocephaly, P-V, PA, PAC, Pachyonychia Ichtyosiforme, Pachyonychia Congenita with Natal Teeth, Pachyonychia Congenita, Pachyonychia Congenita Keratosis Disseminata Circumscripta (follicularis), Pachyonychia Congenita Jadassohn-Lewandowsky Type, PAF with MSA, Paget's Disease, Paget's Disease of Bone, Paget's Disease of the Breast, Paget's Disease of the Nipple, Paget's Disease of the Nipple and Areola, Pagon Syndrome, Painful Ophthalmoplegia, PAIS, Palatal Myoclonus, Palato-Oto-Digital Syndrome, Palatal-Oto-Digital Syndrome Type I, Palatal-Oto-Digital Syndrome Type II, Pallister Syndrome, Pallister-Hall Syndrome, Pallister-Killian Mosaic Syndrome, Pallister Mosaic Aneuploidy, Pallister Mosaic Syndrome, Pallister Mosaic Syndrome Tetrasomy 12p, Pallister-W Syndrome, Palmoplantar Hyperkeratosis and Alopecia, Palsy, Pancreatic Fibrosis, Pancreatic Insufficiency and Bone Marrow Dysfunction, Pancreatic Ulcerogenic Tumor Syndrome, Panmyelophthisis, Panmyelopathy, Pantothenate Kinase Associated Neurodegeneration (PKAN), Papillon-Lefevre Syndrome, Papillotonic Psuedotabes, Paralysis Periodica Paramyotonica, Paralytic Beriberi, Paralytic Brachial Neuritis, Paramedian Lower Lip Pits-Popliteal Pyerygium Syndrome, Paramedian Diencephalic Syndrome, Paramyeloidosis, Paramyoclonus Multiple, Paramyotonia Congenita, Paramyotonia Congenita of Von Eulenburg, Parkinson's Disease, Paroxysmal Atrial Tachycardia, Paroxysmal Cold Hemoglobinuria, Paroxysmal Dystonia, Paroxysmal Dystonia Choreathetosis, Paroxysmal Kinesigenic Dystonia, Paroxysmal Nocturnal Hemoglobinuria, Paroxysmal Normal Hemoglobinuria, Paroxysmal Sleep, Parrot Syndrome, Parry Disease, Parry-Romberg Syndrome, Parsonage-Turner Syndrome, Partial Androgen Insensitivity Syndrome, Partial Deletion of the Short Arm of Chromosome 4, Partial Deletion of the Short Arm of Chromosome 5, Partial Deletion of Short Arm of Chromosome 9, Partial Duplication 3q Syndrome, Partial Duplication 15q Syndrome, Partial Facial Palsy With Urinary Abnormalities, Partial Gigantism of Hands and Feet Nevi-Hemihypertrophy-Macrocephaly, Partial Lipodystrophy, Partial Monosomy of Long Arm of Chromosome 11, Partial Monosomy of the Long Arm of Chromosome 13, Partial Spinal Sensory Syndrome, Partial Trisomy 11q, Partington Syndrome, PAT, Patent Ductus Arteriosus, Pathological Myoclonus, Pauciarticular-Onset Juvenile Arthritis, Paulitis, PBC, PBS, PC Deficiency, PC Deficiency Group A, PC Deficiency Group B, PC, Eulenburg Disease, PCC Deficiency, PCH, PCLD, PCT, PD, PDA, PDH Deficiency, Pearson Syndrome Pyruvate Carboxylase Deficiency, Pediatric Obstructive Sleep Apnea, Peeling Skin Syndrome, Pelizaeus-Merzbacher Disease, Pelizaeus-Merzbacher Brain Sclerosis, Pellagra-Cerebellar Ataxia-Renal Aminoaciduria Syndrome, Pelvic Pain Syndrome, Pemphigus Vulgaris, Pena Shokeir II Syndrome, Pena Shokeir Syndrome Type II, Penile Fibromatosis, Penile Fibrosis, Penile Induration, Penta X Syndrome, Pentalogy of Cantrell, Pentalogy Syndrome, Pentasomy X, PEPCK Deficiency, Pepper Syndrome, Perheentupa Syndrome, Periarticular Fibrositis, Pericardial Constriction with Growth Failure, Pericollagen Amyloidosis, Perinatal Polycystic Kidney Diseases, Perineal Anus, Periodic Amyloid Syndrome, Periodic Peritonitis Syndrome, Periodic Somnolence and Morbid Hunger, Periodic Syndrome, Peripheral Cystoid Degeneration of the Retina, Peripheral Dysostosis-Nasal Hypoplasia-Mental Retardation, Peripheral Neuritis, Peripheral Neuropathy, Peritoneopericardial Diaphragmatic Hernia, Pernicious Anemia, Peromelia with Micrognathia, Peroneal Muscular Atrophy, Peroneal Nerve Palsy, Peroutka Sneeze, Peroxisomal Acyl-CoA Oxidase, Peroxisomal β-Oxidation Disorders, Peroxisomal Bifunctional Enzyme, Peroxisomal Thiolase, Peroxisomal Thiolase Deficiency, Persistent Truncus Arteriosus, Perthes Disease, Petit Mal Epilepsy, Petit Mal Variant, Peutz-Jeghers Syndrome, Peutz-Touraine Syndrome, Peyronie Disease, Pfeiffer, Pfeiffer Syndrome Type I, PGA I, PGA II, PGA III, PGK, PH Type I, PH Type I, Pharyngeal Pouch Syndrome, PHD Short-Chain Acyl-CoA Dehydrogenase Deficiency, Phenylalanine Hydroxylase Deficiency, Phenylalaninemia, Phenylketonuria, Phenylpyruvic Oligophrenia, Phocomelia, Phocomelia Syndrome, Phosphoenolpyruvate Carboxykinase Deficiency, Phosphofructokinase Deficiency, Phosphoglycerate Kinase Deficiency, Phosphoglycerokinase, Phosphorylase 6 Kinase Deficiency, Phosphorylase Deficiency Glycogen Storage Disease, Phosphorylase Kinase Deficiency of Liver, Photic Sneeze Reflex, Photic Sneezing, Phototherapeutic Keratectomy, PHS, Physicist John Dalton, Phytanic Acid Storage Disease, Pi Phenotype ZZ, PI, Pick Disease of the Brain, Pick's Disease, Pickwickian Syndrome, Pierre Robin Anomalad, Pierre Robin Complex, Pierre Robin Sequence, Pierre Robin Syndrome, Pierre Robin Syndrome with Hyperphalangy and Clinodactyly, Pierre-Marie's Disease, Pigmentary Degeneration of Globus Pallidus Substantia Nigra Red Nucleus, Pili Torti and Nerve Deafness, Pili Torti-Sensorineural Hearing Loss, Pituitary Dwarfism II, Pituitary Tumor after Adrenalectomy, Pityriasis Pilaris, Pityriasis Rubra Pilaris, PJS, PKAN, PKD, PKD1, PKD2, PKD3, PKU, PKU1, Plagiocephaly, Plasma Cell Myeloma, Plasma Cell Leukemia, Plasma Thromboplastin Component Deficiency, Plasma Transglutaminase Deficiency, Plastic Induration Corpora Cavernosa, Plastic Induration of the Penis, PLD, Plicated Tongue, PLS, PMD, Pneumorenal Syndrome, PNH, PNM, PNP Deficiency, POD, POH, Poikiloderma Atrophicans and Cataract, Poikiloderma Congenital, Poland Anomaly, Poland Sequence, Poland Syndactyly, Poland Syndrome, Poliodystrophia Cerebri Progressiva, Polyarthritis Enterica, Polyarteritis Nodosa, Polyarticular-Onset Juvenile Arthritis Type I, Polyarticular-Onset Juvenile Arthritis Type II, Polychondritis, Polycystic Kidney Disease, Polycystic Kidney Disease Medullary Type, Polycystic Liver Disease, Polycystic Ovary Disease, Polycystic Renal Diseases, Polydactyly-Joubert Syndrome, Polydysplastic Epidermolysis Bullosa, Polydystrophia Oligophrenia, Polydystrophic Dwarfism, Polyglandular Autoimmune Syndrome Type III, Polyglandular Autoimmune Syndrome Type II, Polyglandular Autoimmune Syndrome Type I, Polyglandular Autoimmune Syndrome Type II, Polyglandular Syndromes, Polymorphic Macula Lutea Degeneration, Polymorphic Macular Degeneration, Polymorphism of Platelet Glycoprotien Ib, Polymorphous Corneal Dystrophy Hereditary, Polymyalgia Rheumatica, Polymyositis and Dermatomyositis, Primary A γ-globulinemia, Polyneuritis Peripheral, Polyneuropathy-Deafness-Optic Atrophy, Polyneuropathy Peripheral, Polyneuropathy and Polyradiculoneuropathy, Polyostotic Fibrous Dysplasia, Polyostotic Sclerosing Histiocytosis, Polyposis Familial, Polyposis Gardner Type, Polyposis Hamartomatous Intestinal, Polyposis-Osteomatosis-Epidermoid Cyst Syndrome, Polyposis Skin Pigmentation Alopecia and Fingernail Changes, Polyps and Spots Syndrome, Polyserositis Recurrent, Polysomy Y, Polysyndactyly with Peculiar Skull Shape, Polysyndactyly-Dysmorphic Craniofacies Greig Type, Pompe Disease, Pompe Disease, Popliteal Pterygium Syndrome, Porcupine Man, Porencephaly, Porencephaly, Porphobilinogen deaminase (PBG-D), Porphyria, Porphyria Acute Intermittent, Porphyria ALA-D, Porphyria Cutanea Tarda, Porphyria Cutanea Tarda Hereditaria, Porphyria Cutanea Tarda Symptomatica, Porphyria Hepatica Variegate, Porphyria Swedish Type, Porphyria Variegate, Porphyriam Acute Intermittent, Porphyrins, Porrigo Decalvans, Port Wine Stains, Portuguese Type Amyloidosis, Post-Infective Polyneuritis, Postanoxic Intention Myoclonus, Postaxial Acrofacial Dysostosis, Postaxial Polydactyly, Postencephalitic Intention Myoclonus, Posterior Corneal Dystrophy Hereditary, Posterior Thalamic Syndrome, Post-myelographic Arachnoiditis, Post-natal Cerebral Palsy, Post-operative Cholestasis, Postpartum Galactorrhea-Amenorrhea Syndrome, Postpartum Hypopituitarism, Postpartum Panhypopituitary Syndrome, Postpartum Panhypopituitarism, Postpartum Pituitary Necrosis, Postural Hypotension, Potassium-Losing Nephritis, Potassium Loss Syndrome, Potter Type I-Infantile Polycystic Kidney Diseases, Potter Type III Polycystic Kidney Disease, PPH, PPS, Prader-Willi Syndrome, Prader-Labhart-Willi Fancone Syndrome, Prealbumin Tyr-77 Amyloidosis, Pre-excitation Syndrome, Pregnenolone Deficiency, Premature Atrial Contractions, Premature Senility Syndrome, Premature Supraventricular Contractions, Premature Ventricular Complexes, Pre-natal or Con-natal Neuroaxonal Dystrophy, Pre-senile Dementia, Pre-senile Macula Lutea Retinae Degeneration, Primary Adrenal Insufficiency, Primary A γ-globulinemias, Primary Aldosteronism, Primary Alveolar Hypoventilation, Primary Amyloidosis, Primary Anemia, Primary Beriberi, Primary Biliary, Primary Biliary Cirrhosis, Primary Brown Syndrome, Primary Carnitine Deficiency, Primary Central Hypoventilation Syndrome, Primary Ciliary Dyskinesia Kartagener Type, Primary Cutaneous Amyloidosis, Primary Dystonia, Primary Failure Adrenocortical Insufficiency, Primary Familial Hypoplasia of the Maxilla, Primary Hemochromatosis, Primary Hyperhidrosis, Primary Hyperoxaluria [Type I], Primary Hyperoxaluria Type I (PHI), Primary Hyperoxaluria Type I, Primary Hyperoxaluria Type II, Primary Hyperoxaluria Type III, Primary Hypogonadism, Primary Intestinal Lymphangiectasia, Primary Lateral Sclerosis, Primary Non-hereditary Amyloidosis, Primary Obliterative Pulmonary Vascular Disease, Primary Progressive Multiple Sclerosis, Primary Pulmonary Hypertension, Primary Reading Disability, Primary Renal Glycosuria, Primary Sclerosing Cholangitis, Primary Thrombocythemia, Primary Tumors of Central Nervous System, Primary Visual Agnosia, Proctocolitis Idiopathic, Proctocolitis Idiopathic, Progeria of Adulthood, Progeria of Childhood, Progeroid Nanism, Progeriod Short Stature with Pigmented Nevi, Progeroid Syndrome of De Barsy, Progressive Autonomic Failure with Multiple System Atrophy, Progressive Bulbar Palsy, Progressive Bulbar Palsy Included, Progressive Cardiomyopathic Lentiginosis, Progressive Cerebellar Ataxia Familial, Progressive Cerebral Poliodystrophy, Progressive Choroidal Atrophy, Progressive Diaphyseal Dysplasia, Progressive Facial Hemiatrophy, Progressive Familial Myoclonic Epilepsy, Progressive Hemifacial Atrophy, Progressive Hypoerythemia, Progressive Infantile Poliodystrophy, Progressive Lenticular Degeneration, Progressive Lipodystrophy, Progressive Muscular Dystrophy of Childhood, Progressive Myoclonic Epilepsy, Progressive Osseous Heteroplasia, Progressive Pallid Degeneration Syndrome, Progressive Spinobulbar Muscular Atrophy, Progressive Supranuclear Palsy, Progressive Systemic Sclerosis, Progressive Tapetochoroidal Dystrophy, Proline Oxidase Deficiency, Propionic Acidemia, Propionic Acidemia Type I (PCCA Deficiency), Propionic Acidemia Type II (PCCB Deficiency), Propionyl CoA Carboxylase Deficiency, Protanomaly, Protanopia, Protein-Losing Enteropathy Secondary to Congestive Heart Failure, Proteus Syndrome, Proximal Deletion of 4q Included, PRP, PRS, Prune Belly Syndrome, PS, Pseudo-Hurler Polydystrophy, Pseudo-Polydystrophy, Pseudoacanthosis Nigricans, Pseudoachondroplasia, Pseudocholinesterase Deficiency, Pseudogout Familial, Pseudohemophilia, Pseudohermaphroditism, Pseudohermaphroditism-Nephron Disorder-Wilm's Tumor, Pseudohypertrophic Muscular Dystrophy, Pseudohypoparathyroidism, Pseudohypophosphatasia, Pseudopolydystrophy, Pseudothalidomide Syndrome, Pseudoxanthoma Elasticum, Psoriasis, Psorospermosis Follicularis, PSP, PSS, Psychomotor Convulsion, Psychomotor Epilepsy, Psychomotor Equivalent Epilepsy, PTC Deficiency, Pterygium, Pterygium Colli Syndrome, Pterygium Universale, Pterygolymphangiectasia, Pulmonary Atresia, Pulmonary Lymphangiomyomatosis, Pulmonary Stenosis, Pulmonic Stenosis-Ventricular Septal Defect, Pulp Stones, Pulpal Dysplasia, Pulseless Disease, Pure Alymphocytosis, Pure Cutaneous Histiocytosis, Purine Nucleoside Phosphorylase Deficiency, Purpura Hemorrhagica, Purtilo Syndrome, PXE, PXE Dominant Type, PXE Recessive Type, Pycnodysostosis, Pyknodysostosis, Pyknoepilepsy, Pyroglutamic Aciduria, Pyroglutamicaciduria, Pyrroline. Carboxylate Dehydrogenase Deficiency, Pyruvate Carboxylase Deficiency, Pyruvate Carboxylase Deficiency Group A, Pyruvate Carboxylase Deficiency Group B, Pyruvate Dehydrogenase Deficiency, Pyruvate Kinase Deficiency, q25-qter, q26 or q27-qter, q31 or 32-qter, QT Prolongation with Extracellular Hypohypocalcinemia, QT Prolongation without Congenital Deafness, QT Prolonged with Congenital Deafness, Quadriparesis of Cerebral Palsy, Quadriplegia of Cerebral Palsy, Quantal Squander, Quantal Squander, r4, r6, r14, r 18, r21, r22, Rachischisis Posterior, Radial Aplasia-Amegakaryocytic Thrombocytopenia, Radial Aplasia-Thrombocytopenia Syndrome, Radial Nerve Palsy, Radicular Neuropathy Sensory, Radicular Neuropathy Sensory Recessive, Radicular Dentin Dysplasia, Rapid-Onset Dystonia-Parkinsonism, Rapp-Hodgkin Syndrome, Rapp-Hodgkin (hypohidrotic) Ectodermal Dysplasia syndrome, Rapp-Hodgkin Hypohidrotic Ectodermal Dysplasias, Rare Hereditary Ataxia With Polyneuritic Changes and Deafness Caused by a Defect in the Enzyme Phytanic Acid Hydroxylase, Rautenstrauch-Wiedemann Syndrome, Rautenstrauch-Wiedemann Type Neonatal Progeria, Raynaud's Phenomenon, RDP, Reactive Functional Hypoglycemia, Reactive Hypoglycemia Secondary to Mild Diabetes, Recessive Type Kenny-Caffe Syndrome, Recklin Recessive Type Myotonia Congenita, Recklinghausen Disease, Rectoperineal Fistula, Recurrent Vomiting, Reflex Neurovascular Dystrophy, Reflex Sympathetic Dystrophy Syndrome, Refractive Errors, Refractory Anemia, Refrigeration Palsy, Refsum Disease, Refsum's Disease, Regional Enteritis, Reid-Barlow's syndrome, Reifenstein Syndrome, Reiger Anomaly-Growth Retardation, Reiger Syndrome, Reimann Periodic Disease, Reimann's Syndrome, Reis-Bucklers Corneal Dystrophy, Reiter's Syndrome, Relapsing Guillain-Barre Syndrome, Relapsing-Remitting Multiple Sclerosis, Renal Agenesis, Renal Dysplasia-Blindness Hereditary, Renal Dysplasia-Retinal Aplasia Loken-Senior Type, Renal Glycosuria, Renal Glycosuria Type A, Renal Glycosuria Type B, Renal Glycosuria Type O, Renal-Oculocerebrodystrophy, Renal-Retinal Dysplasia with Medullary Cystic Disease, Renal-Retinal Dystrophy Familial, Renal-Retinal Syndrome, Rendu-Osler-Weber Syndrome, Respiratory Acidosis, Respiratory Chain Disorders, Respiratory Myoclonus, Restless Legs Syndrome, Restrictive Cardiomyopathy, Retention Hyperlipemia, Rethore Syndrome (obsolete), Reticular Dysgenesis, Retinal Aplastic-Cystic Kidneys-Joubert Syndrome, Retinal Cone Degeneration, Retinal Cone Dystrophy, Retinal Cone-Rod Dystrophy, Retinitis Pigmentosa, Retinitis Pigmentosa and Congenital Deafness, Retinoblastoma, Retinol Deficiency, Retinoschisis, Retinoschisis Juvenile, Retraction Syndrome, Retrobulbar Neuropathy, Retrolenticular Syndrome, Rett Syndrome, Reverse Coarction, Reye's Syndrome, RGS, Rh Blood Factors, Rh Disease, Rh Factor Incompatibility, Rh Incompatibility, Rhesus Incompatibility, Rheumatic Fever, Rheumatoid Arthritis, Rheumatoid Myositis, Rhinosinusogenic Cerebral Arachnoiditis, Rhizomelic Chondrodysplasia Punctata (RCDP), Acatalasemia, Classical Refsum Disease, RHS, Rhythmical Myoclonus, Rib Gap Defects with Micrognathia, Ribbing Disease (obsolete), Ribbing Disease, Richner-Hanhart Syndrome, Rieger Syndrome, Rieter's Syndrome, Right Ventricular Fibrosis, Riley-Day Syndrome, Riley-Smith Syndrome, Ring Chromosome 14, Ring Chromosome 18, Ring 4, Ring 4 Chromosome, Ring 6, Ring 6 Chromosome, Ring 9, Ring 9 Chromosome R₉, Ring 14, Ring 15, Ring 15 Chromosome (mosaic pattern), Ring 18, Ring Chromosome 18, Ring 21, Ring 21 Chromosome, Ring 22, Ring 22 Chromosome, Ritter Disease, Ritter-Lyell Syndrome, RLS, RMSS, Roberts SC-Phocomelia Syndrome, Roberts Syndrome, Roberts Tetraphocomelia Syndrome, Robertson's Ectodermial Dysplasias, Robin Anomalad, Robin Sequence, Robin Syndrome, Robinow Dwarfism, Robinow Syndrome, Robinow Syndrome Dominant Form, Robinow Syndrome Recessive Form, Rod Myopathy, Roger Disease, Rokitansky's Disease, Romano-Ward Syndrome, Romberg Syndrome, Rootless Teeth, Rosenberg-Chutorian Syndrome, Rosewater Syndrome, Rosselli-Gulienatti Syndrome, Rothmund-Thonison Syndrome, Roussy-Levy Syndrome, RP, RS X-Linked, RS, RSDS, RSH Syndrome, RSS, RSTS, RTS, Rubella Congenital, Rubinstein Syndrome, Rubinstein-Taybi Syndrome, Rubinstein Taybi Broad Thumb-Hallux Syndrome, Rufous Albinism, Ruhr's Syndrome, Russell's Diencephalic Cachexia, Russell's Syndrome, Russell-Silver Dwarfism, Russell-Silver Syndrome, Russell-Silver Syndrome X-linked, Ruvalcaba-Myhre-Smith syndrome (RMSS), Ruvalcaba Syndrome, Ruvalcaba Type Osseous Dysplasia with Mental Retardation, Sacral Regression, Sacral Agenesis Congenital, SAE, Saethre-Chotzen Syndrome, Sakati, Sakati Syndrome, Sakati-Nyhan Syndrome, Salaam Spasms, Salivosudoriparous Syndrome, Salzman Nodular Corneal Dystrophy, Sandhoff Disease, Sanfilippo Syndrome, Sanfilippo Type A, Sanfilippo Type B, Santavuori Disease, Santavuori-Haltia Disease, Sarcoid of Boeck, Sarcoidosis, Sathre-chotzen, Saturday Night Palsy, SBMA, SC Phocomelia Syndrome, SC Syndrome, SCA 3, SCAD Deficiency, SCAD Deficiency Adult-Onset Localized, SCAD Deficiency Congenital Generalized, SCAD, SCADH Deficiency, Scalded Skin Syndrome, Scalp Defect Congenital, Scaphocephaly, Scapula Elevata, Scapuloperoneal Myopathy, Scapuloperoneal Muscular Dystrophy, Scapuloperoneal Syndrome Myopathic Type, Scarring Bullosa, SCHAD, Schaumann's Disease, Scheie Syndrome, Schereshevkii-Turner Syndrome, Schilder Disease, Schilder Encephalitis, Schilder's Disease, Schindler Disease Type I (Infantile Onset), Schindler Disease Infantile Onset, Schindler Disease, Schindler Disease Type II (Adult Onset), Schinzel Syndrome, Schinzel-Giedion Syndrome, Schinzel Acrocallosal Syndrome, Schinzel-Giedion Midface-Retraction Syndrome, Schizencephaly, Schmid Type Metaphyseal Chondrodysplasia, Schmid Metaphyseal Dysostosis, Schmid-Fraccaro Syndrome, Schmidt Syndrome, Schopf-Schultz-Passarge Syndrome, Schueller-Christian Disease, Schut-Haymaker Type, Schwartz-Jampel-Aberfeld Syndrome, Schwartz-Jampel Syndrome Types 1A and 1B, Schwartz-Jampel Syndrome Type 2, SCID, Scleroderma, Sclerosis Familial Progressive Systemic, Sclerosis Diffuse Familial Brain, Scott Cranio-digital Syndrome With Mental Retardation, Scrotal Tongue, SCS, SD, SDS, SDYS, Seasonal Conjunctivitis, Sebaceous Nevus Syndrome, Sebaceous nevus, Seborrheic Keratosis, Seborrheic Warts, Seckel Syndrome, Seckel Type Dwarfism, Second Degree Congenital Heart Block, Secondary Amyloidosis, Secondary Blepharospasm, Secondary Non-tropical Sprue, Secondary Brown Syndrome, Secondary Beriberi, Secondary Generalized Amyloidosis, Secondary Dystonia, Secretory Component Deficiency, Secretory IgA Deficiency, SED Tarda, SED Congenital, SEDC, Segmental Linear Achromic Nevus, Segmental Dystonia, Segmental Myoclonus, Seip Syndrome, Seitelberger Disease, Seizures, Selective Deficiency of IgG Sub-classes, Selective Mutism, Selective Deficiency of IgG Sub-class, Selective IgM Deficiency, Selective Mutism, Selective IgA Deficiency, Self-Healing Histiocytosis, Semi-lobar Holoprosencephaly, Seminiferous Tubule Dysgenesis, Senile Retinoschisis, Senile Warts, Senior-Loken Syndrome, Sensory Neuropathy Hereditary Type I, Sensory Neuropathy Hereditary Type II, Sensory Radicular Neuropathy, Sensory Radicular Neuropathy Recessive Sepsis, Septic Progressive Granulomatosis, Septo-Optic Dysplasia, Serous Circumscribed Meningitis, Serum Protease Inhibitor Deficiency, Serum Carnosinase Deficiency, Setleis Syndrome, Severe Combined Immunodeficiency, Severe Combined Immunodeficiency with Adenosine Deaminase Deficiency, Severe Combined Immunodeficiency (SCID), Sex Reversal, Sexual Infantilism, SGB Syndrome, Sheehan Syndrome, Shields Type Dentinogenesis Imperfecta, Shingles, Varicella-Zoster Virus, Ship Beriberi, SHORT Syndrome, Short Arm 18 Deletion Syndrome, Short Chain Acyl CoA Dehydrogenase Deficiency, Short Chain Acyl-CoA Dehydrogenase (SCAD) Deficiency, Short Stature and Facial Telangiectasis, Short Stature Facial/Skeletal Anomalies-Retardation-Macrodontia, Short Stature-Hyperextensibility-Rieger Anomaly-Teething Delay, Short Stature-Onychodysplasia, Short Stature Telangiectatic Erythema of the Face, SHORT Syndrome, Shoshin Beriberi, Shoulder Girdle Syndrome, Shprintzen-Goldberg Syndrome, Shulman Syndrome, Shwachman-Bodian Syndrome, Shwachman-Diamond Syndrome, Shwachman Syndrome, Shwachman-Diamond-Oski Syndrome, Shy Drager Syndrome, Shy-Magee Syndrome, SI Deficiency, Sialidase Deficiency, Sialidosis Type I Juvenile, Sialidosis Type II Infantile, Sialidosis, Sialolipidosis, Sick Sinus Syndrome, Sickle Cell Anemia, Sickle Cell Disease, Sickle Cell-Hemoglobin C Disease, Sickle Cell-Hemoglobin D Disease, Sickle Cell-Thalassemia Disease, Sickle Cell Trait, Sideroblastic Anemias, Sideroblastic Anemia, Sideroblastosis, SIDS, Siegel-Cattan-Mamou Syndrome, Siemens-Bloch Type Pigmented Dermatosis, Siemens Syndrome, Siewerling-Creutzfeldt Disease, Siewert Syndrome, Silver Syndrome, Silver-Russell Dwarfism, Silver-Russell Syndrome, Simmond's Disease, Simons Syndrome, Simplex Epidermolysis Bullosa, Simpson Dysmorphia Syndrome, Simpson-Golabi-Behmel Syndrome, Sinding-Larsen-Johansson Disease, Singleton-Merten Syndrome, Sinus Arrhythmia, Sinus Venosus, Sinus Tachycardia, Sirenomelia Sequence, Sirenomelus, Situs Inversus Bronchiectasis and Sinusitis, SJA Syndrome, Sjogren Larsson Syndrome Ichthyosis, Sjogren Syndrome, Sjögren's Syndrome, SJS, Skeletal Dysplasia, Skeletal Dysplasia Weismann Netter Stuhl Type, Skin Peeling Syndrome, Skin Neoplasms, Skull Asymmetry and Mild Retardation, Skull Asymmetry and Mild Syndactyly, SLE, Sleep Epilepsy, Sleep Apnea, SLO, Sly Syndrome, SMA, SMA Infantile Acute Form, SMA I, SMA III, SMA Type I, SMA Type II, SMA Type III, SMA3, SMAX1; SMCR, Smith Lemli Opitz Syndrome, Smith Magenis Syndrome, Smith-Magenis Chromosome Region, Smith-McCort Dwarfism, Smith-Opitz-Inborn Syndrome, Smith Disease, Smoldering Myeloma, SMS, SNE, Sneezing From Light Exposure, Sodium Valproate, Solitary Plasmacytoma of Bone, Sorsby Disease, Sotos Syndrome, Souques-Charcot Syndrome, South African Genetic Porphyria, Spasmodic Dysphonia, Spasmodic Torticollis, Spasmodic Wryneck, Spastic Cerebral Palsy, Spastic Colon, Spastic Dysphonia, Spastic Paraplegia, SPD Calcinosis, Specific Antibody Deficiency with Normal Immunoglobulins, Specific Reading Disability, SPH2, Spherocytic Anemia, Spherocytosis, Spherophakia-Brachymorphia Syndrome, Sphingomyelin Lipidosis, Sphingomyelinase Deficiency, Spider Fingers, Spielmeyer-Vogt Disease, Spielmeyer-Vogt-Batten Syndrome, Spina Bifida, Spina Bifida Aperta, Spinal Arachnoiditis, Spinal Arteriovenous Malformation, Spinal Ataxia Hereditofanilial, Spinal and Bulbar Muscular Atrophy, Spinal Diffuse Idiopathic Skeletal Hyperostosis, Spinal DISH, Spinal Muscular Atrophy, Spinal Muscular Atrophy All Types, Spinal Muscular Atrophy Type ALS, Spinal Muscular Atrophy-Hypertrophy of the Calves, Spinal Muscular Atrophy Type I, Spinal Muscular Atrophy Type III, Spinal Muscular Atrophy-Hypertrophy of the Calves, Spinal Ossifying Arachnoiditis, Spinal Stenosis, Spinocerebellar Ataxia, Spinocerebellar Atrophy Type I, Spiuocerebellar Ataxia Type I (SCA1), Spinocerebellar Ataxia Type II (SCAII), Spinocerebellar Ataxia Type III (SCAIII), Spinocerebellar Ataxia Type IV (SCAIV), Spinocerebellar Ataxia Type V (SCAV), Spinocerebellar Ataxia Type VI (SCAVI), Spinocerebellar Ataxia Type VII (SCAVII), Spirochetal Jaundice, Splenic Agenesis Syndrome, Splenic Ptosis, Splenoptosis, Split Hand Deformity-Mandibulofacial Dysostosis, Split Hand Deformity, Spondyloarthritis, Spondylocostal Dysplasia-Type I, Spondyloepiphyseal Dysplasia Tarda, Spondylothoracic Dysplasia, Spondylotic Caudal Radiculopathy, Sponge Kidney, Spongioblastoma Multiforme, Spontaneous Hypoglycemia, Sprengel Deformity, Spring Ophthalmia, SRS, ST, Stale Fish Syndrome, Staphyloccal Scalded Skin Syndrome, Stargardt's Disease, Startle Disease, Status Epilepticus, Steele-Richardson-Olszewski Syndrome, Steely Hair Disease, Stein-Leventhal Syndrome, Steinert Disease, Stengel's Syndrome, Stengel-Batten-Mayou-Spielmeyer-Vogt-Stock Disease, Stenosing Cholangitis, Stenosis of the Lumbar Vertebral Canal, Stenosis, Steroid Sulfatase Deficiency, Stevanovic's Ectodermal Dysplasias, Stevens Johnson Syndrome, STGD, Stickler Syndrome, Stiff-Man Syndrome, Stiff Person Syndrome, Still's Disease, Stilling-Turk-Duane Syndrome, Stillis Disease, Stimulus-Sensitive Myoclonus, Stone Man Syndrome, Stone Man, Streeter Anomaly, Striatonigral Degeneration Autosomal Dominant Type, Striopallidodentate Calcinosis, Stroma, Descemet's Membrane, Stromal Corneal Dystrophy, Struma Lymphomatosa, Sturge-Kalischer-Weber Syndrome, Sturge Weber Syndrome, Sturge-Weber Phakomatosis, Sub-acute Necrotizing Encephalomyelopathy, Sub-acute Spongiform Encephalopathy, Sub-acute Necrotizing Encephalopathy, Sub-acute Sarcoidosis, Sub-acute Neuronopathic, Sub-aortic Stenosis, Subcortical Arteriosclerotic Encephalopathy, Subendocardial Sclerosis, Succinylcholine Sensitivity, Sucrase-Isomaltase Deficiency Congenital, Sucrose-Isomaltose Malabsorption Congenital, Sucrose Intolerance Congenital, Sudanophilic Leukodystrophy ADL, Sudanophilic Leukodystrophy Pelizaeus-Merzbacher Type, Sudanophilic Leukodystrophy Included, Sudden Infant Death Syndrome, Sudeck's Atrophy, Sugio-Kajii Syndrome, Summerskill Syndrome, Summit Acrocephalosyndactyly, Summitt's Acrocephalosyndactyly, Summitt Syndrome, Superior Oblique Tendon Sheath Syndrome, Suprarenal Glands, Supravalvular Aortic Stenosis, Supraventricular Tachycardia, Surdicardiac Syndrome, Surdocardiac Syndrome, SVT, Sweat Gland Abscess, Sweating Gustatory Syndrome, Sweet Syndrome, Swiss Cheese Cartilage Syndrome, Syndactylic Oxycephaly, Syndactyly Type I with Microcephaly and Mental Retardation, Syndromatic Hepatic Ductular Hypoplasia, Syringomyelia, Systemic Aleukemic Reticuloendotheliosis, Systemic Amyloidosis, Systemic Carnitine Deficiency, Systemic Elastorrhexis, Systemic Lupus Erythematosus, Systemic Mast Cell Disease, Systemic Mastocytosis, Systemic-Onset Juvenile Arthritis, Systemic Sclerosis, Systopic Spleen, T-Lymphocyte Deficiency, Tachyalimentation Hypoglycemia, Tachycardia, Takahara Syndrome, Takayasu Disease, Takayasu Arteritis, Talipes Calcaneus, Talipes Equinovarus, Talipes Equinus, Talipes Varus, Talipes Valgus, Tandem Spinal Stenosis, Tangier Disease, Tapetoretinal Degeneration, TAR Syndrome, Tardive Dystonia, Tardive Muscular Dystrophy, Tardive Dyskinesia, Tardive Oral Dyskinesia, Tardive Dystonia, Tardy Ulnar Palsy, Target Cell Anemia, Tarsomegaly, Tarui Disease, TAS Midline Defects Included, TAS Midline Defect, Tay Sachs Sphingolipidosis, Tay Sachs Disease, Tay Syndrome Ichthyosis, Tay Sachs Sphingolipidosis, Tay Syndrome Ichthyosis, Taybi Syndrome Type I, Taybi Syndrome, TCD, TCOF1, TCS, TD, TDO Syndrome, TDO-I, TDO-II, TDO-III, Telangiectasis, Telecanthus with Associated Abnormalities, Telecanthus-Hypospadias Syndrome, Temporal Lobe Epilepsy, Temporal Arteritis/Giant Cell Arteritis, Temporal Arteritis, TEN, Tendon Sheath Adherence Superior Obliqu, Tension Myalgia, Terminal Deletion of 4q Included, Terrian Corneal Dystrophy, Teschler-Nicola/Killian Syndrome, Tethered Spinal Cord Syndrome, Tethered Cord Malformation Sequence, Tethered Cord Syndrome, Tethered Cervical. Spinal Cord Syndrome, Tetrahydrobiopterin Deficiencies, Tetrahydrobiopterin Deficiencies, Tetralogy of Fallot, Tetraphocomelia-Thrombocytopenia Syndrome, Tetrasomy Short Arm of Chromosome 9, Tetrasomy 9p, Tetrasomy Short Arm of Chromosome 18, Thalamic Syndrome, Thalamic Pain Syndrome, Thalamic Hyperesthetic Anesthesia, Thalassemia Intermedia, Thalassemia Minor, Thalassemia Major, Thiamine Deficiency, Thiamine-Responsive Maple Syrup Urine Disease, Thin-Basement-Membrane Nephropathy, Thiolase Deficiency, RCDP, Acyl-CoA Dihydroxyacetonephosphate Acyltransferase, Third and Fourth Pharyngeal Pouch Syndrome, Third Degree Congenital (Complete) Heart Block, Thomsen Disease, Thoracic-Pelvic-Phalangeal Dystrophy, Thoracic Spinal Canal, Thoracoabdominal Syndrome, Thoracoabdominal Ectopia Cordis Syndrome, Three M Syndrome, Three-M Slender-Boned Nanism, Thrombasthenia of Glanzmann and Naegeli. Thrombocythemia Essential, Thrombocytopenia-Absent Radius Syndrome, Thrombocytopenia-Hemangioma Syndrome, Thrombocytopenia-Absent Radii Syndrome, Thrombophilia Hereditary Due to AT III, Thrombotic Thrombocytopenic Purpura, Thromboulcerative Colitis, Thymic Dysplasia with Normal Immunoglobulins, Thymic Agenesis, Thymic Aplasia DiGeorge Type, Thymic Hypoplasia A γ-globulinemias Primary Included, Thymic Hypoplasia DiGeorge Type, Thymus Congenital Aplasia, Tic Douloureux, Tics, Tinel's Syndrome, Tolosa Hunt Syndrome, Tonic Spasmodic Torticollis, Tonic Pupil Syndrome, Tooth and Nail Syndrome, Torch Infection, TORCH Syndrome, Torsion Dystonia, Torticollis, Total Lipodystrophy, Total Anomalous Pulmonary Venous Connection, Touraine's Aphthosis, Tourette Syndrome, Tourette's Disorder, Townes-Brocks Syndrome, Townes Syndrome, Toxic Paralytic Anemia, Toxic Epidermal Necrolysis, Toxopachyosteose Diaphysaire Tibio-Peroniere, Toxopachyosteose, Toxoplasmosis Other Agents Rubella Cytomegalovirus Herpes Simplex, Tracheoesophageal Fistula with or without Esophageal Atresia, Tracheoesophageal Fistula, Transient Neonatal Myasthenia Gravis, Transitional Atrioventricular Septal Defect, Transposition of the Great Arteries, Transtelephonic Monitoring, Transthyretin Methionine-30 Amyloidosis (Type I), Trapezoidocephaly-Multiple Synostosis Syndrome, Treacher Collins Syndrome, Treacher Collins-Franceschetti Syndrome 1, Trevor Disease, Triatrial Heart, Tricho-Dento-Osseous Syndrome, Trichopoliodystrophy, Trichorhinophalangeal Syndrome, Tricuspid atresia, Trifunctional Protein Deficiency, Trigeminal Neuralgia, Triglyceride Storage Disease Impaired Long-Chain Fatty Acid Oxidation, Trigonitis, Trigonocephaly, Trigonocephaly Syndrome, Trigonocephaly “C” Syndrome, Trimethylaminuria, Triphalangeal Thumbs-Hypoplastic Distal Phalanges-Onychodystrophy, Triphalangeal Thumb Syndrome, Triple Symptom Complex of Behcet, Triple X Syndrome, Triplo X Syndrome, Triploid Syndrome, Triploidy, Triploidy Syndrome, Trismus-Pseudocamptodactyly Syndrome, Trisomy, Trisomy G Syndrome, Trisomy X, Trisomy 6q Partial, Trisomy 6q Syndrome Partial, Trisomy 9 Mosaic, Trisomy 9P Syndrome (Partial) Included, Trisomy 11q Partial, Trisomy 14 Mosaic, Trisomy 14 Mosaicism Syndrome, Trisomy 21 Syndrome, Trisomy 22 Mosaic, Trisomy 22 Mosaicism Syndrome, TRPS, TRPS1, TRPS2, TRPS3, True Hermaphroditism, Truncus arteriosus, Tryptophan Malabsorption, Tryptophan Pyrrolase Deficiency, TS, TTP, TTTS, Tuberous Sclerosis, Tubular Ectasia, Turcot Syndrome, Turner Syndrome, Turner-Kieser Syndrome, Turner Phenotype with Normal Chromosomes (Karyotype), Turner-Varny Syndrome, Turricephaly, Twin-Twin Transfusion Syndrome, Twin-to-Twin Transfusion Syndrome, Type A, Type B, Type AB, Type O, Type I Diabetes, Type I Familial Incomplete Male, Type I Familial Incomplete Male Pseudohermaphroditism, Type I Gaucher Disease, Type I (PCCA Deficiency), Type I Tyrosinemia, Type II Gaucher Disease, Type II Histiocytosis, Type II (PCCB Deficiency), Type II Tyrosinnemia, Type IIA Distal Arthrogryposis Multiplex Congenita, Type III Gaucher Disease, Type III Tyrosinemia, Type III Dentinogenesis Imperfecta, Typical Retinoschisis, Tyrosinase Negative Albinism (Type I), Tyrosinase Positive Albinism (Type II), Tyrosinemia Type I Acute Form, Tyrosinemia Type I Chronic Form, Tyrosinosis, UCE, Ulcerative Colitis, Ulcerative Colitis Chronic Non-Specific, Ulnar-Mammary Syndrome, Ulnar-Mammary Syndrome of Pallister, Ulnar Nerve Palsy, UMS, Unclassified FODs, Unconjugated Benign Bilirubinemiav, Underactivity of Parathyroid, Unilateral Ichthyosiform Erythroderma with Ipsilateral Malformations Limb, Unilateral Chondromatosis, Unilateral Defect of Pectoralis Muscle and Syndactyly of the Hand, Unilateral Hemidysplasia Type, Unilateral Megalencephaly, Unilateral Partial Lipodystrophy, Unilateral Renal Agenesis, Unstable Colon, Unverricht Disease, Unverricht-Lundborg Disease, Unverricht-Lundborg-Laf Disease, Unverricht Syndrome, Upper Limb-Cardiovascular Syndrome (Holt-Oram), Upper Motor Neuron Disease, Upper Airway Apnea, Urea Cycle Defects or Disorders, Urea Cycle Disorder Arginase Type, Urea Cycle Disorder Arginino Succinase Type, Urea Cycle Disorders Carbamyl Phosphate Synthetase Type, Urea Cycle Disorder Citrullinemia Type, Urea Cycle Disorders N-Acrtyl Glutamate Synthetase Typ, Urea Cycle Disorder OTC Type, Urethral Syndrome, Urethro-Oculo-Articular Syndrome, Uridine Diphosphate Glucuronosyltransferase Severe Def. Type I, Urinary Tract Defects, Urofacial Syndrome, Uroporphyrinogen III cosynthase, Urticaria pigmentosa, Usher Syndrome, Usher Type I, Usher Type II, Usher Type III, Usher Type IV, Uterine Synechiae, Uoporphyrinogen I-synthase, Uveitis, Uveomeningitis Syndrome, V-CJD, VACTEL Association, VACTERL Association, VACTERL Syndrome, Valgus Calcaneus, Valine Transaminase Deficiency, Valinemia, Valproic Acid, Valproate Acid exposure, Valproic Acid exposure, Valproic acid, Van Buren's Disease, Van der Hoeve-Habertsma-Waardenburg-Gauldi Syndrome, Variable Onset Immunoglobulin Deficiency Dys γ-globulinemia, Variant Creutzfeldt-Jakob Disease (V-CJD), Varicella Embryopathy, Variegate Porphyria, Vascular Birthmarks, Vascular Dementia Binswanger's Type, Vascular Erectile Tumor, Vascular Hemophilia, Vascular Malformations, Vascular Malformations of the Brain, Vasculitis, Vasomotor Ataxia, Vasopressin-Resistant Diabetes Insipidus, Vasopressin-Sensitive Diabetes Insipidus, VATER Association, Vcf Syndrome, Vcfs, Velo Cardio Facial Syndrome, VeloCardioFacial Syndrome, Venereal Arthritis, Venous Malformations, Ventricular Fibrillation, Ventricular Septal Defects, Congenital Ventricular Defects, Ventricular Septal Defect, Ventricular Tachycardia, Venual Malformations, VEOHD, Vermis Aplasia, Vermis Cerebellar Agenesis, Vernal Keratoconjunctivitis, Verruca, Vertebral Anal Tracheoesophageal Esophageal Radial, Vertebral Ankylosing Hyperostosis, Very Early Onset Huntington's Disease, Very Long Chain Acyl-CoA Dehydrogenase (VLCAD) Deficiency, Vestibular Schwannoma, Vestibular Schwannoma Neurofibromatosis, Vestibulocerebellar, Virchow's Oxycephaly, Visceral Xanthogranulomatosis, Visceral Xantho-Granulomatosis, Visceral Myopathy-External Ophthalmoplegia, Visceromegaly-Umbilical Hernia-Macroglossia Syndrome, Visual Amnesia, Vitamin A Deficiency, Vitamin B-1 Deficiency, Vitelline Macular Dystrophy, Vitiligo, Vitiligo Capitis, Vitreoretinal Dystrophy, VKC, VKH Syndrome, VLCAD, Vogt Syndrome, Vogt Cephalosyndactyly, Vogt Koyanagi Harada Syndrome, Von Bechterew-Strumpell Syndrome, Von Eulenburg Paramyotonia Congenita, Von Frey's Syndrome, Von Gierke Disease, Von Hippel-Lindau Syndrome, Von Mikulicz Syndrome, Von Recklinghausen Disease, Von Willebrandt Disease, VP, Vrolik Disease (Type II), VSD, Vulgaris Type Disorder of Cornification, Vulgaris Type Ichthyosis, W Syndrome, Waardenburg Syndrome, Waardenburg-Klein Syndrome, Waardenburg Syndrome Type I (WS1), Waardenburg Syndrome Type II (WS2), Waardenburg Syndrome Type IIA (WS2A), Waardenburg Syndrome Type IIB (WS2B), Waardenburg Syndrome Type III (WS3), Waardenburg Syndrome Type IV (WS4), Waelsch's Syndrome, WAGR Complex, WAGR Syndrome, Waldenstroem's Macroglobulinemia, Waldenstrom's Purpura, Waldenstrom's Syndrome, Waldmann Disease, Walker-Warburg Syndrome, Wandering Spleen, Warburg Syndrome, Warm Antibody Hemolytic Anemia, Warm Reacting Antibody Disease, Wartenberg Syndrome, WAS, Water on the Brain, Watson Syndrome, Watson-Alagille Syndrome, Waterhouse-Friderichsen Syndrome, Waxy Disease, WBS, Weaver Syndrome, Weaver-Smith Syndrome, Weber-Cockayne Disease, Wegener's Granulomatosis, Weil Disease, Weil Syndrome, Weill-Marchesani, Weill-Marchesani Syndrome, Weill-Reyes Syndrome, Weismann-Netter-Stuhl Syndrome, Weissenbacher-Zweymuller Syndrome, Wells Syndrome, Wenckebach, Werdnig-Hoffman Disease, Werdnig-Hoffman Paralysis, Werlhof's Disease, Werner Syndrome, Wernicke's (C) I Syndrome, Wernicke's Aphasia, Wernicke-Korsakoff Syndrome, West Syndrome, Wet Beriberi, WHCR, Whipple's Disease, Whistling Face Syndrome, Whistling Face-Windmill Vane Hand Syndrome, White-Darier Disease, Whitnall-Norman Syndrome, Whorled Nevoid Hypermelanosis, WHS, Wieacker Syndrome, Wieacher Syndrome, Wieacker-Wolff Syndrome, Wiedmann-Beckwith Syndrome, Wiedemann-Rautenstrauch Syndrome, Wildervanck Syndrome, Willebrand-Juergens Disease, Willi-Prader Syndrome, Williams Syndrome, Williams-Beuren Syndrome, Wilms' Tumor, Wilms' Tumor-Aniridia-Gonadoblastoma-Mental Retardation Syndrome, Wilms Tumor Aniridia Gonadoblastoma Mental Retardation, Wilms' Tumor-Aniridia-Genitourinary Anomalies-Mental Retardation Syndrome, Wilms Tumor-Pseudohermaphroditism-Nephropathy, Wilms Tumor and Pseudohermaphroditism, Wilms Tumor-Pseudohermaphroditism-Glomerulopathy, Wilson's Disease, Winchester Syndrome, Winchester-Grossman Syndrome, Wiskott-Aldrich Syndrome, Wiskott-Aldrich Type Immunodeficiency, Witkop Ectodermal Dysplasias, Witkop Tooth-Nail Syndrome, Wittmaack-Ekbom Syndrome, WM Syndrome, WMS, WNS, Wohlfart-Disease, Wohlfart-Kugelberg-Welander Disease, Wolf Syndrome, Wolf-Hirschhorn Chromosome Region (WHCR), Wolf-Hirschhorn Syndrome, Wolff-Parkinson-White Syndrome, Wolfram Syndrome, Wolman Disease (Lysomal Acid Lypase Deficiency), Woody Guthrie's Disease, WPW Syndrome, Writer's Cramp, WS, WSS, WWS, Wyburn-Mason Syndrome, X-Linked Addison's Disease, X-linked Adrenoleukodystrophy (X-ALD), X-linked Adult Onset Spinobulbar Muscular Atrophy, X-linked Adult Spinal Muscular Atrophy, X-Linked A γ-globulinemia with Growth Hormone Deficiency, X-Linked A γ-globulinemia, Lymphoproliferate X-Linked Syndrome, X-linked Cardiomyopathy and Neutropenia, X-Linked Centronuclear Myopathy, X-linked Copper Deficiency, X-linked Copper Malabsorption, X-Linked Dominant Conradi-Hunermann Syndrome, X-Linked Dominant Inheritance Agenesis of Corpus Callosum, X-Linked Dystonia-Parkinsonism, X Linked Ichthyosis, X-Linked Infantile A γ-globulinemia, X-Linked Infantile Nectrotizing Encephalopathy, X-linked Juvenile Retinoschisis, X-linked Lissencephaly, X-linked Lymphoproliferative Syndrome, X-linked Mental Retardation-Clasped Thumb Syndrome, X-Linked Mental Retardation with Hypotonia, X-linked Mental Retardation and Macroorchidism, X-Linked Progressive Combined Variable Immunodeficiency, X-Linked Recessive Conradi-Hunermann Syndrome, X-Linked Recessive Severe Combined Immunodeficiency, X-Linked Retinoschisis, X-linked Spondyloepiphyseal Dysplasia, Xanthine Oxidase Deficiency (Xanthinuria Deficiency, Hereditary), Xanthinuria Deficiency, Hereditary (Xanthine Oxidase Deficiency), Xanthogranulomatosis Generalized, Xanthoma Tuberosum, Xeroderma Pigmentosum, Xeroderma Pigmentosum Dominant Type, Xeroderma Pigmentosum Type A I XPA Classical Form, Xeroderma Pigmentosum Type B II XPB, Xeroderma Pigmentosum Type E V XPE, Xeroderma Pigmentosum Type C III XPC, Xeroderma Pigmentosum Type D IV XPD, Xeroderma Pigmentosum Type F VI XPF, Xeroderma Pigmentosum Type G VII XPG, Xeroderma Pigmentosum Variant Type XP-V, Xeroderma-Talipes- and Enamel Defect, Xerodermic Idiocy, Xerophthalmia, Xerotic Keratitis, XLP, XO Syndrome, XP, XX Male Syndrome, Sex Reversal, XXXXX Syndrome, XXY Syndrome, XYY Syndrome, XYY Chromosome Pattern, Yellow Mutant Albinism, Yellow Nail Syndrome, YKL, Young Female Arteritis, Yunis-Varon Syndrome, YY Syndrome, Z-E Syndrome, Z- and -Protease Inhibitor Deficiency, Zellweger Syndrome, Zellweger Cerebro-Hepato-Renal Syndrome, ZES, Ziehen-Oppenheim Disease (Torsion Dystonia), Zimmermann-Laband Syndrome, Zinc Deficiency Congenital, Zinsser-Cole-Engman Syndrome, ZLS, and/or Zollinger-Ellison Syndrome.

It is to be understood that unless otherwise indicated, the subject invention is not limited to specific formulations of components, manufacturing methods, dosage regimens or the like, as such may vary. It is also to be understood that the terminology used herein is for the purpose of describing particular embodiments only and is not intended to be limiting.

The singular forms “a”, “an” and “the” include plural aspects unless the context clearly dictates otherwise. Thus, for example, reference to a PUFA includes reference to a single PUFA as well as two or more PUFAs or families of PUFAs, an agent includes a single agent, as well as two or more agents.

In describing and claiming the present invention, the following terminology is used in accordance with the definitions set forth below.

The terms “compound”, “active agent”, “chemical agent”, “pharmacologically active agent”, “medicament”, “active” and “drug” are used interchangeably herein to refer to a chemical compound that induces a desired pharmacological and/or physiological effect. All such terms also cover naturally occurring PUFAs and derivatives or modified forms thereof. The terms also encompass pharmaceutically acceptable and pharmacologically active ingredients of those active agents specifically mentioned herein including but not limited to salts, esters, amides, prodrugs, active metabolites, analogs and the like. When the terms “compound”, “active agent”, “chemical agent” “pharmacologically active agent”, “medicament”, “active” and “drug” are used, then it is to be understood that this includes the active agent per se as well as pharmaceutically acceptable, pharmacologically active salts, esters, amides, prodrugs, metabolites, analogs, etc.

Reference to a “compound”, “active agent”, “chemical agent” “pharmacologically active agent”, “medicament”, “active” or “drug” includes combinations of two or more actives such as two or more PUFAs or families of PUFAs. A “combination” also includes multi-part such as a two-part composition where the agents are provided separately and given or dispensed separately or admixed together prior to dispensation. For example, a multi-part pharmaceutical pack may have two or more agents separately maintained.

The term “combination” in addition, encompasses multivalent PUFAs such as two or more PUFAs linked via chemical bond formation.

In addition, the PUFAs may be co-administered with a range of other therapeutic agents including pain relievers such as opiates, preferably morphine, buprenorphine, levomethadone, codeine, tramadol or tilidine, non-sterioidal analgesics, for example, acetylsalicylic acid, paracetamol, diclofenac, meloxicam, ibuprofen, ibuprofen lysinate, ibuprofen in extruded form (as described in WO 99/06038), gabapentine or anti-depressants, preferably imipramine, maprotiline, mianserine, fluoxetine, viloxazine, tranylcypromine and/or moclobemide.

The terms “effective amount” and “therapeutically effective amount” of an agent as used herein mean a sufficient amount of the agent (e.g. an agent such as a PUFA or a derivative thereof) to provide the desired therapeutic or physiological effect or outcome. Undesirable effects, e.g. side effects, are sometimes manifested along with the desired therapeutic effect; hence, a practitioner balances the potential benefits against the potential risks in determining what is an appropriate “effective amount”. The exact amount required will vary from subject to subject, depending on the species, age and general condition of the subject, mode of administration and the like. Thus, it may not be possible to specify an exact “effective amount”. However, an appropriate “effective amount” in any individual case may be determined by one of ordinary skill in the art using only routine experimentation.

By “pharmaceutically acceptable” carrier, excipient or diluent is meant a pharmaceutical vehicle comprised of a material that is not biologically or otherwise undesirable, i.e. the material may be administered to a subject along with the selected active agent without causing any or a substantial adverse reaction. Carriers may include excipients and other additives such as diluents, detergents, coloring agents, wetting or emulsifying agents, pH buffering agents, preservatives, and the like.

Similarly, a “pharmacologically acceptable” salt, ester, emide, prodrug or derivative of a compound as provided herein is a salt, ester, amide, prodrug or derivative that this not biologically or otherwise undesirable.

“Treating” a subject may involve prevention of a condition or other adverse physiological event in a susceptible individual as well as treatment of a clinically symptomatic individual by ameliorating the symptoms of the condition.

A “subject” as used herein refers to an animal, preferably a mammal and more preferably a human who can benefit from the pharmaceutical formulations and methods of the present invention. There is no limitation on the type of animal that could benefit from the presently described pharmaceutical formulations and methods. A subject regardless of whether a human or non-human animal may be referred to as an individual, patient, animal, host or recipient. The compounds and methods of the present invention have applications in human medicine, veterinary medicine as well as in general, domestic or wild animal husbandry. Non-human animals contemplated herein include livestock animals (e.g. sheep, pigs, cows, horses, donkeys), laboratory test animals (e.g. mice, rabbits, rats, guinea pigs), companion animals (e.g. dogs, cats) and captive wild animals.

The term “animals” include avian species such as poultry birds (e.g. chickens, ducks, turkeys, geese) and wild and game birds (e.g. wild ducks, pheasants, emus) and aviary birds.

The present invention is further described by the following non-limiting Examples.

### EXAMPLE 1

### Chemical Engineering of Fats

Compounds were generated by the method described in WO 96/11908, WO 96/13507, WO 97/38688, WO 01/21172 and WO 01/21575 and are designated MP series, PT series and MP-PT hybrids. Molecules of the MP series possess the property of increased stability to oxidative breakdown. This reduced susceptibility to breakdown means that they are far less likely to cause the production of oxygen radicals which is the consequence of the metabolism of the natural omega-3 fatty acids. Molecules of the PT series also have this property but in addition are more soluble. The hybrid MP-PT series possess the above properties and demonstrate an expected outcome of higher antiinflammatory activity.

The structure of a natural fish oil fatty acid, ecosa pentaenoic acid, is shown in structure (a). The features of these types of fatty acids is a long carbon chain, unsaturation (double bonds) and a carboxyl group (acid group) at one end of the chain.

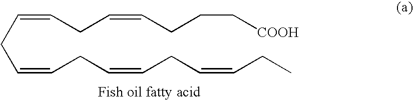

The chemical engineering takes the form of inter alia substituting an oxygen atom (or sulphur) for the carbon, second from the carboxyl group end (b). This is called the β-position. It is this area on the molecule to which the enzyme involved in the metabolism of the fats binds. Due to this change, the enzyme cannot act on this group as efficiently as in the unsubstituted molecule. Thus, the fat is handled differently by body tissues.

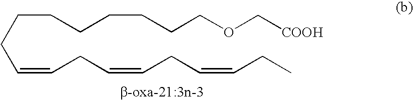

### EXAMPLE 2

### Treating Inflammatory Disease

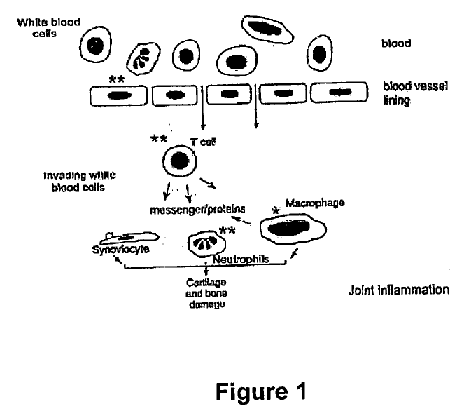

The naturally occurring ω-3 polyunsaturates (such as fish oil) have found use in the treatment of inflammatory diseases. These include the highly debilitating chronic forms such as rheumatoid arthritis, multiple sclerosis, inflammatory bowel disease and systemic lupus erythrocytosis. These are life-long diseases which are managed but cannot be cured. The principle mechanisms involve the T-lymphocyte and macrophage and other white blood cells of the immune system (see FIG. 1). These inappropriately attach to either joint tissue (in arthritis), blood vessel (in lupus), brain (multiple sclerosis) and gut tissue (inflammatory bowel disease) and then damage the tissue.

The PUFAs of the present invention target T-lymphocytes. When T-lymphocytes are exposed to MP5, for example, the cell takes up the fat as a nutritional requirement like any other fat but in this case the MP5 has a slight but vital change in its structure. MP5 stops the flow of a signal inside this cell preventing T-lymphocyte activation.

### EXAMPLE 3

### Transplantation

Management of patients with transplants involves the use of immunosuppressive medications, e.g. cyclosporin which stops T-lymphocyte activation. Rejection of transplanted tissues involves T-lymphocytes and macrophages in a similar manner to the delayed-type hypersensitivity (DTH) reaction. Thus, MP5 has the potential to be used as a suitable immunosuppressive agent in transplantation especially because of the advantages it confers regarding safety compared to presently used immunosuppressants.

### EXAMPLE 4

### Treating Asthma and Allergy

Tissues can be stimulated to produce fatty acid derived hormone like molecules called “eicosanoids” such as the leukotrienes. Production of these in an uncontrolled manner is known to lead to the appearance of serious diseases. These include asthma and allergic conditions. For example, some leukotrienes act on the smooth muscle of the broncus of the airway preventing its relaxation leading to breathing difficulties as in asthma. In accordance with the present invention, a new form of polyunsaturates is provided as inhibitors of eicosanoid production and hence as potential medication to treat asthma and allergic conditions.

### EXAMPLE 5

### Treating Pain

Some evidence has suggested that the novel fats may act on pathways involved in generating pain. As a consequence, some have been screened in two animal models of pain. The engineered polyunsaturates of the present invention were found to act in a similar manner to aspirin but by a different pathway, providing major advantages over toxicity problems associated with long term use of aspirin. One particular useful compound is PT2 (c). This is a polyunsaturated fatty acid which contains an amino acid covalently bound to its carboxyl group:

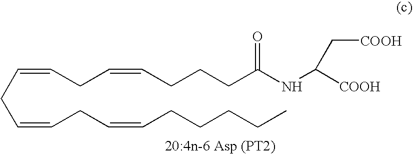

The chemical nature of these novel molecules suggests that they are easily delivered by skin application or oral administration. Investigations have demonstrated that after ingestion, they soon appear in target organs (brain, kidney, lungs or skin). In preliminary studies in rats, active anti-inflamatory levels of these molecules do not display any toxic side effects. The significant anti-inflammatory property as well as the analgesic value of these molecules and their benign non-toxic nature makes the compounds ideal pharmaceuticals.

### EXAMPLE 6

### Analgesic Properties of PT2

### Screening of PT2 on Neutrophil Activation in Vitro

The structure of PT2 is shown in (c) above. In this screening assay, neutrophils were prepared from the blood of healthy volunteers. Freshly collected blood was layered onto a Hypaque-Ficoll medium of density 1.114 and centrifuged at 400 g for 30 mins at room temperature. After centrifugation, the leukocytes resolved into two distinct bands, with neutrophils being present in the second band (Ferrante and Thong, J. Immun. Methods 48:81-85, 1982).

Activation of the neutrophil NADPH oxidase was measured by lucigenin-dependent chemiluminescence following a 10 min incubation of PT2 (20 μM, final concentration) with 1×10⁶ neutrophils from different donors (Power et al, J. Immunol. 159:2952-2959, 1997). Arachidonic acid (20:4, n-6) was used as a positive stimulator of the oxidase

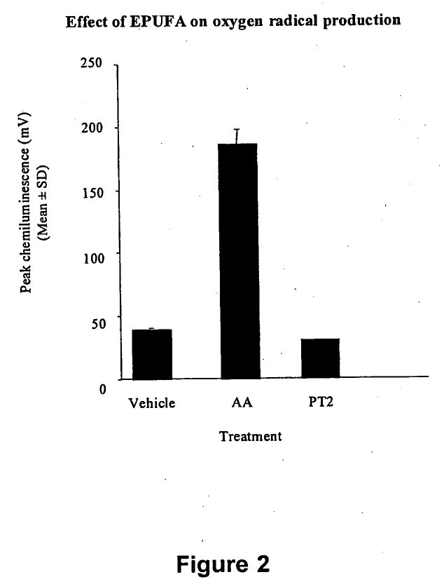

It can be seen that PT2 lacks the ability to stimulate the neutrophil respiratory burst. In contrast, arachidonic acid (and other natural PUFAs) are able to elicit a strong respiratory burst (FIG. 2).

### Analgesic Properties of PT2

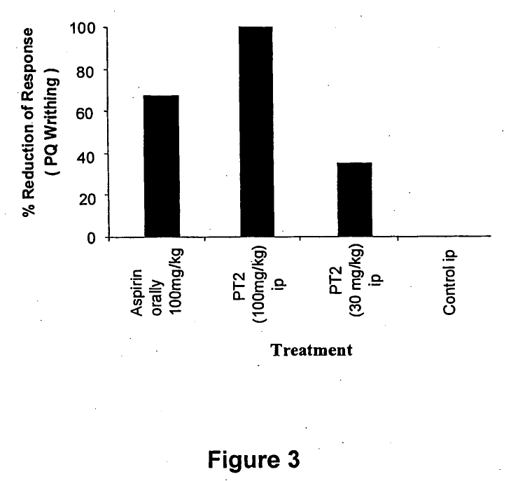

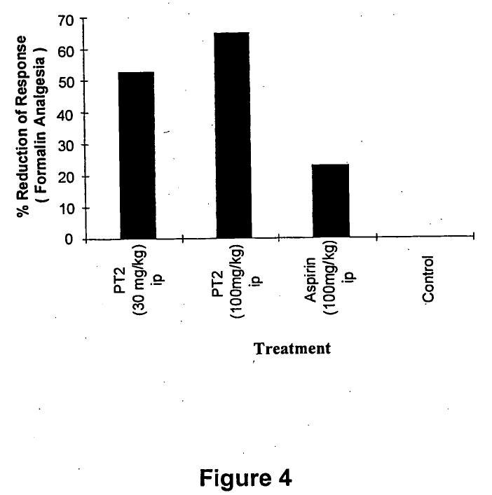

Investigations of the effects of PT2 on pain induced by phenylquinolic acid (PQ writhing) and formalin have been made. In both the PQ writhing test (FIG. 3) and the formalin algesia test (FIG. 4), PT2 administered by intraperitoneal inoculation reduced pain and compared favourably with pain reduction by aspirin (oral, 100 mg/kg). In these tests, the EPUFA was administered 30 min before the pain stimulus and effects recorded over the following 20 min.

Investigations of PT2 in the formalin-induced analgesia model looking specifically at the biphasic response have also been undertaken and are shown in Table 1. It is well documented that in this model, aspirin suppresses only pain related to the inflammatory process (15-20 mins post-administration of formalin), while morphine suppresses pain in both phases of the response (0-5 min and 15-20 min). From Table 1, it can be seen that PT2 acts similarly to aspirin in having its major effect on the later phase of the pain response. MP5 was much less effective in inhibiting pain in this model.

TABLE 1
Effect of PT2 on pain induced by formalin

| | % inhibition of pain response | |
|---|---|---|
| Treatment | Phase I (0-5 mins) | Phase II (15-20 mins) |
| PBS | 0 | 0 |
| PT2 (30 mg/kg) | 0 | 41 |
| PT2 (100 mg/kg) | 30 | 97 |
| MP5 (100 mg/kg) | 34 | 37 |
| Aspirin (300 mg/kg) | 30 | 91 |
| Morphine (10 mg/kg) | 85 | 100 |

Compounds were administered intraperationedly (ip) 30 min prior to the administration of formalin (0.02 ml, 1% solution) via subplantar injection into the right hind paw. Reduction of the induced hind paw licking time recorded during the following 0-5 min period (Phase I response) or 15-20 min period (Phase II response) was determined. The data in Table 1 are the mean responses of 5 animals in each group.

### EXAMPLE 7

### Effects of Nitroanalog (Lx) of PUFA on PKC Activation

The effects of nitroanalogs of PUFAs on PKC activation were determined. Lx compounds at a concentration of 20 μM were incubated with the HL-60 cell line (final condition 10⁶ cells/ml) for 60 min. PKC activation was then attempted to be induced by PMA. PKC enzyme translocation was quantitated by Western blot. The results are shown in Table 2.

TABLE 2
Inhibition of PKC Activation

| PKC | | | | | | | | | |
|---|---|---|---|---|---|---|---|---|---|
| isozyme | Lx1 | Lx2 | Lx3 | Lx4 | Lx5 | Lx6 | Lx7 | Lx8 | Lx9 |
| α | − | − | − | ++ | − | ND | +++ | +++ | − |
| β1 | + | − | − | +++ | − | ND | +++ | ++ | ++ |
| β2 | − | +++ | +++ | +++ | − | ND | +++ | +++ | +++ |
| δ | − | − | − | +++ | − | ND | + | +++ | + |
| ε | − | − | − | − | + | ND | +++ | +++ | + |

+++ = strong inhibition of PKC activation,
− = no inhibition of PKC activation,
ND = not determined

It is evident that there are substantial differences in ability to inhibit the spectrum of five PKC isozymes by the different Lx compounds. For anti-cancer effect, δ and ε are of interest. These have been clearly associated with cell survival (ε) and cell death (δ). In the examples of Lx7 and Lx8, Lx7 kills cancer cells very effectively, yet Lx8 kills cells very poorly. The data in Table 2 show that the activation of apoptotic protective isozyme ε is markedly inhibited by Lx7 without much inhibition of the activation of δ which promotes apoptosis. Therefore, the cell dies. In contrast with Lx8 both isozymes are inhibited. The net effect is survival.

With Lx9, the compound is also strong in killing cancer cells and there is balanced (+) inhibition of both δ and ε.

### EXAMPLE 8

### Treatment of Systemic Vasculature

The aim of the experiment was to establish conditions for optimal activity of β-oxa 23:4n-6 (MP3) in relation to inhibition of up-regulation of adhesion molecular expression on the endothelium in vivo and to determine whether or not MP3 possesses anti-atherosclerotic properties in experimental models.

It is proposed that β-oxa 23:4n-6 (MP3), through its ability to selectively inhibit the IκB kinase-NFκB signalling pathway, inhibits the expression cell adhesion molecules on and the adherence of monocytes to the aortic endothelium, thus preventing the development of atherosclerosis in vivo.

Atherosclerosis is a chronic inflammatory vascular disease which is characterized by a thickening of the vascular wall (atheroma) due to lipid accumulation and infiltration of circulating monocytes and T-lymphocytes. The recruitment of monocytes into the intima in lesion prone-sites is a key event in early atherogenesis. For this to occur, monocytes must first adhere to the endothelium at sites of endothelial injury or dysfunction caused by factors such as oxidized LDL, chylomicron remnants and/or advanced glycation end products (AGE) (Koya et al, Diabetes 47:859-866, 1998). Leukocyte adhesion to the endothelium and the subsequent emigration into the intima is mediated by leukocyte-endothelial cell adhesion molecules (CAMs). These CAMs include the leukocyte L-selectin and the endothelial E-selectin, P-selectin, intercellular adhesion molecule (ICAM)-1 which binds neutrophils and vascular cell adhesion molecule (VCAM)-1 which binds monocytes and T cells. The process begins by E-, L-, and P-selectin-mediated rolling of the leukocytes along the endothelial surface. This is followed by firm adhesion involving the β1 and β2 integrins and immunoglobulin adhesion superfamily members such as ICAM-1 and VCAM-1. The leukocytes then migrate into the intima in response to hypercholesterolemia-induced production of chemoattractants (Koya et al, 1998 supra) and other activating molecules produced by the underlying vascular smooth muscle cells (Chou et al, Curr Biol. 8:1069-77, 1998). The monocytes differentiate into macrophages and ingest modified forms of LDL to become foam cells which give rise to fatty streaks. Activated macrophages release inflammatory cytokines and growth factors that may recruit additional blood monocytes into the developing lesion and stimulate smooth muscle cell migration and proliferation. These processes set the scene for the development of more advanced lesions which include a fibrofatty matrix of connective tissue, smooth muscle and foam cells, followed by the formation of dense fibrous plaques (Koya et al, 1998 supra).

There is overwhelming evidence that CAMs play key roles in atherogenesis. Many atherogenic factors, e.g. hypercholesterolemia, lysophosphatidylcholine and AGE have been reported to increase ICAM-1 and VCAM-1 expression on endothelial cells (Jaken et al, Bioessays 22:245-254, 2000). While oxidized LDL enhances VCAM-1 expression, it only does so in endothelial cells stimulated with cytokines such as tumour necrosis factor α (TNF) and interleukin 1β (Jaken et al, 2000 supra), which are produced at sites of inflammation. In vivo, increases in CAM expression is localized to human arteries with atherosclerotic lesions and in lesion-prone sites on the aortae of mice and rabbits (Koya et al, 1998 supra; Xia et al., J. Clin. Invest. 98:2018-2026, 1996). Studies in animal models have also demonstrated that preventing the expression of CAMs through inactivating mutations caused by homologous recombination (Jaken et al, 2000 supra; Koya et al, J. Clin. Invest. 100:115-126, 1997; Scivittaro et al, Am. J. Physiol. 278:F676-F683, 2000; Way et al, Diabetic Medicine 18:945-959, 2001), and antibody neutralization of CAMs reduce the recruitment of monocytes to atherosclerotic plaques and reduce lesion size (Jaken et al, 2000 supra; Ferrante et al, J. Clin. Invest. 99:1445-1452, 1997). Consequently, strategies to reduce CAM expression are attractive approaches to reduce or impede the development of atherosclerosis and this forms the focus of this application.

One of the essential factors that is required for the up-regulation of CAM expression on the endothelium is the transcription factor, NFκB. The activity of NFκB is tightly regulated by cytokines and other stimuli. In the resting cells, NFκB dimers are sequestered in the cytoplasm by IκB proteins. Upon activation, IκB is phosphorylated by a signalosome complex of IκB kinases. The phosphorylated IκB dissociates from NFκB and undergoes proteosome-mediated degradation, permitting the nuclear translocation of NFκB. Inhibition of NFκB activation results in the suppression of CAM expression. Thus, the NFκB signalling pathway is an attractive target for the development of drugs to suppress inflammatory diseases (Huang et al, Circ. Res. 80:149-158, 1997), including atherosclerosis.

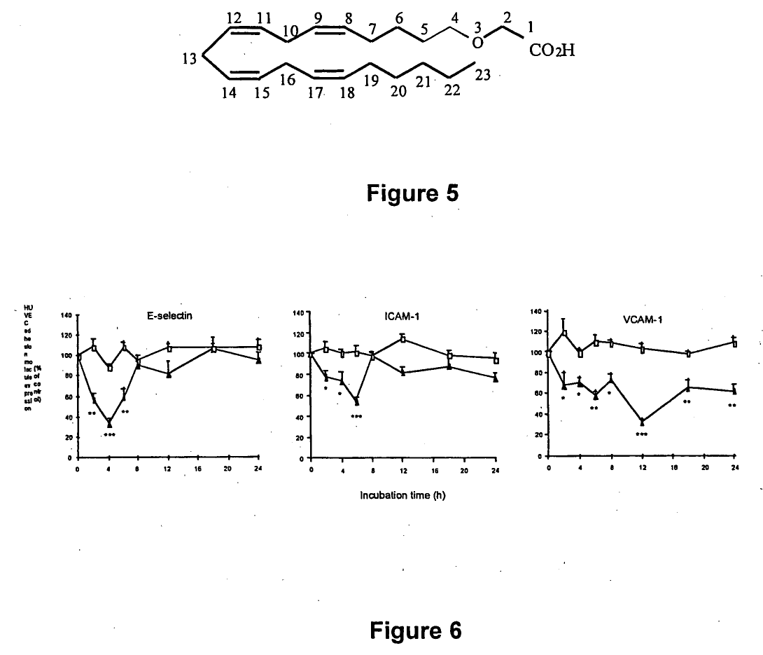

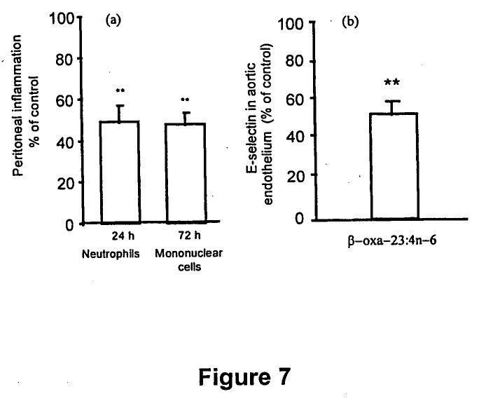

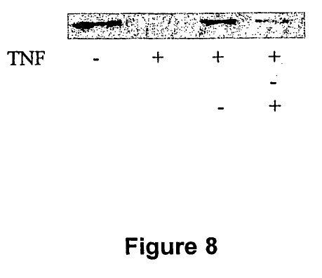

The n-3 fatty acids and fish oil are currently believed to possess cardioprotective actions and one well-studied action is the suppression of CAM expression (Pitt et al, Chem. Phys. Lipids. 92:63-39, 1998). In accordance with the present invention, a novel engineered polyunsaturated fatty acid, β-oxa-23:4n-6 (MP3) (FIG. 5) is identified which has the hall-marks of a new class of pharmaceuticals based on polyunsaturated fatty acids and which can be used to prevent and/or treat cardiovascular disease. MP3 suppresses the expression of CAM and hence leukocyte-endothelium interaction (FIG. 6). This molecule, containing an oxygen atom in the β position of the carbon backbone, is better than docosahexaenoic acid (22:6n-3) at suppressing tumour necrosis factor (TNF)- , lipopolysaccharide (LPS)- or phorbol 12-myristate 13-acetate (PMA)-induced expression of E-selectin, ICAM-1 and VCAM-1 in vitro. However, unlike 22:6n-3 which is a strong stimulator of the phagocyte respiratory burst (AF30) and hence is a promoter of neutrophil-mediated tissue damage, MP3 is relatively poor at stimulating this response. Preliminary studies have found MP3 to be effective in vivo at suppressing LPS-stimulated up-regulation of E-selectin expression in the aortae of mice and prevents the infiltration of leukocytes, including monocytes, into sites of inflammation (FIG. 7). Given at 50 mg/kg intravenously (i.v.), MP3 did not cause any observable signs of distress to the animals for the duration of the experiments (4 days). Preliminary data have also demonstrated that MP3 inhibits the ability of TNF to activate IκB kinase-NFκB signalling pathway (FIG. 5). Docosahexaenoic acid (22:6 n-3) was less effective than MP3 at antagonizing the action of TNF on this pathway, consistent with its weaker ability than MP3 at suppressing CAM expression. The focus of this embodiment of the subject invention is, therefore, the efficacy of MP3 at suppressing adhesion molecule expression in vivo and the development of atherosclerosis.

### EXAMPLE 9

### Animal Models and MP3

The animal model used comprised the apolipoprotein E-deficient (ApoE^{-/-}) mice on a C57BL/6J background. Another model comprised using NZ white rabbits. The ability of MP3 to protect against atherogenesis in two different models, each displaying a different degree of atherosclerosis development, will be a better indicator of MP3's efficacy in protecting against atherogenesis.

ApoE, a 34 kDa glycoprotein that is synthesized predominantly in the liver, is a structural component of all lipoproteins other than LDL. One of its most important functions is to mediate the clearance via the liver of very low density lipoprotein (VLDL) and intermediate density lipoprotein (IDL) via the LDL receptor and of chylomicron remnants via both the LDL receptors and chylomicron remnant receptors (Pitt et al, 1997 supra). Humans with ApoE deficiency have type III hyperlipoproteinemia with elevated plasma cholesterol, early development of atherosclerosis and yellow lipid-laden xanthomatous skin nodules, although triglyceride levels are near normal (Pitt et al, 1997 supra). The ApoE^{-/-} mouse has marked hypercholesterolemia and spontaneously develop lesion patterns characteristics of human atherosclerosis. Extensive fatty streak formation and advanced plaques are observed in many regions of the arterial tree, e.g. aortic root, curvature of the aortic arch, principal branches of the aorta and in the pulmonary and carotid arteries of 30-40 week old ApoE^{-/-} mice (Costabile et al, J. Immunol. 167:2980-2987, 2001; Jirousek et al, J. Med. Chem. 39:2664-2671, 1996). However, signs of early atherosclerotic development is evident in lesion-prone sites, e.g. aortic arch, orifice of the bronchiocephalic artery, and branching sites of the abdominal aorta can be detected as early as 5-6 weeks of age (Dekker et al, Biochem J. 347:285-289, 2000). If fed a Western-type diet, lesion development is accelerated and are more advanced than mice fed a normal chow diet (Costabile et al, J. Immunol. 167:2980-2987; Dekker et al, 2000 supra; Couper et al, Diabetologia 37:533-535, 1994). This mouse is being regarded as an excellent model for histological studies. Of particular relevance to this study is the demonstration in the ApoE^{-/-} mouse that increased expression of CAM at atherosclerosis-prone sites on the aortic endothelium has been observed (Dekker et al, 2000 supra; Couper et al, 1994 supra). More importantly, the concept that blocking CAM expression blocks leukocyte adherence to the endothelium at relevant lesion-prone sites of the aorta and consequently reduces atherogenesis has been validated in the ApoE^{-/-} model, using both genetic approaches and blocking antibodies of various CAM (Koya et al, 1998 supra; Scivittaro et al, 2000 supra, Way et al, 2001 supra, Ferrante et al, 1997 supra). Furthermore, the experiments proposed to be conducted using the ApoE^{-/-} mouse.

The NZ white rabbit develops atherosclerotic lesions when given a high fat-high cholesterol Western-type diet. By 16 weeks, the animals are overtly hypercholesterolemic, and histological studies at this time reveal that 50-80% of aortic intima is covered by atherosclerotic lesions, including fatty streaks and plaques (Kikawa et al, Diabetologia 37:838-841, 1994). Cell proliferation, foam cell and T cell accumulation and lipid deposition are normal in the intima of these animals (Kikawa et al, 1994 supra).

A colony of ApoE^{-/-} mice (Animal Resource Centre, Perth) has been established at the Women's and Children's Hospital, Adelaide, South Australia and in preliminary studies, have confirmed the presence of atherosclerotic lesions in the aortic arch of 16 week old mice fed on standard chow. All ApoE^{-/-} animals for experimentation will be kept on standard chow (4.5% fat, 0.02% cholesterol, w/w) to start with. When appropriate, their diets will be changed to high fat/high cholesterol Western-type diet (w/w) (21% fat—polyunsaturated:saturated=0.07, 0.15% cholesterol).

### EXAMPLE 10

### Effects of MP3 Administration on the Adhesiveness of the Endothelium in Mice

### Adhesion Molecule Expression

It is evident from the data that MP3 inhibits the activation of the IκB-NFκB pathway and the up-regulation of endothelial CAM expression in vitro and LPS-stimulated E-selectin expression in vivo). The aim of this application was to determine whether MP3 also inhibits the expression of VCAM-1 and ICAM-1. For this, C57BL/6J mice (6-8 animals per group, a number which was sufficient in the Balb/c experiments to give statistically significant differences) were pre-treated for 1 day (one dose) or 1 week (one dose/day) with either 40 mg/kg or 80 mg/kg of MP3 intravenously. These concentrations and the route of administration were used previously to demonstrate the suppression of LPS-stimulated E-selectin expression by MP3 in the aorta of Balb/c mice. The fatty acid were presented in DPC (dipalmitoylcholine) micelles (1:4, MP3:DPC, w/w), prepared by sonication. Control mice receive an equivalent amount of DPC. After the pre-treatment period, the mice were injected intraperitoneally with LPS (50 μg), an agent which induces the expression of E-selectin, ICAM-1 and VCAM-1. 24 hr after LPS administration, the animals were sacrificed by CO₂ asphyxiation and the aortae encompassing the ascending part of the aortic arch through to the bifurcation to the common iliac arteries were isolated. Each aorta was then separated into two pieces of equal length and minced. The tissue were fixed in 0.25% v/v glutaraldehyde and processed for enzyme immunoassay. One half of the aorta was stained with monoclonal antibody to mouse VCAM-1 and the other half stained with isotype-matched control IgG. In addition, adhesion molecule expression were assessed by immunohistochemistry using gold-conjugated reagents (Dekker et al, 2000 supra). Once conditions have been optimized with respect to pre-treatment time and the dose of MP3 to be used, the experiments were repeated to examine the effects of MP3 on ICAM-1 expression. As a negative control, MP1 (β-oxa-23:0), a novel fatty acid that is biologically inactive in in vitro assays, was also tested.

Next, the ability of MP3 to reduce the expression of CAM, e.g. VCAM-1, in ApoE^{-/-} mice was investigated. Expression of E-selectin and ICAM-1 was investigated. A previous study found slightly increased expression of VCAM-1 at lesion-prone sites in ApoE^{-/-} mice compared to control mice as early as 5 weeks of age (Dekker et al, 2000 supra). By 8 weeks of age, VCAM-1 staining was more intense and this was further increased in mice fed a Western-type diet. For experiments, the mice were weaned at 4 weeks of age (Dekker et al, 2000 supra). It is proposed to use 12 ApoE^{-/-} mice/group (α=0.5, β=0.1) and these were housed in groups of 6-7 per cage. Some animals have been excluded owing to the presence of severe non-xanthomatous skin lesions or murine urological syndrome (Lallena et al, Mol. Cell. Biol. 19:2180-2188, 1999). At 5 weeks, one group of mice were fed a Western-type diet while the other were maintained on standard chow. The fifth week was chosen to start treatment in order to maximize the difference between control and MP3-treated groups. Two regimes of MP3 treatment were tested. In the first, mice were treated with MP3, DPC or MP1 by intraperitoneal injection a day prior to diet modification. Other studies have demonstrated the engineered fatty acids are effective at suppressing footpad inflammation when administered intraperitoneally (AF45). Treatment continued once daily for 5 or 15 weeks. The mice were sacrificed and adhesion molecule expression were determined as described above. To gauge the degree of suppression of adhesion molecule expression by MP3, the results were compared with those obtained in age-matched ApoE-1- and C57BL/6J mice fed normal chow and treated with DPC. It was expected that chow-fed C57BL/6J mice would have very low levels of CAM expression, chow-fed ApoE^{-/-} mice would have intermediate levels of expression and ApoE^{-/-} mice on Western-type diet would have the highest level of expression. If MP3 is efficacious, the levels of CAM expression would be less than that in DPC- or MP1-treated ApoE^{-/-} mice on Western-type diet. In the second regime, mice were treated with MP3 or MP1, commencing at 8 weeks after diet modification and CAM expression would be determined after 10 weeks of MP3 treatment. This allowed the inventors to determine whether MP3 stopped or reversed atherogenesis.

### Adherence of Macrophages to the Endothelium

To confirm that MP3 reduces the adhesiveness of the endothelium for leukocytes in vivo, an assay based on that described by Ferrante et al (J. Clin. Invest. 99:1445-1452, 1997) would be adopted. Peritoneal macrophages (from C57BL/6J mice) loaded with fluorescent microspheres (Molecular Probes) were injected intravenously into ApoE^{-/-} mice and 48 hr later, the number adhering to the aortic root at the level of the sinus of Valsalva would be scored. Although unprimed blood monocytes would also adhere to the endothelium under identical conditions, the level of adherence was found to be higher with peritoneal macrophages than monocytes and hence peritoneal macrophages were chosen (Ferrante et al, 1997 supra). In ApoE^{-/-} mice, the most advanced lesions were found over the aortic cusps at the level of the sinus of Valsalva (Couper et al, Diabetologia 37:533-535, 1994). Fed on normal chow, increased adherence of monocytes to the endothelium was observable by 6 weeks of age (Couper et al, 1994 supra). Again, 5 week old ApoE^{-/-} mice, in groups of 12 animals, were fed a Western-type diet (optimal period on this diet would be based on the results obtained above). Mice were treated with MP3, MP1 or DPC. On the last day of treatment, mice were injected intravenously; with microsphere-loaded macrophages (1×10⁷ in 0.2 ml). After 48 h, the mice were sacrificed, perfused with heparinized saline by injection through the apex of the left ventricle, and the base of the hearts and ascending aortae isolated, mounted in Tissue Tex freezing media and frozen in liquid N₂. Hematoxylin-stained sections (200 consecutive 5 μm sections) covering the proximal 1 mm of the aortic root were analyzed by light and fluorescent microscopy, and the number of adherent fluorescent monocytes be counted in a blinded fashion. As a positive control, mice which had not been treated with a fatty acid were administered anti-α₄ integrin or ICAM-1 antibody (positive control) prior to the injection of microsphere-loaded macrophages (Ferrante et al, 1997 supra).

To provide another comparison for the degree of suppression of macrophage-endothelium interaction by MP3, macrophage adhesion were also determined in DPC-treated age-matched C57BL/6J mice fed a chow diet. It was envisaged that very few or no macrophages would adhere to the endothelium of these mice.

### EXAMPLE 11

### Effect of MP3 on the Development of Atherosclerosis

The anti-atherosclerotic effect of MP3 were examined first in ApoE^{-/-} mice fed a Western diet. In these mice fed a normal chow diet, foam cell lesions were evident as early as 8 weeks of age and advanced lesions were observable by 15 weeks (Couper et al, 1994 supra). Mice fed a Western diet has more advanced lesions than those on normal chow (Couper et al, 1994 supra).

Mice (12 per group), weaned at 4 weeks of age, were switched from a chow diet to a Western-type diet at 5 weeks of age and maintained on this diet for up to 20 weeks. Daily treatment with MP3 (40 mg/kg), MP1 or DPC commenced at the time of the switch. As a positive control, another group of mice were treated with probucol which suppresses atherogenesis (Suzuma et al, J. Biol. Chem. 277:1047-1057, 2002). At various times, e.g. 5 and 20 weeks after the switch, mice are sacrificed and the degree of atherosclerosis assessed as previously described (Costabile et al, 2001 supra, Jirousek et al, 1996 supra) but with modifications. Briefly, the vascular tree were perfused via the heart with paraformaldehyde (4% w/v) and the heart and aortae down to the bifurcation at the common iliac arteries were isolated intact. The heart and an approximately 5 mm length of ascending aorta were removed from the remainder of the aorta and fixed in formalin. After embedding in paraffin, 4 μm-thick sections at 25 μm intervals were made beginning with the ascending aorta and proceeding through the entire aortic sinus until the ventricular chamber was reached. The sections are stained with Hematoxylin and Eosin and assessed using an Olympus BX51 microscope for foam cell infiltration, cellular proliferation and the presence of fibrous plaques or atheromatous lesions complicated by ulcerations or thrombosis. Images are captured using an Olympus DP12 digital camera. Lesion size (mean cross sectional area) and the percentage of the lumen area occupied by lesion were determined by using a computer assisted image measurement program (Measure Master, Leading Edge, Australia). Where appropriate, sections were stained with elastic Van Gieson and Masson's trichrome to detect collagen. Some sections were also immunostained for macrophages using the anti-mouse macrophage antibody, MAC 3 (Sigma Aldrich). It was also possible to grade these lesions according to the classification described by Stary and co-workers (Lallena et al, Mol. Cell. Biol. 19:2180-2188, 1999). The remaining section of the aorta were pinned on to a board, sectioned longitudinally, one half fixed in formalin, stained with Oil red O/Sudan IV and counter-stained with Hematoxylin Eosin to detect lipid laden cells. The other half were fixed and 12 μm frozen sections in the abdominal aorta around the renal arteries were stained to detect the lipid laden cells. Lesion size were determined as described above and results expressed as the percentage of lesion area relative to the total internal surface. Older mice (30 weeks) known to have advanced lesions were also treated with MP3 over a period of 15 weeks to determine whether atherosclerosis could be halted or reversed.

The experiments above were then repeated but with MP3 or control agents given orally. Being a fatty acid, it was expected for MP3 to be absorbed across the intestinal wall into the blood stream. Indeed, previous studies with another engineered fatty acid, MP5, have demonstrated that this fatty acid is present in the blood and various organs after oral administration. Studies in dogs have shown that a saturated β-oxa fatty acid is readily absorbed when given orally (Hii et al, J. Biol. Chem. 266:20238-20243, 1991). Thus, it was investigated whether MP3 is efficacious at suppressing atherosclerosis when given orally. The experiment essentially followed the schedule outlined above to determine whether MP3 prevented the development of atherosclerosis. Mice were administered MP3 or a control compound daily by gavage for the appropriate length of time (see above) and the degree of atherosclerosis assessed. Finally, the anti-atherosclerotic effects of MP3 were tested using NZ white rabbits. After 16 weeks on a high cholesterol diet, these animals were shown to be overtly hypercholesterolemic and histological studies at this time show that 50-80% of aortic intima was covered by atherosclerotic lesions, including fatty streaks and plaques. For the experiments, rabbits fed on standard chow, weighing 1.8-2.2 kg and with serum cholesterol of less than 100 mg/dl, were selected. They were divided into five groups of eight animals each: standard chow+DPC, standard chow+MP3, high cholesterol (2% w/w) diet+DPC, high cholesterol diet+MP3 and high cholesterol diet+probucol (0.25%). Treatment with MP3 (40 mg/kg) would coincide with the switch to a high cholesterol diet. The animals were kept on their diets and treated with MP3 for 16 weeks. At the end of this period, the animals were sacrificed by heart puncture under ketamine. The thoracic aortae were removed, sectioned longitudinally, one half pinned on to boards, fixed and stained with Oil red O. The sections were photographed as described above and the extent of Oil red O positive area between the first and fifth intercostal aortic branches were determined in a blinded fashion and expressed as a percentage of the total internal surface. The other half were processed for light microscopy and 4 μm sections were taken from a 5 mm segment around the first intercostal branch. After mounting on slides, lesion area was assessed as described above. These sections were also immunostained for macrophages using the rabbit macrophage antibody, RAM 11 (Dako, Calif.).

Statistical analysis of the results were performed by one way ANOVA followed by an appropriate post test, e.g. Bonferroni test for multiple comparison or Mann-Whitney U-test. Results were considered statistically significant when P<0.05.

### EXAMPLE 12

### Effects of PUFAs on Diabetes

The overall aim of this Example was to evaluate the potential for a chemically engineered polyunsaturated fatty acid, MP5 (β-oxa-21:3n-3), to treat pathogenesis associated with diabetes by targeting the protein kinase C (PKC) system. The specific aims were to:
(1) characterize the effects of MP5 on glucose or advanced glycosylation end product-stimulated activation of PKC, e.g. prevent agonist-stimulated association of PKCβ with a particulate fraction in mesangial cells;
(2) determine whether esterification of MP5 into diacylglycerol was essential for the action of MP5;
(3) investigate whether MP5 is efficacious at preventing glucose-induced responses in vitro, e.g. glucose-stimulated TGFβ production in mesangial cells, and in vivo in streptozotocin diabetic rats.

MP5, by virtue of its incorporation into membrane phospholipids and diacylglycerol, selectively targets the protein kinase Cβ isoforms in mesangial cells by preventing PKC translocation. This prevents glucose and advanced glycosylation end products from causing functional changes in mesangial cells in culture and in the kidneys of streptozotocin diabetic rats.

The majority of diabetic patients were not able to attain near normal glycaemia. This pre-disposed them to the development of diabetic microvascular and macrovascular complications. Therefore, novel approaches to prevent the effects of hyperglycemia were essential to the future management of diabetes. Recent focus centred on identifying the hyperglycemia-induced biochemical changes that were significant in causing vascular and neurological dysfunction. One consistent observation was that glucose stimulated the activity and expression of protein kinase C (PKC) in tissues at risk of developing diabetic complications (Koya et al., 1998 supra). This raises the likelihood that PKC may be an important mediator of the actions of glucose in these tissues.

PKC is a ubiquitous phospholipid-activated Ser/Thr kinase. Consisting of at least 11 isozymes, PKC is divided into classical (α, βI, βII and γ), novel (δ, ε, θ, η, and \tilde{μ}PKD) and atypical (ζ and ι/λ) isozymes (Chou et al, 1998 supra). The activity of the classical PKC isozymes is stimulated when diacylglycerol (DAG) and Ca²⁺ accumulate in appropriately stimulated cells while activation of the novel PKC isozymes requires only DAG. Both the classical and novel PKC can also be activated directly by phorbol esters such as phorbol 12-myristate 13-acetate (PMA). Activation of the atypical isozymes is regulated by other means, e.g. ceramide and phosphorylation (Chou et al, 1998 supra). In unstimulated cells, PKC is generally found in the cytoplasm and it translocates to particulate fractions upon stimulation where it associates with binding partners such as RACKs (receptor for activated C kinase) (Jaken et al, 2000 supra).

PKC regulates a diverse range of cellular processes in an isozyme(s)-specific manner. There is very strong evidence to implicate PKC, especially PKCβ, in mediating the actions of glucose in diabetes. This includes the activation of PKCβ in renal glomeruli, retina, aorta and heart of diabetic animals and in glucose-stimulated cells (Koya et al., 1998 supra). More importantly, inhibition of PKCβ with the PKCβ-specific inhibitor, LY333531, reverses/blocks the actions of glucose in these tissues. For example, in the retinae of diabetic patients and animals with a short history of the disease, retinal blood flow is decreased due to glucose-induced vasoconstriction (Koya et al., 1998 supra). While direct stimulation of PKC with a phorbol ester causes retinal vasoconstriction, inhibition of PKC activity normalizes retinal blood flow in diabetic dogs (Koya et al., 1998 supra). Hyperglycemia-induced increase in endothelial cell permeability to macromolecules, another characteristic systemic vascular abnormality in diabetes, can be reproduced by the addition PMA and PKCβ has been implicated in causing this change in permeability (Koya et al., 1998 supra). In the microvessels and macrovessels, hyperglycemia-induced vasodilation and increase in contractility, respectively, can be reversed by inhibitors of PKC (Koya et al., 1998 supra). One of the factors that cause these changes in renal tissues is the glucose-induced increase in the activity of the renin-angiotensin system, and PKC has been implicated in the actions of angiotensin (Koya et al., 1998 supra). Increased angiogenesis, neovascularization and over-expression of the extracellular matrix proteins are also hallmarks of diabetes, and these are believed to be due to glucose-induced production of vascular-endothelial cell growth factor (VEGF) and TGFβ. While inhibition of PKC inhibits the actions of VEGF on endothelial cell proliferation (Xia et al., J. Clin. Invest. 98:2018-2026, 1996), inhibition of PKCβ effectively blocks hyperglycemia-induced production of TGFβ in mesangial cells and renal glomeruli (Koya et al, 1997 supra) and the associated expansion of the extracellular matrix. Furthermore, decreases in the activity of the Na⁺—K⁺-ATPase in vascular and neuronal tissues are widely reported in diabetic patients, and glucose-induced reduction in the Na⁺—K⁺ ATPase activity in mesangial cells and aortic smooth muscle cells has been found to be dependent on PKCβ. Current evidence also suggests that arachidonic acid, produced by the sequential activation of PKC and cytosolic phospholipase A₂, is responsible for the action of glucose on the sodium pump.

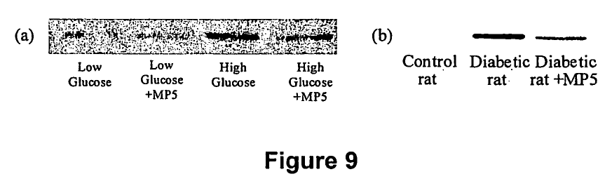

When the engineered polyunsaturates were examined for biological activity in human umbilical vein endothelial cells (HUVEC) and other cell-types, several were found to display a more selective range of actions than their natural counterparts. One of these, MP5 (β-oxa-21:3n-3), inhibited PMA-stimulated translocation of PKCβI and βII to the particulate fraction in these cell-types. MP5 had minimal effects (<15%) on PKCε translocation and no effects on the translocation of the other PKC isozymes in Jurkat cells. Preliminary data from glucose-stimulated mesangial cells (FIG. 9a) and the glomeruli of diabetic rats (FIG. 9b) have confirmed the ability of MP5 to prevent the translocation of PKCβI, the main β isoform in mesangial cells, to a particulate fraction. Long term in vivo experiments (up to three months) showed that treatment with MP5 (up to 100 mg/kg) had no visible adverse effects on the well-being of the animals, e.g. coat appearance and activity/mobility, and exerted no adverse effects on liver and kidney function and electrolyte levels. These data indicate that MP5 has the hallmarks of a lead compound for blocking the actions of glucose.

While LY33531 inhibits PKCβ by binding to the ATP-binding site of the kinase (Jirousek et al, 1996 supra), MP5 acts by reducing the association of PKCβ with the particulate fraction. Because of this unique mode of action, i.e. MP5 is not a kinase inhibitor, the likelihood of MP5 directly affecting the activity of any kinase is extremely remote. The potential for a kinase inhibitor such as LY333531 and derivatives to affect the activity of other kinases has been recently voiced (Jirousek et al, 1996 supra). Depending on the concentrations used, the preliminary data in HUVEC have demonstrated that MP5 is able to distinguish between PKCβI and PKCβII.

The objective of this Example was to determine whether EPUFA such as MP5 could be developed into novel therapeutics to prevent the severe and life-threatening pathology associated with diabetes using the kidney as a model. This is achieved by testing the ability of MP5 to block glucose- or AGE-stimulated activation of PKCβ and whether this requires esterification of MP5 into membrane phospholipids. Finally, MP5 is tested for efficacious at inhibiting glucose-stimulated responses in mesangial cells in vitro and hyperglycemia-induced renal damage in an experimental animal model of diabetes.

### EXAMPLE 13

### Effects of MP5 (β-oxa 21:3n-3) on the Activation/Translocation of Different PKC Isozymes

Since glucose has been demonstrated to stimulate the translocation of PKCα and βI in mesangial cells (Koya et al, 1997 supra), the effects of MP5 on the association of PKCα and βI with the particulate fraction in glucose-stimulated mesangial cells is determined. Preliminary studies in glucose-stimulated mesangial cells have indicated that MP5 can prevent PKCβI from translocating the particulate fraction (FIG. 9). Mesangial cells are prepared as previously described (Couper et al, 1994 supra).

Four groups of cells were set up: 5.5 mM glucose+ethanol (0.1% w/v or v/v), 5.5 mM glucose+MP5 (20 μM which is effective in the studies), 25 mM glucose+ethanol and 25 mM glucose+MP5. In some experiments, an additional group of cells are treated with MP1 (β-oxa 23:0) (20 μM), an inactive fatty acid. Cells were pre-incubated (30 min-24 h) with MP5 before being challenged with glucose for 4 days. The kinetics studies have found that glucose-stimulated PKC translocation in mesangial cells reaches a maximum at day 4 of treatment, consistent with findings from previous reports (Koya et al 1997 supra). Cells are then be washed, sonicated and the amount of each PKC isozyme in the particulate fraction determined by Western blot analysis. The soluble fractions were kept for estimation of soluble PKC (non-particulate fraction-associated). The inventors' studies demonstrated that a 30 min pre-treatment period with MP5 was sufficient to inhibit agonist-stimulated increase in the association of PKC with the particulate fraction and block agonist-stimulated functional responses, but the IC₅₀ decreased with increasing time of pre-treatment. The cells remained viable throughout the period of study as judged by the trypan blue exclusion test. The studies were repeated with cells stimulated with AGE-human serum albumin (HSA) since AGE-HSA was demonstrated to stimulate PKCβII translocation without affecting PKCα translocation (Scivittaro et al, 2000 supra). AGE-HSA (pyrogen-free) were prepared by incubating the protein in the presence of glucose essentially as previously described (Scivittaro et al, 2000 supra). Control HSA were incubated in the absence of glucose. Analysis of the extent of AGE-HSA formation and AGE-HSA purification were carried out as described (Scivittaro et al, 2000 supra). After removal of remaining glucose (centricon, 10 kDa cut-off), AGE-HSA were tested at 0.1-10 μM.

The effects of MP5 on glucose-stimulated PKC expression were investigated since glucose increased the expression of PKCβ in mesangial cells (Koya et al, 1997 supra). This was achieved by determining the level of PKCβ mRNA by slot blot analysis (Ferrante et al, 1997 supra). All classical and novel PKC isozymes were probed. The level of PKC mRNA are normalized by comparison with the level of glyceraldehyde 3-phosphate dehydrogenase mRNA in the same sample.

Mesangial cells express PKCα, βI, ε, δ and ζ (Koya et al, 1997 supra, Kikkawa et al, 1994 supra), and βII at lower levels (Koya et al, 1997 supra). To examine the effect of MP5 on the ability of other PKC isozymes in mesangial cells to translocate to the particulate fraction, the cells were pre-treated with MP5 before being stimulated with PMA (1-100 nM). This agent stimulated the translocation of all the classical and novel isozymes. The studies in HUVEC have demonstrated that MP5 suppressed PMA-stimulated association of PKCβI and βII with the particulate fraction. The studies were extended to other isozymes. For PKCγ (expressed mainly by neuronal cells), the effect of MP5 are tested using PMA-stimulated PC12 rat pheochromocytoma cells. To assess the effect of MP5 on the activation of atypical PKC isozymes such as PKCζ NIH3T3 cells were pre-treated with MP5 before being stimulated with tumour necrosis factor [symbol] (1000 U/ml) which stimulates PKCζ activity in these cells (Lallena et al, 1999 supra). The isozyme were immunoprecipitated (antibody from Santa Cruz) and kinase activity was determined using a PKCε pseudosubstrate peptide or myelin basic protein as a substrate (Suzuma et al, 2002 supra).

### EXAMPLE 14

### Incorporation of MP5 into Diacylglycerol

In MP5-treated HUVEC, the ratio of non-esterified MP5 to non-esterified arachidonic acid is as high as 5:1. Thus, incubation of mesangial cells with glucose and MP5 is likely to result in the formation of DAG that contains MP5. The formation of a more polar and conformationally different MP5-containing DAG can conceivably interfere with the ability of natural DAG to stimulate PKC translocation. The hypothesis is first tested that MP5 does not inhibit glucose-stimulated accumulation of DAG but leads to the formation of M5-containing DAG. Mesangial cells were incubated with MP5 (30 μM, 1 h) before increasing the glucose concentration to 25 mM. After 4 days, lipids were extracted (CHCl₃:CH₃OH=2:1), DAG isolated by thin layer chromatography (TLC), eluted from the silica and the presence of MP5 in DAG were determined and quantitated by GC/GCMS after hydrolysis of the esterified fatty acids (Hii et al, 1991 supra) from DAG and conversion of the liberated MP5 to its methyl ester derivative. 19:0 methyl ester were used as a standard (Robinson et al, Biochem J. 336:611-617, 1998) and this method is used successfully to determine the content of EPUFA in DAG and phospholipids. To quantitate DAG, an assay developed and validated in mesangial cells were used (Musial et al, J. Biol. Chem. 270:21632-21638, 1995). DAG that is extracted from the TLC plates were acetylated with ¹⁴C-acetic anhydride and pyridine. After rechromatography by TLC, the DAG-acetate, after autoradiography, were subjected to liquid scintillation counting. Some cultures were incubated with the inactive MP1. If MP1 was also incorporated, it would imply that the biological activity of an EPUFA was governed by its structural elements. The rationale for the synthesis of EPUFA was based on this concept. The esterification of MP5 into DAG was next determined as to whether this was required for the inhibition of glucose-stimulated PKCβI-particulate fraction association. It is mandatory that fatty acids were converted to their coenzyme A derivatives for metabolism, including esterification into DAG. Cells were pre-incubated with DMSO (control) or triacin C, a commonly used inhibitor of long chain acyl coenzyme A synthetase (Korchak et al, J. Biol. Chem. 269:30281-30287, 1994), for 10 min before being incubated with MP5 (20 μM) for 1 hr since this pre-treatment time was sufficient to block PMA-stimulated PKCβ translocation in glucose stimulated HUTVEC and mesangial cells. The cells were then stimulated with PMA (100 nM, 0.5-3 min) or vehicle (DMSO) instead of glucose to shorten the time required for PKC activation and to minimize the effect of triacin C on normal fatty acid metabolism. The amount of PKCβI and βII in particulate fraction were determined. Triacin C-mediated inhibition of ³H-palmitic acid incorporation into DAG and phosphatidylcholine (PC) (Hii et al, 1991 supra) serves to confirm that the triacin C is active.

### EXAMPLE 15

### Effects of MP5 on Glucose- or Diabetes-Induced Functional Changes Associated with Pathogenesis

Once it was confirmed that MP5 inhibited the association of PKCβ with the particulate fraction in mesangial cells, MP5 were examined for its ability to inhibit in vitro parameters of glucose-induced functional changes. The data were normalised for cellular protein content. These investigations were followed by an examination of the efficacy of MP5 in protecting against hyperglycemia-induced functional changes in the kidneys of diabetic rats.

### In Vitro Studies

### Suppression of Glucose-Stimulated Production of TGFβ

Glucose-induced expansion of the extracellular matrix via the production of TGFβ by mesangial cells was a major contributor to diabetic nephropathy and this action of glucose could be blocked by inhibitors of PKCβ (Koya et al, 1998 supra, Koya et al, 1997 supra). Thus, the ability of MP5 to inhibit glucose-stimulated TGFβ production were investigated. Cells were pre-treated with MP5 (see above) in DMEM (5.5 mM glucose) before being stimulated with glucose (25 mM) for 4 days. The amount of TGFβ present in the incubation medium was determined using a commercially available ELISA kit (Biosource, USA). MP1 was used as a negative control. LY333531 was tested as a positive control.

### Suppression of Phospholipase A₂ Activity

Hyperglycemia-induced production of prostaglandin E₂ and I₂ have been implicated as contributing factors to glomerular hyperfiltration in diabetes (Koya et al, 1998 supra). These vasodilatory prostanoids were derived from arachidonic acid via the action of the cytosolic phospholipase A₂ (cPLA₂), and glucose stimulates the activity of cPLA₂ in mesangial cells in a PKC-dependent manner (Koya et al, 1997 supra). Cells were pre-treated with MP5 before being stimulated with glucose. At the end of the incubation period, the cells were harvested, lysed and the activity of cPLA₂ determined (Robinson et al, 1998 supra) using a commercial kit (Cayman Chemical, USA). The ability of MP5 to inhibit PKC-independent activation of cPLA₂ by ionomycin (0.1 μM, 15 min) was also determined. If the fatty acid acted by specifically inhibiting PKCβ translocation, ionomycin-stimulated cPLA₂ activity would not be affected by the fatty acid.

### Na⁺K⁺ ATPase:

In addition to its central role in nerve function, the Na⁺—K⁺ ATPase may also regulate barrier permeability and cellular integrity in the glomeruli. Glucose and diabetes have been widely reported to inhibit the activity of the Na⁺—K⁺-ATPase in the glomeruli/mesangial cells and aortic smooth muscle cells (Koya et al, 1998 supra, Koya et al, 1997 supra). This effect was believed to be due to glucose-stimulated accumulation of arachidonic acid, and inhibition of PKCβ prevented the inhibition of the Na⁺—K⁺ ATPase by glucose in aortic smooth muscle cells and mesangial cells (Koya et al, 1998 supra, Koya et al, 1997 supra). To determine whether MP5 blocked the action of glucose on the activity of the Na⁺—K⁺ ATPase, the cells were pre-incubated with MP5 (see above) and then incubated in the presence of 25 mM glucose for 4 days. ⁸⁶Rb⁺ uptake, a standard assay for Na⁺—K⁺ ATPase, was determined as described (Koya et al, 1997 supra).

### In Vivo Studies

The ability of MP5 to inhibit renal TGFβ production and albuminuria in streptozotocin-induced diabetic Sprague-Dawley rats were investigated. MP5 is non toxic to rats given at up to 100 mg/kg chronically as determined by liver and kidney biochemical and electrolyte markers. It is taken up by tissues, including kidneys, and incorporated into phospholipids following oral administration. Animals (130-150 g) were placed randomly in one of five groups: control, MP5-treated, diabetic, diabetic+MP5 and diabetic+MP1. Power analysis (α=0.5, β=0.1) (expecting at least a 50% reduction and an SD of 30% of mean) indicate that 7-8 animals/group were needed. The rats were rendered diabetic using streptozotocin (65 mg/kg. I.P) and blood glucose levels were measured 48 hr later to confirm the onset of diabetes (glucose>15 mM). Control and diabetic groups were administered vehicle (ethanol) or an EPUFA by gavage. Two doses were tested, 20 mg/kg and 50 mg/kg. The studies on the actions of EPUFA in vivo have demonstrated that the fatty acids were effective when given orally. Treatment was once daily for a period of 12 weeks (Koya et al, 1997 supra). Blood glucose was measured every week. The animals were treated with 2 U of long acting insulin daily to maintain body weight and to prevent ketoacidosis but without normalizing hyperglycemia. At the end of the MP5 treatment period, rats were sacrificed and the level of TGFβ mRNA (Kikkawa et al 1994 supra) in the glomeruli were determined by slot blot analysis (Ferrante et al, 1997 supra) and normalized by comparison with the level of GAPDH (glyceraldehyde 3-phosphate dehydrogenase) mRNA in the same sample (Ferrante et al, 1997 supra). The amount of albumin in the urine were measured by using a commercial kit (EXOCELL Inc. Philadelphia, Pa.). It was determined whether MP5 could halt/reverse complications in animals with more advanced (e.g. 15 weeks) diabetes. The diabetic animals were treated with MP5 (20 or 50 mg/kg, depending on the above results) once daily together with insulin (as above) for 12 weeks. Other parameters such as the production of hepatocyte growth factor by the glomeruli (Couper et al, 1994 supra) may also be examined if time permits.

### EXAMPLE 16

### Polyunsaturated Fatty Acid (PUFA) Regulation of the Activation of the IκB Pathway

The objective of this Example is to make agents which are PUFA based, with many of the properties of PUFA, such as absorption following oral administration and incorporation into membrane phospholipids, but with more selective biological activities skewed towards anti-inflammatory effects.

### Materials and Methods

### Fatty Acids

The fatty acids, arachidonic acid (20:4n-6), ecosa pentaenoic acid 20:5n-3(EPA) and docosahexaenoic acid 22:6n-3(DHA) were purchased from Sigma Chemical Co, St Louis. Mo. The β-oxa and β-thia compounds were synthesized using published techniques. β-oxa-23:4n-6 methyl ester was formed by the treatment of β-oxa-23:4n-6 with diazomethane in diethyl ether, β-oxa-23:0 was prepared by hydrogenation of β-oxa-23:4n-6 in the presence of platinum oxide (Huang et al, 1997 supra), and 18-monohydroperoxy-β-oxa-23:4n-6 was prepared by incubation of β-oxa-23:4n-6 with soybean lipoxidase (Huang et al, 1997 supra). 18-monohydroxy-β-oxa-23:4n-6 was obtained by reduction of the 18-monohydroperoxy product with sodium borohydride (Huang et al, 1997 supra).

The products were not individually purified, but were separated by ID TLC (Et2O/hexane/acetic acid; 60:40:1). The appropriate lipid zones were visualized under UV light with dichlorofluoroscein (0.2% v/v) in ethanol and identified by comparison of RFs with those of similar structured analogs. No other mono-hydroxylated materials were evident, but more polar polyhydroxylated compounds would have been present in the polar fractions of the chromatogram (at the baseline).

Fatty acids and derivatives were dissolved in ethanol (0.1% final, v/v) (in vitro), dipalmitoylphosphatidylcholine (DPC) (Ferrante et al, 1997 supra) or DSMO (7% v/v) (in vivo). At these concentrations these diluents did not affect cellular functions. Thin-layer chromatography and gas-liquid chromatography-mass spectrometry showed that the lipids were at least 98% pure.

### Neutrophil Respiratory Burst

Neutrophil respiratory burst was determined as previously described (Li et al, J. Clin. Invest. 97:1605-1609, 1996).

### Neutrophil Adhesion to Human Umbilical Vein Endothelial Cells (HUVEC)

Adhesion of neutrophils, prepared by the rapid-single-step method (Ferrante et al, J. Immun. Methods 36:109-117, 1980), to HUVEC isolated from umbilical cords was carried out essentially as described (Huang et al, 1997 supra).

### Measurement of Endothelial Cell Adhesion Molecules

HUVEC were stimulated with TNF, bacterial lipopolysaccharide (LPS) or PMA for 24 hr. Expression of E-selectin, ICAM-1 and VCAM-1 was determined by an enzyme-linked immunosorbent assay (ELISA) or as mRNA using a slot blot technique (Huang et al 1997 supra).

The LPS-induced expression of E-selectin in the aortic endothelium of BALB/c mice was determined following injection of 50 μg LPS intraperitoneally and aortas encompassing the ascending part of the aortic arch through to the bifurcation to the common iliac arteries isolated after 5 hr. Each was cut into two pieces of equal length, minced, fixed in 0.25% v/v glutaraldehyde, incubated with a monoclonal antibody to mouse E-selectin (one half) or isotype matched control (other half) (Becton Dickinson, Ca) followed by an HRP-conjugated secondary antibody and then with the substrate ABTS (ELISA method).

### Measurements of Lipoxygenase Products

Lipids were extracted from the HUVEC culture medium and oxygenated fatty acid derivatives were isolated by thin-layer chromatography. The recovered oxygenated derivatives of β-oxa-23:4n-6 were characterized by electrospray mass spectrometry according to Pitt et al (Pitt et al, 1998 supra). Electrospray ionisation mass spectra (ESI-MS) were recorded on a Finnigan LCQ spectrometer, operating at a spray voltage of 5.20 kV, capillary temperature of 200° C. and capillary voltage of 35V. Analyses were performed in methanol, and ions were reported as their M+H+, M+Na+, or M+K+ ions.

### Preparation of Cell Lysates

Cell lysates were prepared as previously described for IκB kinase (IKK) activity (Lee et al, Proc. Natl. Acad. Sci. USA 95:9319-9324, 1998), IκBα degradation, MAP kinase activity (Hii et al, 1998 supra) and nuclear translocation of NFκB (p65 rel) (Jersmann et al, Infect. Immun. 69:1273-1279, 2001).

### Western Blot Analysis to Detect NFκB and IκBα

Proteins (50 μg) were separated by SDS PAGE (12% w/v gel), transferred to nitrocellulose and probed with an anti-NFκB p65 or anti-IκBα antibody (Santa Cruz Biotech, Santa Cruz, Calif.). Immunocomplexes were detected by enhanced chemiluminescence (Hii et al, 1998 supra).

### IκB Kinase Kinase (IKK) Assay

IKK was immunoprecipitated with anti-IKKα (M-280) antibody (sc-7182, Santa Cruz, Biotech) and kinase activity determined using GST-IκBA (residues 5-55) as previously described (Lee et al, 1998 supra). Proteins were fractionated by SDS PAGE and radioactivity associated with GST-IκBα (residues 5-55) determined using an instant imager.

Measurement of the Activity of p38, ERK and JNK

ERK and p38 were precipitated with anti-ERK2 (C-14, sc-154) and anti-p38 (C-20, sc-535) antibody, respectively (Santa Cruz Biotech) and the activity determined using myelin basic protein as a substrate (Hii et al, 1995 supra, Hii et al, 1998 supra). JNK activity was determined in a solid phase assay using GST c-Jun (residues-1-79) as a substrate (Hii et al, 1995 supra, Hii et al, 1998 supra).

### Inflammatory Reactions

Effect of MP3 (β-oxa-23:4n-6) on the in vivo inflammatory response was measured as a delayed type hypersensitivity (DTH) reaction and LPS-induced influx of neutrophils and mononuclear cells in the peritoneal cavity in BALB/c mice. For DTH experiments, mice were injected subcutaneously with 100 μl of 10% hematocrit sheep erythrocytes, challenged with the antigen (25 μl of 40%) in the hind foot pad 6 days later and the degree of foot pad swelling measured 48 hr later (Ferrante et al, Clin. & Exp. Immunol. 38:70-76, 1979). One hour prior to challenge mice were given 10 mg/kg body weight of the fatty acid, intraperitoneally. For peritoneal inflammation, mice were injected with 50 μg of LPS intraperitoneally 6 hr after intravenous fatty acid treatment. At 24 hr and 72 hr the peritoneal exudate was harvested and the number and proportion of neutrophils and macrophages determined microscopically from Giemsa stained smears.

### Results

### Effects on the Neutrophil Respiratory Burst

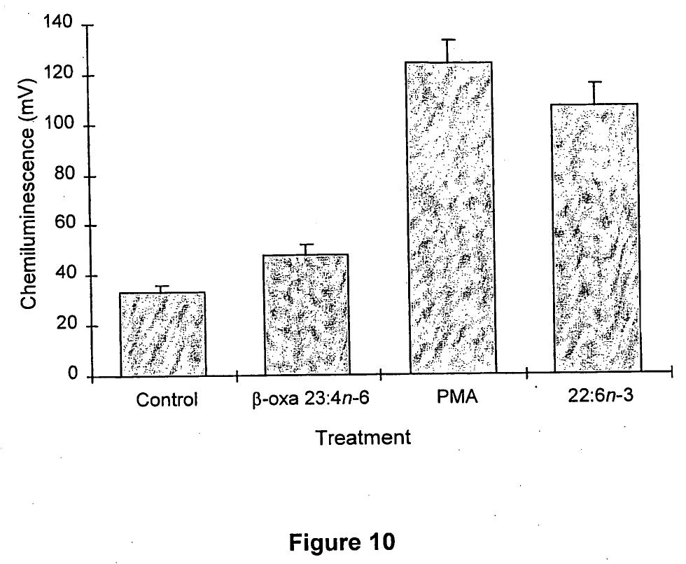

Unlike natural PUFA, the β-oxa and β-thia compounds are not readily β-oxidized and hence show high levels of intracellular stability (Pitt et al, 1998 supra). Compared with 20:4n-6 and 22:6n-3, β-substituted PUFA were found to be weak at stimulating the oxygen radical production in human neutrophils. In the case of MP3 (β-oxa 23:4n-6), a concentration of up to 30 μmol/l failed to cause any significant respiratory burst (chemiluminescence response), while the same concentration of 22:6n-3 produced marked responses, similar to the strong neutrophil activator, PMA (FIG. 10).

### Effects on TNF-Induced Up-Regulation of Neutrophil Adherence to HUVEC

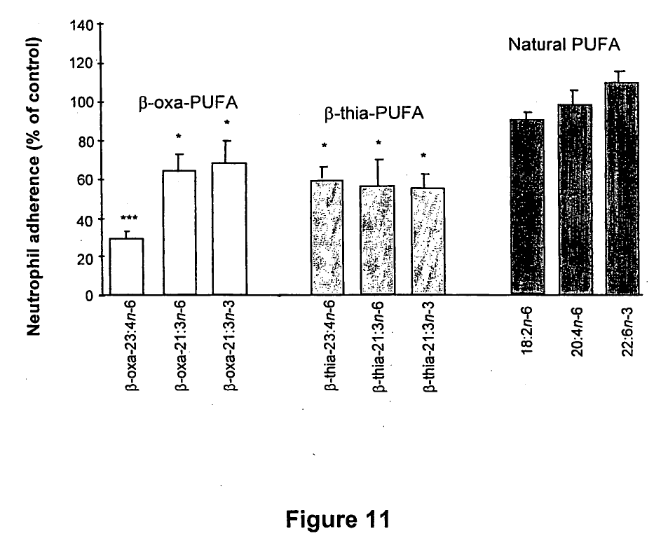

Data in FIG. 11 show that pre-treatment of HUVEC for 1 hr with β-oxa-PUFA (β-oxa-23:4n-6, β-oxa-21:3n-6, β-oxa-21:3n-3) or β-thia-PUFA (β-thia-23:4n-6, β-thia-21:3n-6 β-thia-21:3n-3) inhibited their ability to be stimulated by tumour necrosis factor-α (TNF-α) for enhanced neutrophil adhesion. In contrast, pre-treatment with the naturally-occurring PUFA, 20:4n-6, octadecadienoic acid (linoleic acid, 18:2n-6) and 22:6n-3, had no significant effect on the cytokine-induced adhesion of leukocytes to HUVEC. Although the fatty acids were presented to the cells with ethanol as diluent (0.1% v/v final concentration), similar results were obtained using mixed fatty acid-DPC micelles. Trypan blue exclusion and lack of [⁵¹Cr] chromate release from labeled cells showed that the cells remained viable under these experimental conditions. Furthermore, the engineered fatty acids did not affect DNA synthesis, glucose metabolism and G3PDH mRNA expression in HUVEC. MP3 caused the greatest suppression of TNF-α-induced neutrophil adhesion to HUVEC (FIG. 11) and was, therefore, employed in further studies. The magnitude of the suppressive effect of -MP3 was dependent on-pre-treatment time and concentration with significant effects observed with a pre-treatment time of 1 hr and a concentration of ≧5 μmol/l. In addition, β-oxa 23:4n-6 inhibited the increase in neutrophil adhesion to HUVEC induced by bacterial lipopolysaccharide (LPS) or PMA.

Effects of Derivatives of MP3 (β-oxa-23:4n-6)

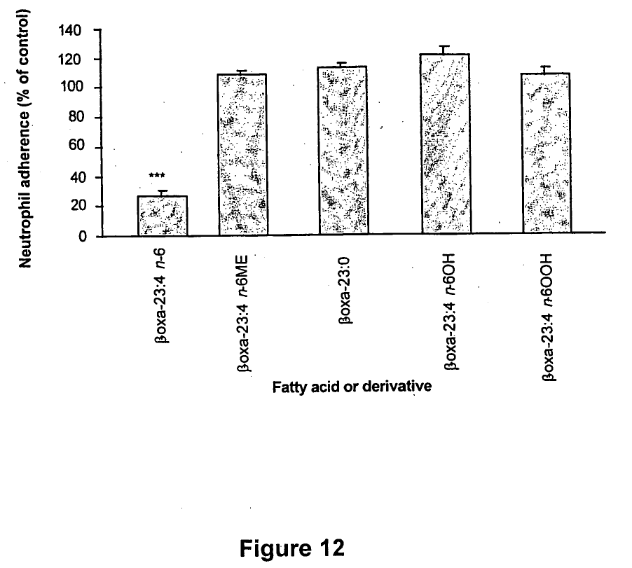

Derivatization of MP3 to methylated, saturated and 18-monohydroxy- and hydroperoxy-forms abolished its inhibitory effect on TNF-α-stimulated neutrophil adhesion to HUVEC (FIG. 12), demonstrating not only the specificity of the parent molecule but also that the structure of the parent molecule is critical for activity.

### Effects on TNF-Induced Expression of Adhesion Molecules on EC.

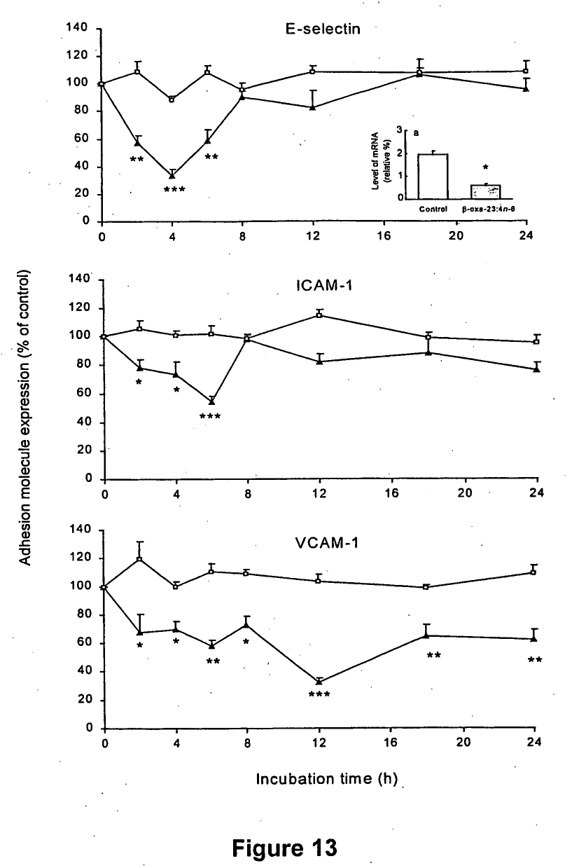

The inhibitory effect of β-oxa-23:4n-6 on adhesion was consistent with the ability of β-oxa-PUFA to suppress the TNF-α-induced expression of E-selectin (CD62E), intercellular adhesion molecule-1 (ICAM-1; CD54) and vascular cell adhesion molecule-1 (VCAM-1; CD106) adhesion molecules on HUVEC. As shown in FIG. 13, maximum inhibition of TNF-α-stimulated E-selectin, ICAM-1 and VCAM-1 expression was observed after 4, 6 and 12 hr of cytokine treatment respectively, after which there was recovery (particularly in the case of E-selectin and ICAM-1) up to 24 hr. The ability of the cells to regain their capacity to express adhesion molecules confirms that the synthetic fatty acid did not affect their viability. β-oxa-23:4n-6 inhibited the expression of E-selectin, ICAM-1 and VCAM-1 molecules in a concentration-dependent manner, which corresponded with the levels required to reduce neutrophil adherence. 20:4n-6 had no significant effect on HUVEC adhesion molecule expression compared with β-oxa-23:4n-6. TNF-α-induced increase in expression of E-selectin mRNA was found to be substantially depressed by β-oxa-23:4n-6 treatment (FIG. 13). β-oxa-23:4n-6 also inhibited LPS and PMA-induced, up-regulation of E-selectin, ICAM-1 and VCAM-1 induced by these agonists.

In Vivo Activity of β-oxa-23:4n-6 (MP3).

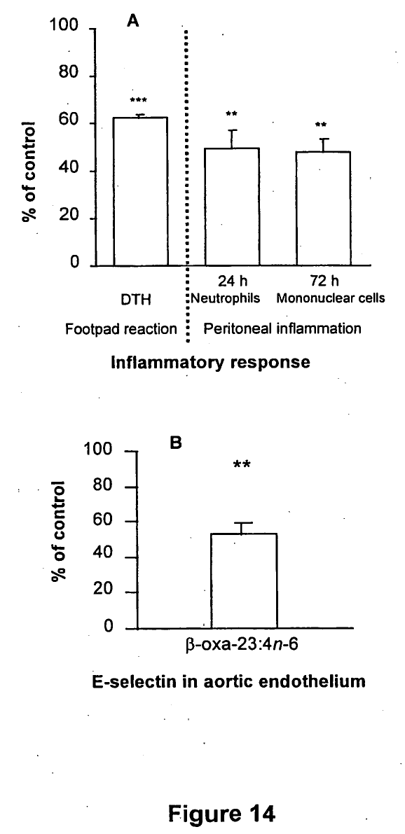

The β-oxa fatty acid was also found to be active in vivo. Mice sensitized with sheep erythrocytes were inhibited in ability to manifest a delayed type hypersensitivity reaction to this antigen if given an injection of β-oxa 23:4n-6 1 day prior to antigen challenge (FIG. 14A). This illustrated an effect on chronic inflammation probably through the inhibition of T cells and monocytes binding to the endothelium. When an acute inflammatory reaction (24 hr) was induced in mice by intraperitoneal injection of LPS, treatment with β-oxa 23:4n-6 inhibited the neutrophil influx (FIG. 11A). A similar inhibition of chronic inflammation was seen in terms of the inhibition of the mononuclear cell infiltrate after 72 hr (FIG. 14A).

The in vitro effects of β-oxa-23:4n-6 on adhesion molecule expression on endothelial cells was confirmed in mice treated with LPS (FIG. 14B). Mice treated with β-oxa-23:4n-6 showed a significant reduction in LPS-induced E-selectin expression in aortic endothelium.

### Metabolism of β-oxa 23:4n-6 in HUVEC.

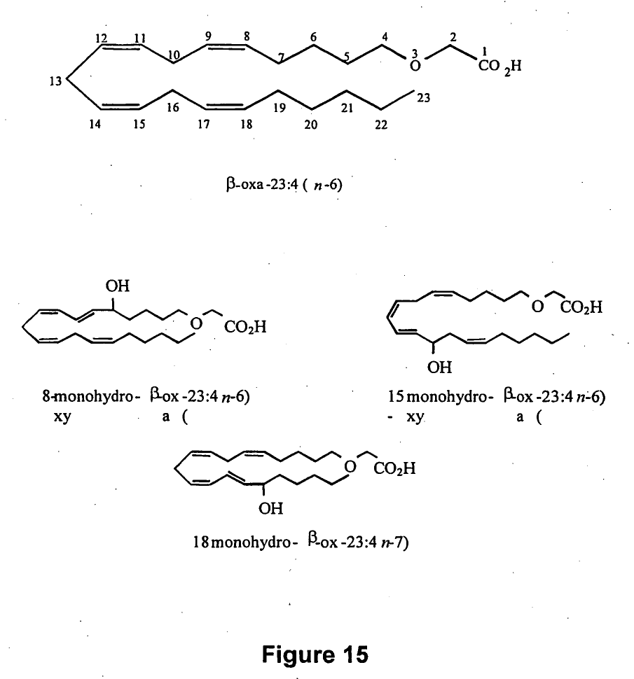

After incubation of HUVEC with β-oxa-23:4n-6 for 60 min, small amounts of three oxygenated fatty acids products were observed. The total ion chromatogram produced by electrospray MS of the combined products showed a molecular ion at m/z 365 (M⁺+1) (expected for mono-hydroxylated analogs of β-oxa-23:4n-6). Three daughter ions were found at m/z 264, 224 and 132 corresponding to loss of a C₆H₁₃O, C₉H₁₇O and C₁₆H₂₅O fragment, resulting from C₁₇-C₁₈, C₁₄-C₁₅ and C₇-C₈ bond cleavage, respectively. These fragments unambiguously confirm the identification of the three oxygenated products with mono hydroxyl group at carbons 18, 15, and 8 (FIG. 15). The 15-hydroxylated derivative was the major component (>90%). Pre-treatment of HUVEC with nordihydroguairetic acid (NDGA; a non-selective lipoxygenase inhibitor) markedly suppressed the formation of the oxygenated fatty acid products of β-oxa 23:4n-6, whereas indomethacin (a cyclooxygenase inhibitor) had no effect. Together, these results provide evidence that HUVEC converted β-oxa-23:4n-6 to 18-, 15- and 8-mono-hydroxylated derivatives (FIG. 15) by the lipoxygenase enzyme pathway, i.e. an enzymatic process rather than by auto-oxidation. Isomeric forms of monohydroxylated 20:4n-6 are synthesized from 20:4n-6 by cells via the action of stereo-specific lipoxygenase enzymes (Spector et al, Prog. Lipid. Res. 27:271-323, 1988). In HUVEC the lipoxygenase activity is mainly attributed to the 15-lipoxygenase (Buchanan et al, Haemostasis 18:360-375, 1988). The lipoxygenase positional isomer specificity is determined by the carbon chain length from the methyl end of the fatty acid substrates. Since β-oxa-23:4n-6 has three additional carbon atoms in its chain compared to 20:4n-6, it is likely that the 18-, 15- and 8-monohydroxylated derivatives of β-oxa-23:4n-6 are formed by the 15-, 12- and 5-lipoxygenases respectively in HUVEC.

### The Importance of the 12-LO in the Action of β-oxa 23:4n-6

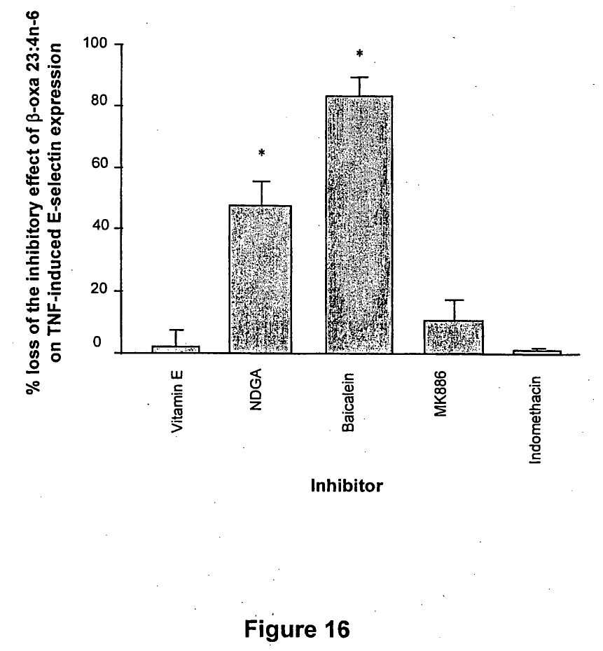

Confluent second-passage HUVEC in 96-well tissue culture plates were pre-treated with 10 μmol/l NDGA (non-selective lipoxygenase inhibitor); 10 μmol/l baicalein (a specific 12-lipoxygenase inhibitor); 500 nM MK886 (an inhibitor of the 5-lipoxygenase activating protein), 10 mmol/l indomethacin (a cyclooxygenase inhibitor); 10 μmol/l Vitamin E (an antioxidant); or diluent (control) for 15 min. The cells were then further incubated with 20 μmol/l β-oxa-23:4n-6 or diluent (control) for 60 min followed by TNF-α (125 U) for 4 h. The expression of E-selectin adhesion molecule was determined by ELISA. While none of the inhibitors/antioxidants affected the ability of TNF to enhance E-selectin expression on HUVEC, the ability of β-oxa 23:4n-6 to suppress the action of TNF was reduced when the cells were pre-treated with either NDGA or baicalein but not with indomethacin, Vitamin E or MK886 (FIG. 16). This indicated that conversion of β-oxa-23:4n-6 to an oxygenated product(s) via the 12-lipoxygenase pathway was important for the inhibitory activity of the fatty acid. It is unlikely that oxygenated products formed by the 15-lipoxygenase are involved in the inhibitory action of β-oxa-23:4n-6 because the 18-monohydroxy/hydroperoxy derivatives were inactive (FIG. 12).

### Effects of β-oxa 23:4n-6 (MP3) on TNF-Induced Activation of Intracellular Signalling Molecules.

Examination of the effects of β-oxa 23:4n-6 on intracellular signalling molecules involved in TNF-induced expression of these adhesion molecules, showed that pre-treatment of HUVECs with the fatty acid did not affect the ability of TNF to stimulate p38, ERK and JNK.

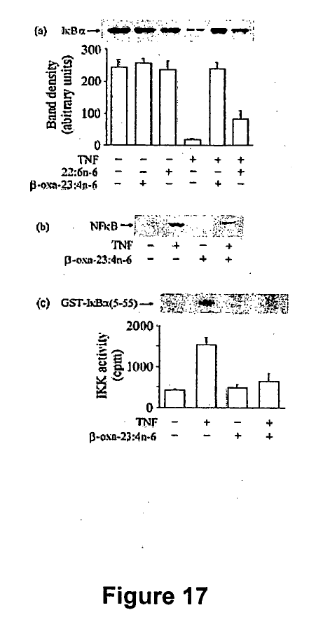

The effects of the β-oxa PUFA on the IKK-NFκB pathway were also examined which is important in the stimulation of expression of adhesion molecules on endothelial cells (Read et al, J. Biol. Chem. 272:2753-61, 1997). In this pathway, IκB, which ordinarily sequesters NFκB in the cytoplasm, is phosphorylated by IKK. This phosphorylation targets IκB for degradation, thereby allowing the nuclear translocation of NFκB. HUVEC pre-treated with β-oxa 23:4n-6 showed marked inhibition of IκBα degradation (>92%) induced by TNF (FIG. 17A). In comparison, the same concentration of DHA caused less than 50% inhibition of TNF-stimulated IκB degradation (FIG. 17A).

The effects of β-oxa 23:4n-6 on TNF induced activation of NFκB was confirmed by examining the translocation of the NFκB to the nucleus. The data showed inhibition of translocation of NFκB to the nucleus (FIG. 17B)

To see whether the effects of β-oxa 23:4n-6 on IκBα degradation could be due to inhibition of IKK activation, cells were pre-treated with β-oxa 23:4n-6, then stimulated with TNF and assayed for IKK activation. The results showed that β-oxa 23:4n-6 significantly inhibited the activation IKK (FIG. 17C).

The data demonstrate that by placing an oxygen or sulphur atom in the β-position of a PUFA, molecules can be generated which vary in biological activities from the natural n-3 PUFA. An important characteristic of the β-oxa/β-thia-compounds was their greatly reduced ability to stimulate the neutrophils respiratory burst but which retained or increased anti-inflammatory properties displayed by the n-3 PUFA. The β-oxa and β-thia PUFA significantly decreased the agonist-induced increase of neutrophil adhesion to the endothelium, while 20:4n-6 and 22:6n-3 showed no inhibition of this response under these conditions. However, longer term exposure of HUVEC to 22:6n-3 had previously been shown to decrease up-regulation of adhesion properties of these cells (De Caterina et al, Arterioscler Thromb. 14:1929-1936, 1994, Weber et al, Arterioscler Thromb. Vasc. Biol. 15:622-628, 1995). The most active of these newly synthesized compounds was β-oxa 23:4n-6. The corresponding β-thia 23:4n-6 was less active than this β-oxa compound. This illustrates how fatty acids bearing the same structural elements can vary dramatically in activity depending on whether these contain an oxygen or sulphur atom in the β-position. The novel PUFA and in particular β-oxa 23:4n-6, are therefore, similar in biological properties to the 15-HPETE which was shown to lack the ability to stimulate oxygen radicals in neutrophils but inhibited leukocyte adhesion to endothelial cells (Huang et al, 1997 supra, Sethi et al, J. Lab. Clin. Med. 128:27-38, 1996) and TNF production by macrophages (Ferrante et al, 1997 supra).

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Buchanan et al, Haemostasis 18:360-375, 1988
Chou et al, Curr Biol. 8:1069-77, 1998
Costabile et al, J. Immunol. 167:2980-2987, 2001
Couper et al, Diabetologia 37:533-535, 1994
De Caterina et al, Arterioscler Thromb. 14:1929-1936, 1994
Dekker et al, Biochem J. 347:285-289, 2000
Ferrante et al, Clin. & Exp. Immunol. 38:70-76, 1979
Ferrante et al, J. Immun. Methods 36:109-117, 1980
Ferrante and Thong, J. Immun. Methods 48:81-85, 1982
Ferrante et al, J. Clin. Invest. 99:1445-1452, 1997
Ferrante et al., In The Neutrophils: New outlook for the old cells [Ed Garbilovich] Imperial College Press 4:79-150, 1999
Hii et al, J. Biol. Chem. 266:20238-20243, 1991
Hii et al, J. Biol. Chem. 270:4201-4204, 1995
Hii et al, J. Biol. Chem. 273:19277-19282, 1998
Huang et al, Circ. Res. 80:149-158, 1997
Jaken et al, Bioessays 22:245-254, 2000
Jersmann et al, Infect. Immun. 69:1273-1279, 2001
Jirousek et al, J. Med. Chem. 39:2664-2671, 1996
Kikkawa et al, Diabetologia 37:838-841, 1994
Korchak et al, J. Biol. Chem. 269:30281-30287, 1994
Koya et al, J. Clin. Invest. 100:115-126, 1997
Koya et al, Diabetes 47:859-866, 1998
Lallena et al, Mol. Cell. Biol. 19:2180-2188, 1999
Lee et al, Proc. Natl. Acad. Sci. USA 95:9319-9324, 1998
Li et al, J. Clin. Invest. 97:1605-1609, 1996
Musial et al, J. Biol. Chem. 270:21632-21638, 1995
Pitt et al, Synthesis 11:1240-1242, 1997
Pitt et al, Chem. Phys. Lipids. 92:63-39, 1998
Powell et al, J. Immunol. 159:2952-2959, 1997
Read et al, J. Biol. Chem. 272:2753-61, 1997
Robinson et al, Biochem J. 336:611-617, 1998
Scivittaro et al, Am. J. Physiol. 278:F676-F683, 2000
Sethi et al, J. Lab. Clin. Med. 128:27-38, 1996
Sinclair and Gibson (Eds) Invited papers from the Third International Congress, American Oil Chemists' Society, Champaign, Ill., 1-482, 1992
Spector et al, Prog. Lipid Res. 27:271-323, 1988
Suzuma et al, J. Biol. Chem. 277:1047-1057, 2002
Way et al, Diabetic Medicine 18:945-959, 2001
Weber et al, Arterioscler Thromb. Vasc. Biol. 15:622-628, 1995
Xia et al., J. Clin. Invest. 98:2018-2026, 1996

1. A method for the treatment or prophylaxis of a condition selected from a NFκB related or associated condition, a PKCβ related or associated condition, vascular or immunological conditions such as diabetes, inflammation, neurological conditions, cardiovascular disease and pain in a subject said method comprising administering to said subject an effective amount of a compound having the structure of Formula (I): 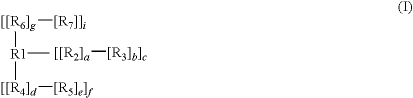
wherein
R₁ is a saturated or unsaturated hydrocarbon chain of from about 9 to about 26 carbon atoms and which is optionally carries one or more of a oxa, thia, hydroxy, hydroperoxy, epoxy and peroxy substitution;
each of R₂, R₄ and R₆ is selected from O₂, NO, NO₂, S(O)ₓ, C(H)_{y}, H, COOH, P(X)_{δ}(Y), N(H)_{z}, C═O, OH, 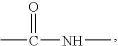
C₁₋₆ alkyl, C₁₋₆ alkoxy, amino, mono-acid di-C₁₋₆ alkylamino, C₁₋₆ alkylthio, S(O)ₓ—C₁₋₃ alkyl, C₁₋₆ alkoxycarbonyl, halo selected from fluoro, chloro, bromo and iodo, oxo, amidino and guanidino, C₂₋₁₂ alkenyl, C₂₋₁₂ alkynyl, aryl, heteroaryl and cyano, wherein x and z are 0, 1 or 2 and y is 0, 1, 2 or 3 and X is O, S or NR₈, Y is OR₉, SR₁₀ or NR₁₁R₁₂ and R₈, R₉, R₁₀, R₁₁ and R₁₂ are selected from H, alkyl, alkenyl, alkynyl, aryl and heteroaryl, δ is 0 or 1;
each of R₃, R₅ and R₇ is respectively [(CH₂)ⱼ(COOH)ₖ]ₗ, [(CH₂)ₘ(COOH)ₙ]ₒ and [(CH₂)ₚ(COOH)_{q}]ᵣ, wherein each of j, m and p is 0, 1, 2, 3, 4, 5 or 6, each of k, n and q is 0, 1 or 2,
and each of l, o and r is 0 or 1,
each of c i and f is 0 or 1 or 2;
each of a, d and g is 0 or 1 or 2;
each of b, e and h is 0 or 1 or 2;
said administration being for a time and under conditions sufficient to prevent the condition or to ameliorate one or more symptoms of the condition.

2. The method of claim 1 wherein the subject is a mammal.

3. The method of claim 2 wherein the mammal is a human.

4. The method of claim 1 wherein in Formula (I) each of i, c and f is 0 (zero), two of i, c and f is 0 (zero) or one of i, c and f is 0 (zero); or each of i, c and f is 1; two of i, c and f is 1 or one of i, c and f is 1; or each of i, c and f is two, two of i, c and f is two, or one of i, c and f is two.

5. The method of claim 1 wherein in Formula (I) each of g, a and d is 0 (zero), two of g, a and d is 0 (zero) or one of g, a and d is 0 (zero); or each of g, a and d is 1; two of g, a and d is 1 or one of g, a and d is 1; or each of g, a and d is two, two of g, a and d is two, or one of g, a and d is two.

6. The method of claim 1 wherein in Formula (I) each of h, b and e is 0 (zero), two of h, b and e is 0 (zero) or one of h, b and e is 0 (zero); or each of h, b and e is 1; two of h, band e is 1 or one of h, band e is 1; or each of h, band e is two, two of h, b and e is two, or one of h, b and e is two.

7. The method of claim 1 wherein the L-amino acid is selected from alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and valine.

8. The method of claim 1 wherein a chemical analog of an amino acid is selected from α-aminobutyric acid, α-amino-α-methylbutyrate, aminocyclopropane-, carboxylate, aminoisobutyric acid, aminonorbornyl-, carboxylate, cyclohexylalanine, cyclopentylalanine, D-alanine, D-arginine, D-aspartic acid, methylmethionine, D-cysteine, N-methylnorleucine, D-glutamine, D-glutamic acid, methylornithine, D-histidine, N-methylphenylalanine, D-isoleucine, D-leucine, D-lysine, D-methionine, D-ornithine, D-phenylalanine, D-proline, D-serine, D-threonine, D-tryptophan, D-tyrosine, D-valine, D-α-methylalanine, D-α-methylarginine, D-α-methylasparagine, D-α-methylaspartate, D-α-methylcysteine, D-α-methylglutamine, D-α-methylhistidine, D-α-methylisoleucine, D-α-methylleucine, D-α-methyllysine, D-α-methylmethionine, D-α-methylornithine, D-α-methylphenylalanine, D-α-methylproline, D-α-methylserine, D-α-methylthreonine, D-α-methyltryptophan, D-α-methyltyrosine, D-α-methylvaline, D-N-methylalanine, D-N-methylarginine, D-N-methylasparagine, D-N-methylaspartate, D-N-methylcysteine, D-N-methylglutamine, D-N-methylglutamate, D-N-methylhistidine, D-N-methylisoleucine, D-N-methylleucine, D-N-methyllysine, N-methylcyclohexylalanine, D-N-methylornithine, N-methylglycine, N-methylaminoisobutyrate, N-(1-methylpropyl)glycine, N-(2-methylpropyl)glycine, D-N-methyltryptophan, D-N-methyltyrosine, D-N-methylvaline, γ-aminobutyric acid, L-t-butylglycine, L-ethylglycine, L-homophenylalanine, L-α-methylarginine, L-α-methylaspartate, L-α-methylcysteine, L-α-methylglutamine, L-α-methylhistidine, L-α-methylisoleucine, L-α-methylleucine, L-α-methylmethionine, L-α-methylnorvaline, L-α-methylphenylalanine, L-α-methylserine, L-α-methyltryptophan, L-α-methylvaline, N—(N-(2,2-diphenylethyl)carbamylmethyl)glycine, and 1-carboxy-1-(2,2-diphenyl-ethylamino)cyclopropane.

9. The method of claim 1 wherein the cytokine is selected from BDNF, CNTF, EGF, EPO, FGF1, FGF2, FGF3, FGF4, FGF5, FGF6, FGF7, FGF8, FGF9, FGF10, FGF11, FGF12, FGF12, FGF13, FGF14, FGF15, FGF16, FGF17, FGF18, FGF19, FGF20, FGF21, FGF22, FGF23, G-CSF, GM-CSF, IFNα, IFNβ, IFNγ, IL1, IL2, IL3, IL4, IL5, IL6, IL7, IL8, IL9, IL10, IL11, IL12, IL13, IL14, IL15, LIF, MCP1, MCP2, MCP3, MCP4, MCP5, M-CSF, MIP1, MIP2, NGF, NT 3, NT4, NT5, NT6, NT7, OSM, PBP, PBSF, PDGF, PF4, RANTES, SCF, TGFα, TGFβ, TNFα, TNFβ, TPO, VEGF, GH, insulin and the like.

10. The method of claim 1 wherein the apoptotic proteins is selected from A1, A9, A20, A46R, A52R, A53, A238L, Aac11, AATF, AATYK, ABIN1, ABIN-1, ABIN2, Acidic Sphigomyelinase, Acinus, Act1, ACT2, Activin, AD3LP, AD5, ADAR, adrenomedullin, aggrecan, AMAM17, 33, AI1, AIF, AILIM, AIM2, AIR, AITR, Akt, ALCAM, ALG2, ALG3, ALG4, ALP, Alix, Armadillo, AMAC1, AMH, AMID, Amida, angiotensinogen, Ankyrin, ANT1, AO7, AP1, Apaf-1, APC, APC2, APCL, APE1820, APJ, APO-1, APO-2, APO-3, Apopain, APP1, APP2, Apr, APRIL, ARA54, ARC, ARF, arkadia, ARIH1, 2, ASC, Ash2, Ask1, Ask2, ASPP1, ASPP2, AT2R1, AT2R2, ATAR, ATF1, ATF2, ATF3, ATF4, ATM, atona, ATR1, AUF1, Aven, AVP, AvrA, AvrBsT, Axam, Axin, Axin 2, Axi, b-catenin, b-TrCP, B28R, B7-1, B7-2, B7h2, B7RP1, Bach2, Bad, BAFF, BAG-1, -2, -3, -4, -5, Bak, BALF1, Bam32, BAP-1, BAP31, BAP29, BAR, BARD1, BAT3, Bax, BBc3, BCA1, BCAN, Bcl-2, BCL2, Bcl-3, Bcl-10, BCL10, Bcl-G, Bcl-Rambo, Bcl-w, Bcl-x, beclin, BEHAB, BERP, Bfl-1, BFL1, BG1, BG2, BG4, BG5, BHP1, BHRF1, BI-1, Bid, Bif-1, Bik, Bis, Bim, Bimp-1, Bimp1, Bimp2, Bimp3, BIR1, BIRP, BL-CAM, BLC, Blk, BLNK, BLR1, BLyS, BMI-1, BmP109, BNIP3, BNIP3a, BNIP3L, Bok, bone sialoprotein, bonus, Boo, BPI, BRAL1, BRAG-1, BRAP, Bravo, BRCA1, BRN3a, BRN3b, BRN3c, brevican, BPR, BSAC, BUFFY, C1q, C1r, C1s, C2, C3, C4a, C4b, C5, C6, C7, C8a, C8b, C8g, C9, C1qBP, C3aR, C4BPa,b, C5R1, CR2, CIITA, C5L, c-E10, c-FLIP, c-Fms, c-Fos, c-IAP1, cIAP1, c-IAP-1, c-IAP2, cIAP2, c-IAP-2, c-Jun, c-Myc, c-Rel, Cactus, CAD, cadherin, E, N, P, VE, calcineurin, CARD4, CARD7, CARD9, CARD10, CARD11, CARD12, CARD14, CARDIAK, Carmal, CARMA-1, CARMA2, CARMA3, CARMA, CARMEN, CAP1, CAR1, CART1, CAS, CAS-L, Caspase-1, -2, -3, 4, -5, -6, -7, -8, -9, -11, -12, -13, -14, Casper-1, -2, -3, -4, -5, -6, -7, -8, -9, -10, -11, -12, -13, -14, -15, -16, -17, -18, -19, -20, -21, -22, -23, -24, -25, -26, -27, -28, CASH, CBL, CBL-B, CBL-C, CC-CKR-6, CCF, CCL, CCPI, CCRs, CD2, CD3, CD4, CD5, CD6, CD7, CD8, CD9, CD11, CD14, CD18, CD19, CD20, CD21 (CR2), CD22, CD23, CD25, CD27, CD27L, CD28, CD28LG1, CD28LG2, CD29, CD30, CD31, CD32, CD33, CD34, CD35, CD36, CD40, CD40L, CD41, CD43, CD44, CD45, CD46, CD47, CD48, CD49, CD50, CD53, CD54, CD55, CD56, CD58, CD59, CD61, CD62E, L, H, CD66, CD63, CD64, CD66a-e, CD67, CD70, CD72, CD74, CD79a, b, CD80, CD84, CD85a-m, CD86, CD88, CD89, CD90, CD92, CD94, CD95, CD96, CD97, CD99, CD100, CD101, CD102, CD104, CD105, CD106, CD108, CD112, CD115, CD116, CD117, CD119, CD120a, b, CD121a, b, CD122, CD123, CD124, CD125, CD126, CD127, CD128a, b, CD130, CD131, CD132, CD134, CD135, CD136, CD137, CD140a, CD140b, CD143, CD144, CD146, CD147, CD148, CD150, CD151, CD152, CD153, CD154, CD155, CD158a-z, CD159, CD160, CD161, CD162, CD166, CD178, CD180, CD183, CD184, CD195, CD197, CD207, CD229, CD244, CDC2, CDC25, CDC42, CDK1, CDK2, CDK5, CDM, CEA, CEAL, CEACAM1, 6, C/EBP, CED1, CED2, CED3, CED4, CED5, CED6, CED7, CED8, CED9, Ced-9, CED10, CED11, CED12, CED, CEP-1, CES1, CES2, CES3, CETP, CeTRAF, Cezanne, CGR19, CGRP, Che1, Che-1, CHFR, chemokines, CHOP, CHUK, cIAP1, cLAP2, c-IAP1, c-IAP2, c-IAP-1, c-IAP-2, CIDE-A, -B, CIKS, CIN85, CIP-1, CIPER, CISK, Ckb-8, CKR1, 2, 3, 4, 5, CKRL1, Clan, CLAP, CLARP, CMD1, CMH1, CMKBR1, 2, 3, 4, 5, 6, CMPD1, conductin, Cop9 subunit 3, COP11, COPS3, COPS5, COT, COX-1, COX-2, CPAN, CPP32, CPZ, CRADD, CRAF1, CR8, CREB, CREM, Crk-II, crinkled, crmA, crmB, CSBP1, CSMF, CSN3, Csp-1, -2, -3, CSPG2, 3, Csx, CTACK, CTAP3, CTGF, CTLA4, cytochrome c, cytosolic PL A2, CXCLs, CXC-R3, DAAM1, Dad1, DAD-1, Damm, DAP1, DAP3, DAP5, DAP12, DAP kinase 1, DAPP1, DAYDREAM, DAXX, Dborg1, dCAD, DCCK1, DCP1, Dcp-1, DCP2, Dcp-2, DcR 1,2,3, DD2, Decay, DED, DEDAF, DEDD, DEDD2, dedprol, defensin, DEFT, dFADD, DFF, DFF35, DFF40, DFF45, DG17, Diablo, DIAP1, DIAP2, Dickkopf, DIF, DIF2, DIHA, DIK, Drosophila IKK, PKCdelta-interacting protein kinase, DIO1, DIP, disheveled, diubiquitin, DKK1,2,3,4, DLAK, DLK, DMDL, DNase II, Diva, DONG1, Dorsal, DP1, DP2, DP5, Drob1, DRP-1, DocA, dock188, Dok1, Doom, dorfin, DR3,4,5,6, DRAK 1, 2, DREAM, DREP-1, -2, -3, -4, DrICE, DRONC, DRP1, DTR, DTS, DUSP, E1.1, E1B 19K, E10, E2Fs, E4BP4, E4ORF4, E8, E4, E48, E3RS, eae7, Ear7, EBAF, EBI1, EBP1, EBI6, ECSIT, EDA, EDAR, Edradd, EFP, EGL1, Egr1, 2, 3, EHF, eIF-2aK, Eiger, ELAM, ELF2, ELK1-4, EMR1, ENA78, Endofin, Endoglin, Endophilin B1, endothelin, ENG, eNOS, eotaxin 1,2, ERN1, ERICE, ES18, Ets-1, -2, ER81, ErbAa, ERG, ERM, ESE2, Eskine, ETV1, 2,3,4,5,6, exodus-1, 2, 3, FADD, Fas associated via death domain, FAF1, FAIM, FAN, FANCC, Fas, FAST, FAT10, fb1, FCAR, FELL, FEM-1, FEM-2, FHR1, 2, 3, 4, 5, FKBPs, FIGF, FIL1d, e, eta, zeta, FIP2, FIP-2, FIP3, FIP-3, FKSG2, FIST, FKHL12, FKHR, FKHRL1, FLAME-1, FLAME-3, FLAME3, FLASH, FLDED-1, FLI-1, FLI1, FLICE, FLICE2, FLICE-2, FLIP, FLT3L, Fliz1, Fln29, Fms, Fnk, fortilin, Fos, FOXO1A, FOXO3A, FOXE3, FPV039, Fra1, Fra2, Fractalkine, FRAP, FREAC8, Frizzled, Fzd, Fz, FRING, FRP1, 2, 3, FRP1(ATR), frpHE, FRZB-PEN, Fsp27, FUS, FUS6, Fusin, FXY, FY, G-coupled receptors, G10P1, G25K, G4R, G6C, G6E, GADD34, GADD45, GADD153, GATA1,2,3,4,5,6, GBP2, GCP2, GDFs, gelsolin, Gfi-1, Gfi1, GFRP1, GILZ, gingipain, GITR, GL50, glycodelin A, GM2A, gp34, GPR5, GPR9, GPR-9-6, Granzyme B, Grim, GRMP, Groa, Grob, GRS, GSKbeta, H2TF1, H731-like, Hakai, HB-EGF, Hck, HF1, HFB30, HFL3, HHARI, hIAP-1, hIAP1, Hid, HIF1 alpha, HIP1, HIP116, HIPPI, HIPK1,2,3, histamine receptors, HIVEP1, 3, HIV-EP1, HLTF, HM85, HM89, HM145, HMR, HNRPD, HRD1, Hrk, HtrA2, Huntingtin, HVEM, HVEML, HYP, IAP-1, IAP1, IAP2, IAP, iAPP, ICAD, ICBP90, ICE, ICEBERG, ICE-LAP3, ICE-LAP6, ICErel-II, ICErel-III, Ich1, ICH-1, Ich2, ICH-2, Ich3, ICH-3, ICOS, I-TRAF, I-FLICE, IEX-1m IFI, IFIT1, 2, 3, 4, IFP35, IgE Fc Receptor, IGF1 and its receptor, IGFBP-3, IKAP, Ikaros, IKB-1, IkB a, b, e, IKKAP1, IKK 1,2, IKK a,b, IKKg, interleukins, interleukin receptors, IL1 antagonist, anti-IL1, IL1RacP, IL8R1, ILA, ILC, ILP, ILP-1, ILP-2, ILT1-11, ING1, ING2, ING3, Inhibin, INK4, INK4A, integrin, IP10, INP10, IP30, Ipaf, IRAK, IRAK2, IRAM-M, IRE1, RE1p, IRE, IRF, IRTA1-5, ISGF3g, ITA, It, Jab1, Jak1, 2, 3, JDP2, JIK, JN, K, K13, KARAP, KBF 1, 2, 3, KDS, KE05, KET, kf-1, KIAP, Killer, KIR2DL1-5, KIR2DS1-6, KROX2, L-Myc, lactalbumin alpha, LAG1, LAIR1, LALBA, LAM, LAP1, LAP3, LAR, LARD, LARC, LATS1, 2, LBP, Lck, LCP2, LD78b, LEFTY, LESTR, Leu1, Leu8, Leu14, leukotactin, LFA3, LFG, LICE, LICE2, LIF, LIGHT, LIR1, 2, 3, 4, 5, 6, 7, 8, Livin, LMP1, LMW5-HL, LOK, Lot1, LRDD, LRP, Low affinity NGFR, LTa, LTb, LTbR, LTP2, Ly63, lymphotactin, Ly1, Lyf1, Lysozyme, Lyt-10, LYVE1, LZK, M11, M159L, M160L, MA-3, MACH, Mad, Mad3, MADD, Maf, c-Maf, makorin, MAL, MALT, MAP-1, MAPKKKKs, MAPKKKs, MAPKKs, MAPKs, Math1, Max, MBD4, MBLR, MBP1, MCL1, Mch2, Mch3, Mch4, Mch5, Mch6, MCP1, 2, 3, MCP-1, Mda-7, MD-1, MD-2, Mdm2, Mdm4, MdmX, MDP62, mE10, MEF2a, MEKKs, Mel-18, MEMD, Meprin, metacaspase, MIC1, MID1, MIF, MIG, MIHC, MIP1, 2, 2a, 2b, MIP-T3, MIR, MIS, MITF, MKK6, MKL1, MKP1, ML-1, ML-IAP, MLN64, MLX, MMP-1,2,3,4,5,6,7,8,9,10,11,12,13,14,15,16, MNDA, MNT, Mob1, mod (mdg4), MORT1, MPIF1, 2, MRFP, MRIT, Msx1, Msx2, MTAP44, Mtd, mTOR, MUC1, MUC2, MUL, MURF-1,2,3, myp-nop30, MxA, MxB, Mxi1, Mxi2, MYAK, Myc, MyD88, MyD118, MYLK, myoblast city, N-Myc, NAF1, NAIP, NALP1, NALP2, NAP2, NBAK3, Nbk, NBS1, NCA, NCAM, NCC1, 2, 3, 4, NDG1, Neural Sphigomyelinase, Neuralin, NEMO, neogenin, Neurotactin, neurocan, NF-kB, NF-X1, NFATs, NFIL3, NFIL6, NFkB1, 2, NIP1, NIP2, NIP3, NIPK, NIK, Nix, NKAT1-9, NKX2-5, nNOS, Notch, NOD-1, NOD-2, nop30, Nor-1, NOS2, NOS2B, NOS3, Nov, Noxa, NP10, Np95, Npc2, NPY3R, Nr-CAM, NR3, NR13, Nr-13, NRAGE, NRIF1, nucleolin, Nur77, NY-REN-64, OCIF, ODF, ODFR, OIAS, ORF16, posteoprotegerin, OSX, OX40, OX40L, OPG, OPGL, Osi, osteonectin, osteoponti, p14, p16, p331NG1, p35, p38, p49, p49, p55, p52, p53, p53AIP1, p53DINP1, p55, p60, p62, p62Dok, p63, p65, p73, p75NTR, p84, p100, p105, p193, p202, PAC1, PACAP, PACT, PAF400, PAG-3, PAG608, PAK1, PAK2, PAK3, PAP1, PAR4, paracaspase, PARC, Park2, parkin, PARP, PAX2, 3, 5, 8, PBEF, PBP, PD1, PDGF, PEA15, Pellino, PERK, PERP, PEK, Pelle, PEX10, PF4, PGRP, PI3K, Pidd, PIK-1, PLAB, Plk, Plk3, PKC, PKR, PKY, PLAGL1, PLAIDD, PLA2, PLC, PLD, Pli, Pml, PMP41, POSH, PP1A, PP14, PP2Ca, PRKR, PRSS25, polycystin 1, porimin, PRG1, Prk, PRL, prolactin receptor, PS-1, -2, PSCA, PSMD11, 12, 13, PSP-C, PSK, PSSALRE, PTEN, PTK1, PTPs, PTP1C, PTP2C, PTP1G, PTPL1, PU.1, puckered, Pum, Q2/2, Rac, RAI, RANTES, RAX, Rb, Relish, RELT, Raf, RANK, RANKL, RAIDD, RBBP6, RBQ1, Rcm, Reaper, RelA, relaxin H1,H2,H3, RelB, Requiem, RFP, RFPL-1,2,3, RGS, RhoA, RICK, RIG-G, Ro52, Ro 60 kDa, ROC-1, 2, RORgamma, ROX, RIFF, RIP, RIP2, RIP3, RNM561, RNF, RP-8, RP8, RP105, Rpr, RRP5, RYBP, S9, S152, SAG, Salvador, SAP1, SAPK2A, Sara, SARP 1,2,3, Sav, Sca2, SCA-2, SCC-S2, SCF, SCDGF, SCM1, 1a, Scythe, SDF1, selectin L, E, P, SENP1, SENP2, sentrin/SUMO-specific protease, SETA, SFRP1, 2, 3, 4, 5, SFTP2, SFTPC, SGK, SGL, SGN5, SH2D1A, SHP1, 2, Siah, SIMPL, SIP27, SIP18, SIR2, SIVA, SLC, SLK, SLP-65, SLP-76, SLUG, Smac, SMADs, SMARCA3, SMN, SMT 3A, B, 3C, SNAIL, SNF2L3, SODD, somatostatin, Son3, SOX9, SP5, SP-C, SPARC, Sphigomyelinase, Smase, SPOP, SPP1, SPRK, Spatzle, SFRP1,2,5, SS-56, SSA, SSA1, SSA2, ST2L, stabilin 1,2, STATs, STCP1, STG6, STEP, STM-2, Stra3, STRICA, Substance P, SUMO1, survivin, SYK, SY, T cell receptor, T2BP, T6BP, TAB1, Tab2, Tabby, TACI, TACTILE, Tag7, tachykinin, TAJ, TAK1, Tak1, TALL-1, TANK, TAO1, TAO2, TARC, TBX1,2,3,4,10,18,19,20,21,22, TCA3, TCA-3, TC1, TC2, TCR, TCTP, TDAG51, TEAP, TECK, TEGT, TEL, (TEL1), TEL2 (TELb), telokin, TERF, TFT, TGb, TGFbeta 1,2,3, THG1, THRa, Thy-1, TIA1, TIAP, TIEG, TIF1, TIFgamma, TIL6, TIMP1,2,3, TIP49, Tip60, TIRAP, TIS, TLRs, TLS, TMS1, TNFa, TNFAIP3, A20, TNFAIP6, TNFb, TNF-C, TNFR1, TNFR2, TNFR-II, TNFRSF1-19, Toll, Tollo, Tollip, TONEBP, Toso, Tp44, TPL-2, TR3, TR2L, TRABID, TRADD, TRADE, TRAF1, TRAF1(Dm), TRAF2, TRAF2(Dm), TRAF3, TRAF4, TRAF5, TRAF6, TRAF6(Dm), TRAFamn, TRAIL, TRAIL-R2, TRAMP, TRANCE, TRC8, TRIAD1, 3, TRIF, TRIM, TRIP15, TRF-1, TRF-2, TRF1, TRF2, traube, TRDL-1, TRG, TRH, TRICK2, TRIP, Tristetraproline, TROY, TRRAP, TSC-22, TSC-22R, TTRAP, Tube, TUCAN, TWEAK, TX, TXBP151, TY, Tyk, UBCH7BP, UL36, UL37, Ulp, Unc5, UNC5h3, Urinary, stone protein (SPP1), USP7, usurpin, uterophi, vasopressin, Vav, vav1, vav2, vav3, vav-1, vav-2, vav-3, versican, vICA, VIAF1, vBcl-2, VEGI, VEGF, Ventroptin, VG-1, VG71, VHR, v-IAPs, VI, Warts, Wengen, WIG1, WISP-1, 2, 3, Wnt, WSL-1, WT1, WW45, WWOX, XAF1, XAP4, XCL1, 2, XEDAR, XIAP1, xRI, xRII, XICE, XICEa, XICE, Yama, YopJ, YY1AF, Zac, Zac1, ZAP70, ZBP89, zf3, ZFP26, ZFP127, ZH-DR, ZNF40, 124, 148, as TFs, ZNF144, 147, 179, 313, 364 as RING, ZIP-kinase, ZPR, 18 wheeler, 24.6K Glu/Pro-rich, 4-1BB, 4-1BBL, 4-1BB ligand, 53BP2, 7TM.

11. The methods of claim 1 wherein the pro-survival protein is selected from Bcl-2, Bcl-XL, Mcl-1 and A1.

12. The method of claim 1 wherein the compound is selected from

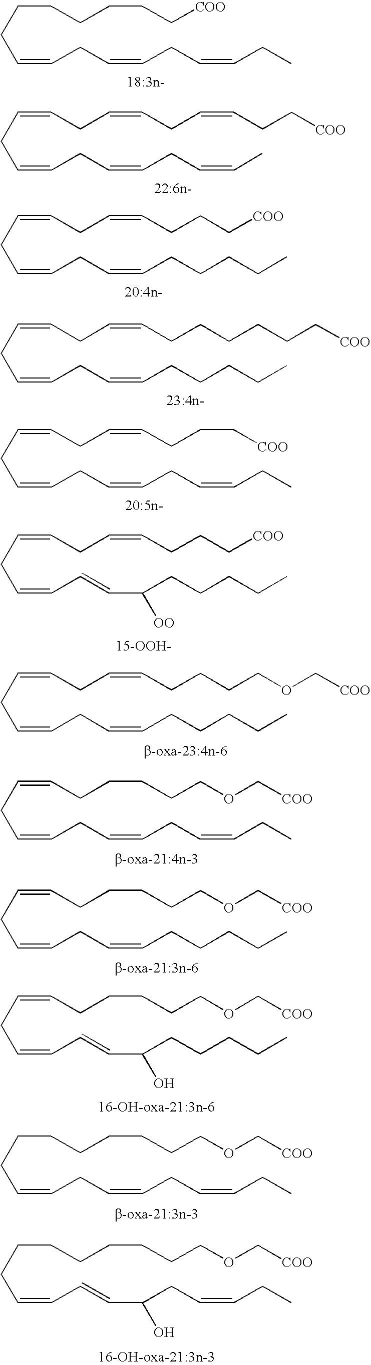

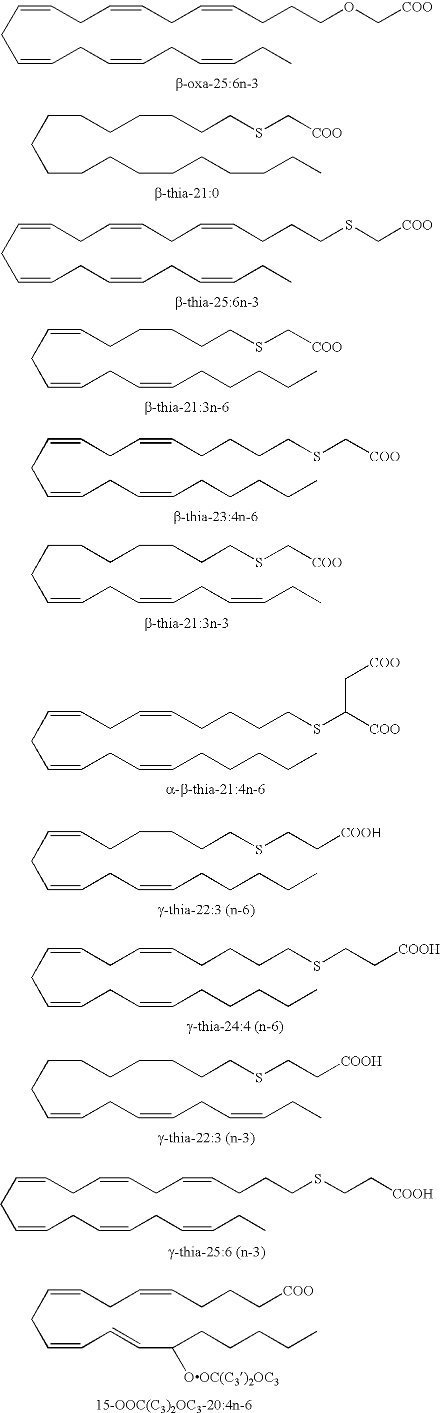

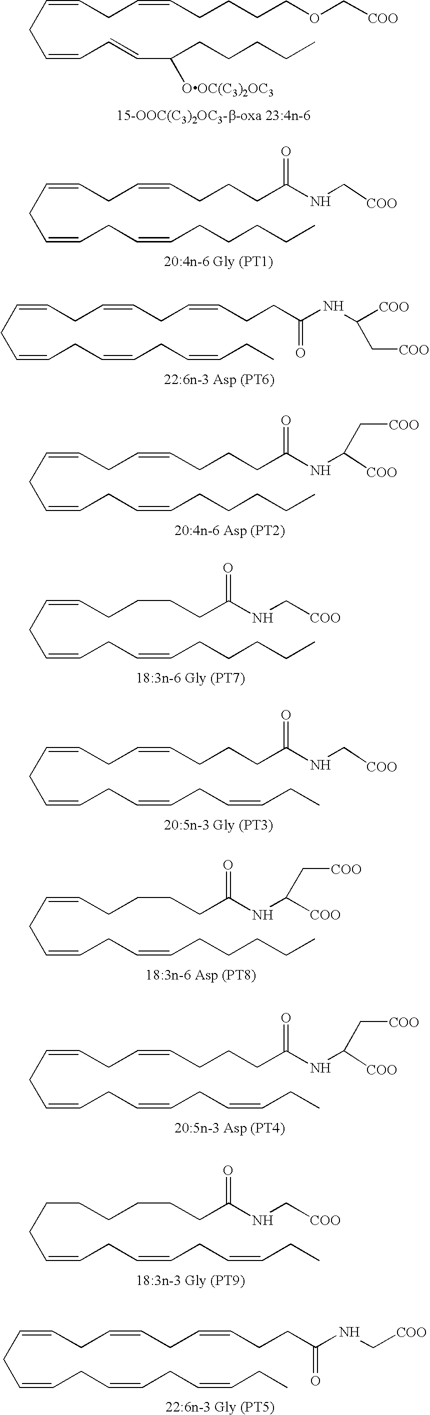

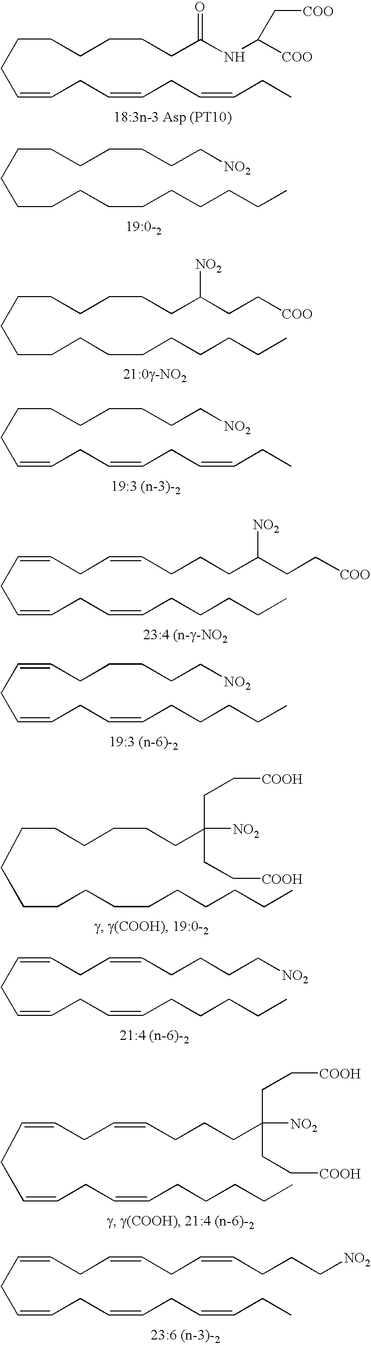

13. The method of claim 1 wherein the treatment is for pain including inter alia neuropathic or neurological pain, chronic pain, acute pain, migraine, headache inflammatory pain, postoperative pain, pain due to multiple sclerosis, Parkinson's disease or other neurological or autoimmune disorder or following or during periods of anxiety, delayed onset muscle soreness, burns or during or following infection or a convulsion, post-poliomyelitic pain, bipolar disorder, panic attack or epilepsy.

14. The method of claim 1 wherein the treatment is for depression, including major depression (single episode, recurrent, melancholic), atypical, dysthymia, subsyndromal, agitated, retarded, co-morbid with cancer, diabetes, or post-myocardial infarction, involutional, bipolar disorder, psychotic depression, endogenous and reactive, obsessive-compulsive disorder, or bulimia. In addition, NAALADase inhibitors can be used to treat patients suffering from pain (given alone or in combination with morphine, codeine, or dextroproposyphene), obsessive-compulsive personality disorder, post-traumatic stress disorder, hypertension, atherosclerosis, anxiety, anorexia nervosa, panic, social phobia, stuttering, sleep disorders, chronic fatigue, cognition deficit associated with Alzheimer's disease, alcohol abuse, appetite disorders, weight loss, agoraphobia, improving memory, amnesia, smoking cessation, nicotine withdrawal syndrome symptoms, disturbances of mood and/or appetite associated with pre-menstrual syndrome, depressed mood and/or carbohydrate craving associated with pre-menstrual syndrome, disturbances of mood, disturbances of appetite or disturbances which contribute to recidivism associated with nicotine withdrawal, circadian rhythm disorder, borderline personality disorder, hypochondriasis, pre-menstrual syndrome (PMS), late luteal phase dysphoric disorder, pre-menstrual dysphoric disorder, trichotillomania, symptoms following discontinuation of other antidepressants, aggressive/intermittent explosive disorder, compulsive gambling, compulsive spending, compulsive sex, psychoactive substance use disorder, sexual disorder, schizophrenia, premature ejaculation, or psychiatric symptoms selected from stress, worry, anger, rejection sensitivity, and lack of mental or physical energy.

15. The method of claim 1 wherein the treatment is for Moderate Mental Retardation, Severe Mental Retardation, Profound Mental Retardation, Unspecified Mental Retardation, Autistic Disorder, Pervasive Development Disorder NOS, Attention-Deficit Hyperactivity Disorder, Conduct Disorder, Group Type, Conduct Disorder, Solitary Aggressive Type, Conduct Disorder, Undifferentiated Type, Tourette's Disorder, Chronic Motor or Vocal Tic Disorder, Transient Tic Disorder, Tic Disorder NOS, Primary Degenerative Dementia of the Alzheimer Type, Senile Onset, Uncomplicated, Primary Degenerative Dementia of The Alzheimer Type, Senile Onset, with Delirium, Primary Degenerative Dementia of the Alzheimer Type, Senile Onset, with Delusions, Primary Degenerative Dementia of the Alzheimer Type, Senile Onset, with Depression, Primary Degenerative Dementia of the Alzheimer Type, Presenile Onset, Uncomplicated, Primary Degenerative Dementia of the Alzheimer Type, Presenile Onset, with Delirium, Primary Degenerative Dementia of the Alzheimer Type, Presenile Onset, with Delusions, Primary Degenerative Dementia of the Alzheimer Type, Presenile Onset, with Depression, Multi-infarct dementia, Uncomplicated, Multi-infarct dementia, with Delirium, Multi-infarct Dementia, with Delusions, Multi-infarct Dementia, with Depression, Senile Dementia NOS, Presenile Dementia NOS, Alcohol Withdrawal Delirium, Alcohol Hallucinosis, Alcohol Dementia Associated with Alcoholism, Amphetamine or Similarly Acting Sympathomimetic Intoxication, Amphetamine or Similarly Acting Sympathomimetic Delusional Disorder, Cannabis Delusional Disorder, Cocaine Intoxication, Cocaine Delirium, Cocaine Delusional Disorder, Hallucinogen Hallucinosis (305.30), Hallucinogen Delusional Disorder, Hallucinogen Mood Disorder, Hallucinogen Posthallucinogen Perception Disorder, Phencyclidine (PCP or Similarly Acting Arylcyclohexylamine Intoxication, Phencyclidine (PCP) or Similarly Acting Arylcyclohexylamine Delirium, Phencyclidine (PCP) or Similarly Acting Arylcyclohexylamine Delusional Disorder, Phencyclidine (PCP) or Similarly Acting Arylcyclohexylamine Hood Disorder, Phencyclidine (PCP) or Similarly Acting Arylcyclohexylamine Organic Mental Disorder NOS, Other or unspecified Psychoactive Substance Intoxication, Other or Unspecified Psychoactive Substance Delirium, Other or Unspecified Psychoactive Substance Dementia, Other or Unspecified Psychoactive Substance Delusional Disorder, Other or Unspecified Psychoactive Substance Hallucinosis, Other or Unspecified Psychoactive Substance Mood Disorder, Other or Unspecified Psychoactive Substance Anxiety Disorder, Other or Unspecified Psychoactive Substance Personality Disorder, Other or Unspecified Psychoactive Substance Organic Mental Disorder NOS, Delirium, Dementia, Organic Delusional Disorder, Organic Hallucinosis, Organic Mood Disorder, Organic Anxiety Disorder, Organic Personality Disorder, Organic Mental Disorder, Obsessive Compulsive Disorder, Post-traumatic Stress Disorder, Generalized Anxiety Disorder, Anxiety Disorder NOS, Body Dysmorphic Disorder, Hypochondriasis (or Hypochondriacal Neurosis), Somatization Disorder, Undifferentiated Somatoform Disorder, Somatoform Disorder NOS, Intermittent Explosive Disorder, Kleptomania, Pathological Gambling, Pyromania, Trichotillomania and Impulse Control Disorder NOS.

16. The method of claim 1 wherein the treatment is for Schizophrenia, Catatonic, Subchronic, Schizophrenia, Catatonic, Chronic, Schizophrenia, Catatonic, Subchronic with Acute Exacerbation, Schizophrenia, Catatonic, Chronic with Acute Exacerbation, Schizophrenia, Catatonic, in Remission, Schizophrenia, Catatonic, Unspecified, Schizophrenia, Disorganized, Chronic, Schizophrenia, Disorganized, Subchronic with Acute Exacerbation, Schizophrenia, Disorganized, Chronic with Acute Exacerbation, Schizophrenia, Disorganized, in Remission, Schizophrenia, Disorganized, Unspecified, Schizophrenia, Paranoid, Subchronic, Schizophrenia, Paranoid, Chronic, Schizophrenia, Paranoid, Subchronic with Acute Exacerbation, Schizophrenia, Paranoid, Chronic with Acute Exacerbation, Schizophrenia, Paranoid, in Remission, Schizophrenia, Paranoid, Unspecified, Schizophrenia, Undifferentiated, Subchronic, Schizophrenia, Undifferentiated, Chronic, Schizophrenia, Undifferentiated, Subchronic with Acute Exacerbation, Schizophrenia, Undifferentiated, Chronic with Acute Exacerbation (295.94), Schizophrenia, Undifferentiated, in Remission, Schizophrenia, Undifferentiated, Unspecified, Schizophrenia, Residual, Subchronic, Schizophrenia, Residual, Chronic, Schizophrenia, Residual, Subchronic with Acute Exacerbation, Schizophrenia, Residual, Chronic with Acute Exacerbation, Schizophrenia, Residual, in Remission, Schizophrenia, Residual, unspecified, Delusional (Paranoid) Disorder, Brief Reactive Psychosis, Schizophreniform Disorder, Schizoaffective Disorder, induced Psychotic Disorder, Psychotic Disorder NOS (Atypical Psychosis), Bipolar Disorder, Mixed, Severe, without Psychotic Features, Bipolar Disorder, Manic, Severe, without Psychotic Features, Bipolar Disorder, Depressed, Severe, without Psychotic Features, Bipolar Disorder, Mixed, with Psychotic Features, Bipolar Disorder, Manic, with Psychotic Features, Bipolar Disorder, Depressed, with Psychotic Features, Bipolar Disorder NOS, Major Depression, Single Episode, with Psychotic Features, Major Depression, Recurrent with Psychotic Features Personality Disorders, Paranoid Personality Disorders, Schizoid, Personality Disorders, Schizotypal, Personality Disorders, Antisocial, Personality Disorders and Borderline.

17. The method of claim 1 wherein the treatment is for Anxiety Disorders, Panic Disorder), Panic Disorder with Agoraphobia, Panic Disorder without Agoraphobia, Agoraphobia without History of Panic Disorders, Social Phobia, Simple Phobia, Organic Anxiety Disorder, Psychoactive Substance Anxiety Disorder, Separation Anxiety Disorder, Avoidant Disorder of Childhood or Adolescence, and Overanxious Disorder.

18. The method of claim 1 wherein the treatment is for cardiovascular disease includes strokes and any condition of the systemic vasculature and includes atherosclerosis, chronic heart failure and general heart disease.

19. A compound of general Formula (I) 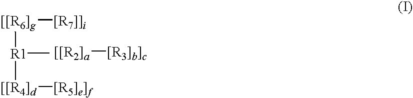
wherein
R₁ is a saturated or unsaturated hydrocarbon chain of from about 9 to about 26 carbon atoms and which is optionally carries one or more of a oxa, thia, hydroxy, hydroperoxy, epoxy and peroxy substitution;
each of R₂, R₄ and R₆ is selected from O₂, NO, NO₂, S(O)ₓ, C(H)_{y}, H, COOH, P(X)_{δ}(Y), N(H)_{z}, C═O, OH, 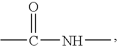
C₁₋₆ alkyl, C₁₋₆ alkoxy, amino, mono-acid di-C₁₋₆ alkylamino, C₁₋₆ alkylthio, S(O)ₓ—C₁₋₃ alkyl, C₁₋₆ alkoxycarbonyl, halo selected from fluoro, chloro, bromo and iodo, oxo, amidino and guanidino, C₂₋₁₂ alkenyl, C₂₋₁₂ alkynyl, aryl, heteroaryl and cyano, wherein x and z are 0, 1 or 2 and y is 0, 1, 2 or 3 and X is O, S or NR₈, Y is OR₉, SR₁₀ or NR₁₁R₁₂ and R₈, R₉, R₁₀, R₁₁, and R₁₂ are selected from H, alkyl, alkenyl, alkynyl, aryl and heteroaryl, δ is 0 or 1;
each of R₃, R₅ and R₇ is respectively [(CH₂)ⱼ(COOH)ₖ]ₗ, [(CH₂)ₘ(COOH)ₙ]ₒ and [(CH₂)ₚ(COOH)_{q}]ᵣ, wherein each of j, m and p is 0, 1, 2, 3, 4, 5 or 6, each of k, n and q is 0, 1 or 2, and each of l, o and r is 0 or 1,
each of c, i and f is 0 or 1 or 2; and
each of a, d and g is 0 or 1 or 2;
each of b, e and h is 0 or 1 or 2.
